(19) United States
(12) Patent Application Publication
(10) Pub. No.: US 2010/0204766 A1
(43) Pub. Date: Aug. 12, 2010

(54) IMPLANTABLE INTEGRATED CIRCUIT

(76) Inventors: Mark Zdeblick, Portola Valley, CA (US); Robert Leichner, Menlo Park, CA (US); Behrad Aria, Alameda, CA (US); Nilay Jani, Palo Alto, CA (US); Haifeng Li, Mountain View, CA (US); Todd Thompson, San Jose, CA (US); Lawrence Arne, Redwood City, CA (US); Timothy Robertson, Belmont, CA (US); Yafei Bi, Fremont, CA (US); Vladimier Gelfandbein, Mountain View, CA (US)

Correspondence Address: Proteus Biomedical, Inc.;Bozicevic, Field & Francis LLP, 1900 University Avenue, Suite 200, East Palo Alto, CA, 94303, US

(21) Appl. No.: 12/097,959

(22) PCT Filed: Dec. 22, 2006
(86) PCT No.: PCT/US06/48944

§ 371 Date: Jan. 22, 2010

Related U.S. Application Data
(60) Provisional application No. 60/753,863, filed on Dec. 22, 2005.
(60) Provisional application No. 60/753,598, filed on Dec. 22, 2005.
(60) Provisional application No. 60/763,478, filed on Jan. 30, 2006.
(60) Provisional application No. 60/773,699, filed on Feb. 14, 2006.
(60) Provisional application No. 60/745,272, filed on Apr. 20, 2006.
(60) Provisional application No. 60/805,060, filed on Jun. 16, 2006.
(60) Provisional application No. 60/820,065, filed on Jul. 21, 2006.
(60) Provisional application No. 60/820,588, filed on Jul. 27, 2006.
(60) Provisional application No. 60/829,828, filed on Oct. 17, 2006.
(60) Provisional application No. 60/868,041, filed on Nov. 30, 2006.

(51) Int. Cl. A61N 1/05

(52) U.S. Cl. 607/119

(57) ABSTRACT

Embodiments of the present invention enable robust, reliable control functionality for effectors present on intraluminal, e.g., vascular leads, as well as other types of implantable devices. Embodiments of the invention enable the required functionality for accurate long term control of effectors units, even ones present on multiplex carrier configurations, while provide for low power consumption. Aspects of the invention include implantable integrated circuits that have power extraction; energy storage; communication; and device configuration functional blocks, where these functional blocks are all present in a single integrated circuit on an intraluminal-sized support. Also provided by the invention are effector assemblies that include the integrated circuits, as well as implantable medical devices, e.g., pulse generators that include the same, as well as systems and kits thereof and methods of using the same, e.g., in pacing applications, including cardiac resynchronization therapy (CRT) applications.

### CROSS-REFERENCE TO RELATED APPLICATIONS

Pursuant to 35 U.S.C. §119 (e), this application claims priority to the filing date of: U.S. Provisional Patent Application Ser. No. 60/753,863 filed Dec. 22, 2005; U.S. Provisional Patent Application Ser. No. 60/753,598 filed Dec. 22, 2005; U.S. Provisional Patent Application Ser. No. 60/763,478 filed Jan. 30, 2006; U.S. Provisional Patent Application Ser. No. 60/773,699 filed Feb. 14, 2006; U.S. Provisional Patent Application Ser. No. 60/745,272 filed Apr. 20, 2006; U.S. Provisional Patent Application Ser. No. 60/805,060 filed Jun. 16, 2006; U.S. Provisional Patent Application Ser. No. 60/820,065 filed Jul. 21, 2006; U.S. Provisional Patent Application Ser. No. 60/820,588 filed Jul. 27, 2006; U.S. Provisional Patent Application Ser. No. 60/829,828 filed Oct. 17, 2006; and U.S. Provisional Patent Application Ser. No. 60/868,041 filed Nov. 30, 2006; the disclosures of which applications are herein incorporated by reference.

This application is also a continuation-in-part application of application Ser. No. 11/219,305 filed on Sep. 1, 2005, which application claims priority under 35 U.S.C. §119 from the following provisional applications: U.S. Provisional Patent Application No. 60/707,995, filed Aug. 12, 2005; U.S. Provisional Patent Application No. 60/679,625, filed May 9, 2005; U.S. Provisional Patent Application No. 60/638,928, filed Dec. 23, 2004; and U.S. Provisional Patent Application No. 60/607,280, filed Sep. 2, 2004; the disclosures of which applications are herein incorporated by reference.

### INTRODUCTION

### Background

The history of biomedical implantable devices traces back to its beginning in the late 1950s. Since the first development of the implantable cardiac pacemaker over forty years ago, the field of bioengineering has provided many different implantable biomedical devices to the medical profession for the treatment of various conditions. Today, implantable cardioverter/defibrillators, drug delivery systems, neurological stimulators, bone growth stimulators, and many other implantable devices significantly facilitate the treatment of a variety of diseases.

For any type of implantable devices, to be able to precisely control the behavior and to accurately monitor the state of these devices is critically important for effective treatment of the illness. For instance, in cardiac resynchronization therapy (CRT), a pacing lead is often inserted into a patient's heart. The location and timing of the pacing signals applied to the heart tissue can drastically affect the effectiveness of the resynchronization therapy. Ideally, a physician can use an implantable device to monitor the response of the tissue and the state of the implantable device to evaluate the efficacy of the treatment.

The development of biomedical implantable devices reflects, in many ways, the development of electronic technology, particularly the progress in the areas of microelectronics, circuit design, sensing technology, micro electro-mechanical systems (MEMS), signal processing, and other related fields. However, the latest electronic technologies are often not incorporated in the implantable devices, due to a lack of large-scale collaborative efforts among electrical engineering, bioengineering, and medical science. For example, until recently, a large number of cardiac resynchronization therapists still relied on semi-empirical methods to adjust the pacing lead and pacing signals.

At present, there are only limited applications of automatically controlled implantable devices such as pace makers and neurological stimulators. Moreover, automatic operation of the existing implantable devices often requires bulky external control systems and power sources. Such operation can be difficult to administer and often impossible to manage outside the clinic.

### SUMMARY

Embodiments of the present invention enable robust, reliable control functionality for effectors present on intraluminal, e.g., vascular leads, as well as other types of implantable devices. Embodiments of the invention enable the required functionality for accurate long term control of effectors units, even ones present on multiplex carrier configurations, while providing for low power consumption. Aspects of the invention include implantable integrated circuits that have power extraction; energy storage; communication; and device configuration functional blocks, where these functional blocks are all present in a single integrated circuit on an intraluminal-sized support. Also provided by the invention are effector assemblies that include the integrated circuits, as well as implantable medical devices, e.g., pulse generators that include the same, as well as systems and kits thereof and methods of using the same, e.g., in pacing applications, including cardiac resynchronization therapy (CRT) applications.

### BRIEF DESCRIPTION OF THE FIGURES

FIG. 1 illustrates the locations of a number of pacing satellites incorporated in multi-electrode pacing leads, in accordance with an embodiment of the present invention.

FIG. 2 illustrates an exemplary external view of a number of pacing satellites, in accordance with an embodiment of the present invention.

FIG. 3 is a high-level block diagram for a control circuitry within a satellite on a multi-satellite lead, in accordance with an embodiment of the present invention.

FIG. 4 illustrates an implantable pacemaker lead that is operable to function in a default mode, according to an embodiment of the present invention.

FIG. 5 illustrates an implantable pacemaker lead that is operable to function in a unipolar default mode before receiving a power supply voltage, according to another embodiment of the present invention.

FIG. 6 illustrates an implantable pacemaker lead that is operable to function in a bipolar default mode before receiving a power supply voltage, according to yet another embodiment of the present invention.

FIG. 7A illustrates the input portion of one-shot circuitry that generates a one-shot pulse to initiate a default mode of operation in an implantable pacemaker lead, according to an embodiment of the present invention.

FIG. 7B continues one-shot circuitry that generates a one-shot pulse to initiate a default mode of operation in an implantable pacemaker lead, according to an embodiment of the present invention.

FIGS. 8A-E illustrates a register array circuit that places an implantable pacemaker lead in a default mode of operation, according to an embodiment of the present invention.

FIG. 9 is a high-level block diagram illustrating a simple power-supply circuit which could cause the electrode-switching circuit as is illustrated in FIG. 6 to malfunction during charge-balanced pacing.

FIG. 10 is an exemplary voltage waveform for a charge-balanced pacing cycle.

FIG. 11 presents an exemplary electrode-switching circuit that could malfunction during charge-balanced pacing.

FIG. 12 is a schematic circuit diagram for a power supply circuit that provides three voltages for a portion of the control circuitry, in accordance with an embodiment of the present invention.

FIG. 13 is a schematic circuit diagram illustrating an electrode-switching circuit that can withstand large voltage swings and polarity changes during charge-balanced pacing, in accordance to one embodiment of the present invention.

FIG. 14 is a schematic circuit diagram illustrating a power-supply circuit that provides two switch-control signals, vhigh_logic_S1 and vhigh_logic_S2, to the electrode-switching circuit as is illustrated in FIG. 13, in accordance with an embodiment of the present invention.

FIG. 15 is a schematic circuit diagram illustrating a power-supply circuit that provides two switch-control signals, vlow_logic_s1 and vlow_logic_s2, to the electrode-switching circuit as is illustrated in FIG. 13, in accordance to an embodiment of the present invention.

FIG. 16 is a diagram of a pacemaker can that is coupled to an implantable pacemaker lead.

FIG. 17 illustrates a cylindrical blocking capacitor in a pacemaker lead that includes an electrode, a dielectric layer, and a second conductive layer formed on the dielectric, according to one embodiment of the present invention.

FIG. 18 illustrates a cylindrical blocking capacitor in a pacemaker lead that includes an electrode, a dielectric layer, and patient tissue that acts as a second conductive layer, according to another embodiment of the present invention.

FIG. 19A illustrates four blocking capacitors formed in a cylindrical shape in a pacemaker lead, according to another embodiment of the present invention.

FIG. 19B illustrates four blocking capacitors formed in a cylindrical shape in a pacemaker lead that each include an electrode, a dielectric layer, and a second conductive layer formed on the dielectric, according to another embodiment of the present invention.

FIG. 19C illustrates four blocking capacitors formed in a cylindrical shape in a pacemaker lead that each include an electrode, a dielectric layer, and patient tissue that acts as the second conductive layer, according to another embodiment of the present invention.

FIGS. 20A-20C illustrate blocking capacitors in pacemaker leads that have irregular surfaces, according to further embodiments of the present invention.

FIG. 21 illustrates a blocking capacitor formed on the surface of a helical screw-in electrode, according to an embodiment of the present invention.

FIG. 22 illustrates blocking capacitors in a pacemaker lead that are coupled in between electrodes and a multiplexer, according to an embodiment of the present invention.

FIG. 23 is schematic representation of the switching circuit of the present invention operatively interfaced between a pacemaker and a plurality of electrical leads for implantation within the heart;

FIG. 24 is a schematic representation of one embodiment of a switching circuit of the present invention;

FIG. 25 is a schematic representation of another embodiment of a switching circuit of the present invention; and

FIG. 26 is a schematic representation of another embodiment of a switching circuit of the present invention.

FIG. 27 illustrates a variation of the controller interface.

FIG. 28 illustrates an example of a pacemaker connected to an implantable medical device having multiple electrodes that can provide fault recovery, according to an embodiment of the present invention.

FIG. 29A illustrates an example of an implantable device having two leads that are coupled to multiple satellite devices, which are coupled to multiple electrodes.

FIG. 29B illustrates an example of an implantable device having a single lead that is coupled to multiple satellite devices, which are coupled to multiple electrodes.

FIG. 30 illustrates an implantable device having multiple faults.

FIG. 31 illustrates a system including an implantable device that can recover from a failure on a lead in the implantable device by sending signals through an auxiliary lead, according to an embodiment of the present invention.

FIG. 32 illustrates an implantable device that can isolate an element within a satellite containing a failure to provide fault recovery, according to another embodiment of the present invention.

FIG. 33 illustrates an implantable device that can break electrical connections between two ends of an element in the device to provide fault recovery, according to another embodiment of the present invention.

FIG. 34 illustrates an implantable device having leads that are coupled to a satellite and a logic element for providing fault recovery, according to yet another embodiment of the present invention.

FIG. 35 illustrates a technique for creating an electrical open circuit in a conductor without compromising the strength of the non-conductive core, according to another embodiment of the present invention.

FIG. 36 illustrates a technique for creating an electrical open circuit in a sheath conductor without compromising the strength of the non-conductive core, according to a further embodiment of the present invention.

FIG. 37 illustrates how a pacemaker can is able to detect a fault in a satellite device coupled to leads in an implantable device by sequentially powering the satellites, according to an embodiment of the present invention.

FIG. 38 illustrates another system for recovery from a fault condition in an implantable medical device, according to a further embodiment of the present invention.

FIG. 39 is a schematic view of one embodiment of the inventive overvoltage protection configuration.

FIG. 40 is a schematic view of an embodiment providing a sensing capacity.

FIGS. 41A-C illustrate a more complex embodiment of the inventive circuitry.

FIG. 42 shows an expanded view of the deliberation output module of the circuitry shown in FIG. 41.

FIGS. 43-47 provide a diagrammatic view of the general concepts of the present inventive circuitry.

FIG. 48 is a block diagram illustrating a configuration that uses transistor-based current limiting circuitry to protect the satellites and tissue from over current, in accordance with one embodiment.

FIG. 49 is a schematic circuit diagram illustrating a uni-directional current limiting circuitry, in accordance with one embodiment.

FIG. 50 is a schematic circuit diagram illustrating a bi-directional current limiting circuitry, in accordance with one embodiment.

FIG. 51 illustrates an exemplary scenario where a defibrillation electrical field results in a voltage drop between two pacing satellites.

FIG. 52 illustrates an exemplary scenario where two pacing satellites without overcurrent protection allow a high-density current to pass through the tissue surrounding the electrodes during a defibrillation process.

FIG. 53 illustrates an exemplary configuration of two pacing satellites where diodes are used to prevent the formation of a low-impedance circuit.

FIG. 54 presents a schematic circuit diagram illustrating a configuration that uses transistors to isolate an electrode from a bus wire in accordance with an embodiment of the present invention.

FIG. 55 presents a schematic circuit diagram illustrating a configuration that uses current mirrors to isolate an electrode from a bus wire in accordance with an embodiment of the present invention.

FIGS. 56A-H provide a flow diagram of a fabrication method for the inventive integrated off-chip capacitor design.

FIG. 57 illustrates an IC device attached to the inventive integrated off-chip capacitor.

FIG. 58 shows an embodiment of the implantable on-chip capacitor in which two electrodes are deposited on a substrate

FIG. 59 shows an embodiment of the implantable on-chip capacitor in which two electrodes are deposited as columns on the substrate.

FIG. 60 shows an embodiment of the implantable on-chip capacitor in which two electrodes are deposited on opposite sides of the substrate.

FIG. 61 shows the top view of an embodiment of the implantable on-chip capacitor in which an electrode column is surrounded and separated by another electrode ring.

FIG. 62 is a data curve representing the capacitance of platinum iridium.

FIG. 63 is a data curve representing the open circuit voltage of a platinum iridium capacitor.

FIG. 64 shows the embodiment of an effector covered by a highly porous material.

FIG. 65 shows an embodiment of the implantable on-chip capacitor in which multiple capacitors are connected in series.

FIG. 66 shows another embodiment of the implantable on-chip capacitor in which two capacitors are connected in series.

FIG. 67 shows another embodiment of the implantable on-chip capacitor in which five capacitors are connected in series.

FIG. 68 shows an embodiment of the present invention, in which multiple control circuits are connected in parallel along two bus wires.

FIG. 69 shows an embodiment of the data encoding scheme used for communication.

FIG. 70 shows an embodiment of the power generation block.

FIG. 71 shows an embodiment of the data clock recovery block.

FIG. 72 shows an embodiment of the inventive wakeup circuitry.

FIG. 73 shows another embodiment of the inventive wakeup circuitry.

### DETAILED DESCRIPTION

As summarized above, embodiments of the present invention enable robust, reliable control functionality for effectors present on intraluminal structures, e.g., vascular leads, as well as other types of implantable devices. Embodiments of the invention enable the required functionality for accurate long term control of effectors units of the implantable structure, even ones present on multiplex carrier configurations, while providing for low power consumption. Such advantages provided by embodiments of the present invention enable a variety of different enhanced implantable technologies, such as enhanced implantable pulse generators, e.g., cardiac pacing devices.

Aspects of the invention include implantable integrated circuits that have power extraction, energy storage, communication, and device configuration functional blocks, where these functional blocks are all present in a single integrated circuit on an intraluminal-sized support. Also provided by the invention are effector assemblies that include the integrated circuits, as well as implantable medical devices, e.g., pulse generators that include the same, as well as systems and kits thereof and methods of using the same, e.g., in pacing applications, including cardiac resynchronization therapy (CRT) applications.

In further describing various aspects of the invention, embodiments of the inventive integrated circuits will be reviewed first in greater detail, both generally and in terms of the figures, followed by a discussion of implantable medical devices that may include the subject circuits and systems thereof, as well as a review of various kits thereof.

### Integrated Circuits

Embodiments of the invention provide implantable integrated circuits. By implantable is meant that the circuits are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable circuits are configured to maintain functionality when implanted at a physiological site for a period ranging from about 1 to about 80 years or longer, such as from about 5 to about 70 years or longer, and including for a period ranging from about 10 to about 50 years or longer.

The implantable integrated circuits include a number of distinct functional blocks, i.e., modules, where the functional blocks are all present in a single integrated circuit on an intraluminal-sized support. By single integrated circuit is meant a single circuit structure that includes all of the different functional blocks. As such, the integrated circuit is a monolithic integrated circuit (also known as IC, microcircuit, microchip, silicon chip, computer chip or chip) that is a miniaturized electronic circuit (which may include semiconductor devices, as well as passive components) that has been manufactured in the surface of a thin substrate of semiconductor material. The integrated circuits of certain embodiments of the present invention are distinct from hybrid integrated circuits, which are miniaturized electronic circuits constructed of individual semiconductor devices, as well as passive components, bonded to a substrate or circuit board.

The support with which the circuit is associated, e.g., by being present on surface of the support or integrated, at least partially, inside of the support, may be any convenient support, and may be rigid or flexible as desired. As the support is intraluminal sized, its dimensions are such that it can be positioned inside of a physiological lumen, e.g., inside of a vessel, such as a cardiac vessel, e.g., a vein or artery. In certain embodiments, the intraluminal sized integrated circuits have a size (e.g., in terms of surface area of largest surface) of between about 0.05 mm² and about 5 mm², such as between about 1.125 mm² and about 2.5 mm², and including about 1.5 mm². The supports of the integrated circuits can have a variety of different shapes, such as square, rectangle, oval, and hexagon, irregular, etc.

As indicated above, the integrated circuits of the invention may include a number of functional blocks which provide for the requisite functionality of the circuit for its intended use, where the functional blocks are all part of a single integrated circuit. In certain embodiments, the circuits include at least the following functional blocks: a power extraction functional block; an energy storage functional block; a communication functional block; and a device configuration functional block.

The power extraction functional block is a circuitry functional block or module that is configured to extract or obtain power from a power source to which the circuit is coupled. In the broadest sense, the power extraction functional block may be a block that is configured to receive power from an electrically coupled source, e.g., wire, or remotely, e.g., power that is wirelessly transmitted to the circuit from a remote location, where that remote location may be an in vivo or ex vivo location, but is one that is not physically connected to the device by a conductive element, such as a wire. In certain embodiments, the power extraction functional block is one that is configured to be coupled to at least one wire that is, in turn, coupled to a power source, such as a battery, where the functional block extracts power from the wire to power the circuit.

Another functional block or module present that is part of the integrated circuit is an energy storage functional block. The energy storage functional block is one that is capable of storing energy in the circuit, e.g., in a capacitor fashion, such as the energy extracted by the power extraction block. The energy storage functional block has, in certain embodiments, an energy storage capacity of about 200 pF or more, such as about 500 pF or more, including about 800 pF or more, and in certain embodiments the storage capacity of the block is about 5000 pF or less, such as about 2000 pF or less, including about 1000 pF or less. As such, the storage capacity of the functional block may, in certain embodiments have a total capacity ranging from about 200 to about 5000 pF, such as from about 500 to about 2500 pF, including from about 750 to about 2000 pF. This functional block may be made up of a single discreet circuit element or multiple circuit elements, e.g., two or more, three or more, etc., elements each having a capacity ranging from about 60 to about 220 pF, etc.

The circuits of these embodiments further include a communication functional block. This block provides for sending and receiving of data, e.g., in the form of signals, from a location remote to the integrated circuit, be that location in vivo or ex vivo, where the location may be physically connected to the circuit or not. In certain embodiments, this block is configured to receive command signals from a control unit that is connected to the circuit via at least one wire and/or transmit sensed data signals from the circuit to a control unit over at least one wire, where the control unit is remote from the circuit and physically connected to the circuit by the at least one wire. In certain embodiments, the communication functional block employs an alternating current at a frequency above about 15 kHz, where the operating frequency of the communication functional block may be about 100 kHz or more, such as about 500 kHz or more, including about 1 MHz or more.

The circuit further includes a device configuration functional block. This block is able to employ configuration commands, e.g., as received from a remote device via the communication block, and configure one or more effectors of the device, e.g., electrodes, according to the received configuration command. In certain embodiments, the device configuration functional block is configured such that the device configuration provided by the functional block of the integrated circuit is functional without power being applied to said integrated circuit. In certain embodiments, the device configuration block includes a switching block between supply terminals and one or more effectors. The switching block may include switching elements each made up of two transistors between each effector and supply terminal.

In certain embodiments, the two transistors share a common bulk that is electrically isolated from all other circuits. In other embodiments, the two transistors include gates that are electrically connected. The two transistors can include sources that are connected. The common bulk can be electrically connected to a common source terminal. The circuit may also be configured such that during use a control voltage applied to the gates is referenced to a voltage on the supply terminal.

Various examples of the above functional blocks are further described below, both generally and in terms of the figures, where the above components may be described in the context of circuits that include additional functional blocks.

In certain embodiments, in a given device or system, such as the devices and systems described below, substantially all, if not all of the functions of power extraction, energy storage, communication and device configuration employed by the integrated circuit during use are provided by the single integrated circuit. In yet other embodiments, the device or system in which the circuit is present may provide some of the above functionalities. However, even in such embodiments, the circuits may still include the above summarized functional blocks.

In certain embodiments, the integrated circuits are configured to be employed in therapeutic cardiac applications, such as cardiac function monitoring applications and/or therapeutic electrical energy delivery applications, e.g., pacing applications. As such, the circuits may include a functional block that enables stimulation of tissue via an effector, e.g., electrode, that is coupled to the circuit. The circuits may include a functional block that enables low voltage transmission from tissue, e.g., that is contacting an effector coupled to the circuit, to the integrated circuit. In certain embodiments, the integrated circuit may provide a substantially charge-balanced transmission of a stimulation pulse to tissue, e.g., that is contacting an effector which is coupled to the circuit.

The integrated circuits may include a number of additional functionalities imparted to the circuit by one or more additional functional blocks. Some of these functionalities are summarized below and then further developed, e.g., in connection with description of the figures of the application. All or just some of the components required for the following functionalities may be integrated into the circuit. As such, a given functional block as described above, is a functional block that, by itself or in conjunction with additional elements not integrated in the circuit, provides for the desired additional functionality. The functional blocks include a default mode functional block, a charge balanced operation functional block, a charge-balanced functional block, a multiplexer functional block, a fault tolerant functional block, an overvoltage and/or overcurrent functional block, an off-chip or on chip capacitor functional block, sleep functional block, and a wakeup functional block. In various embodiments, these additional functionalities further enable the integrated circuits of the invention to have their intraluminal size and low power consumption and yet provide for desired functionality.

Each of the above specified additional functional blocks is summarized below and then reviewed in greater detail in the specification, including in connection with figures of various embodiments.

### Default-Mode Operation

In one embodiment, the integrated circuits are configured to be operable in a default mode, e.g., where the circuits are employed in electrode assemblies on a lead, such as a multi-electrode lead (MEL). In such an embodiment, the circuits include a default mode functional block, which enables the circuit and assembly coupled thereto to operate in a default mode without the electrodes being first powered up and configured. As such, in these embodiments a device configuration provided by said integrated circuit is functional without power being applied to said integrated circuit.

This default-mode operation allows an implantable medical device, such as a MEL, to operate without consuming extra power for electrode configuration. Furthermore, the default-mode operation allows the MEL to easily interoperate with conventional pacing systems. In such embodiments, the integrated circuit may have a functional block that enables default operation, e.g., as described above.

In certain embodiments, the circuit is configured to have a default configuration connecting one supply terminal to one or more effectors upon power up of said circuit. As such, in these embodiments, upon power up of the circuit, the circuit assumes a default configuration with respect to one or more effectors that are coupled to the circuit, without receiving any configuration data from a remote source.

### Charge Balanced Operation Functional Block

In a further aspect of the present invention, the control device on each satellite facilitates charge-balanced operation, thereby significantly extending the lifetime of electrodes. As such, embodiments of the invention include functional blocks that enable an integrated circuit to provide substantially charge-balanced transmission of a stimulation pulse.

### Multiplexer Functional Block

Furthermore, embodiments of the present invention provide a multiplexing system that allows signals detected by different satellites to be multiplexed and transmitted to a separate data collection system using the same two bus wires which are used to drive the electrodes. Embodiments include modular circuits which are physically implantable adjacent to and electrically coupled between a pacemaker and the associated electrical leads.

The modular circuits provide a communication link between the pacemaker and the plurality of electrodes and/or a plurality of sensors which are associated with the leads, and more particularly, communicates the input and output signals between the pacemaker and the electrodes and their associated electrode circuitry. More particularly, the multiplexing provides latches that can be controlled to connect to or disconnect from the pacemaker any of the electrodes associated with a given pacing lead.

The subject circuits are able to maintain the various electrodes in their respective assigned state i.e., active or inactive, while minimizing leakage currents. In addition to controlling electrodes and sensors implanted within the body, the subject circuits also function as a communication link to devices external to the patient's body.

### Fault Tolerant Operation Functional Block

In certain embodiments, the integrated circuit further includes a fault recovery functional block, where the fault recovery functional block is configured to electrically isolate failed circuits or wires in a system in which the circuit is present. The present invention also provides fault-recovery mechanisms to protect implantable medical device or system from selected failures, e.g., protect a MEL system in the event where one or more satellites or part of a bus wire fails.

### Overvoltage and Overcurrent Protection Functional Blocks

The present invention provides strategies for circuitry configurations that provide both overcurrent protection in the circuit to avoid inadvertent tissue damage and overvoltage protection of circuitry. In certain embodiments, the integrated circuit further includes a current limiting functional block. In certain embodiments, the integrated circuit further includes a voltage-clamping functional block. Further embodiments of the present invention provide over-voltage and over-current protections. These protection circuitry configurations ensure undisrupted operation and protection of the MEL during defibrillation processes wherein the patient's tissue is subject to a high-voltage or high-current electrical pulse.

### Off-Chip Capacitor and On-Chip Capacitor Functional Blocks

In addition, the present invention provides novel on-chip and off-chip capacitor designs which allow the control device to reduce its chip size, increase the MEL's flexibility, and facilitate a wide range of applications of the MEL. Integrated circuits of embodiments of the invention include functional blocks for enabling such components.

### Sleep/Wakeup Functional Blocks

In certain embodiments, the integrated circuits further include a sleep functional block. This sleep functional block is responsive to a sleep signal, e.g., as may be transmitted from a remote control unit, and upon receipt of such a signal, shuts down certain portions of the circuit, e.g., so that the “sleeping” portions do not consume power. In certain embodiments, the integrated circuits further include a wakeup functional block, which is responsive to a wakeup signal, e.g., is activated by the wake up signal, such as an encoded wakeup signal, and upon receipt of such a signal turns on portions of the circuit that are shut down.

Where desired, the integrated circuit may include one or more integrated corrosion protection films, e.g., which serve as primary protection of the circuit and functional blocks thereof from the implanted environment and impart the implantable functionality to the circuit, e.g., as described above. In certain of these embodiments, the integrated corrosion protection films are planar deposited corrosion protection films. In certain embodiments, the protection films, i.e., layers, are those described in U.S. Provisional Application Ser. No. 60/791,244 titled “Void-Free Implantable Hermetically Sealed Structures” and filed Apr. 12, 2006; the disclosure of which is herein incorporated by reference.

These aforementioned features individually or jointly contribute to embodiments of the realization of a low power-consumption, intraluminally sized control devices which provide desired functionality in implantable medical devices.

In certain embodiments, the integrated circuit is characterized by having low power consumption while providing necessary functions for automated actuating or sensing, e.g., from multiple electrodes or sensors which may be coupled to the integrated circuit. Particularly, the modular components of the underlying integrated circuit and related circuitry consume significantly reduced amounts of power, e.g., as compared to non-integrated circuits that may include similar functionalities, thereby allowing the entire implantable pacing/sensing system with which the integrated circuit is associated to operate with limited power source, such as may be provided by a battery included in a pacing can.

According to one embodiment, the average power consumption of each integrated circuit is about 100 μW or less, such as about 100 nW or less, and including about 50 pW or less. The average current draw of the inventive integrated circuit while maintaining its configuration state is about 1 nA or less, including about 5 pA or less. In addition, the average current draw of the inventive integrated circuit when the configuration state of the device is being changed ranges from about 1 μA to about 100 μA, such as from about 10 μA to about 50 μA, and including from about 1 μA to about 20 μA.

In one embodiment, the integrated circuit is associated with a number of electrodes, e.g., that may be present in a satellite structure of a lead, where multiple satellites may reside on a single implantable lead. The inventive implantable integrated circuits facilitate selecting and driving electrodes on such satellites and/or sensing signals through these electrodes. Furthermore, the inventive integrated circuits facilitate relaying data back from an electrode to a data collection system, so that the signals detected by the electrodes can be processed and analyzed. In such embodiments, the inventive integrated circuits may also allow a satellite to maintain its configuration state once the satellite and its electrodes are configured. The satellites can retain their respective configuration states while the external power supply is turned off. Hence, the power consumption for the entire implantable signal administration/detection system can be significantly reduced compared with conventional systems.

Another aspect of embodiments of the present invention is that the electrodes and satellites are given a default configuration state. That is, the electrodes in the implantable satellites are by default uncoupled or coupled to one of the bus wires within the lead, even when no power is provided through the lead. This aspect allows the present inventive implantable satellites and electrodes to inter-operate with existing pacing systems which cannot provide complex digital programming commands. Moreover, this aspect also allows the implantable electrodes to be readily operational without any prior power-up or configuration.

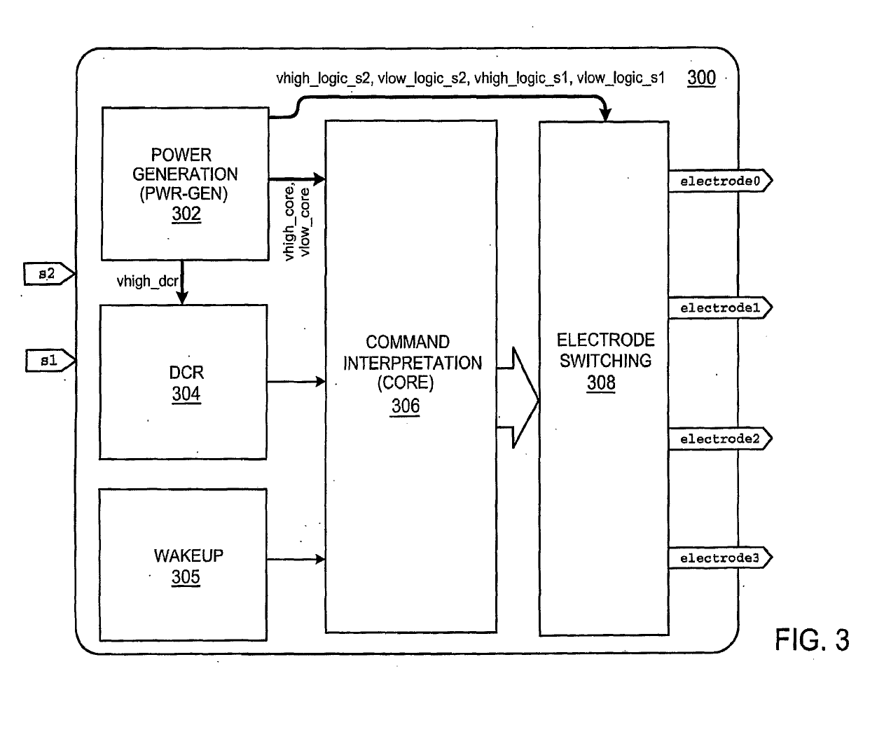

FIG. 3 is a high-level block diagram for an integrated circuit of an embodiment of the invention that includes control circuitry for a satellite structure that may be present on a multi-satellite lead, in accordance with an embodiment of the present invention. Control circuit 300 includes a power generation (PWR-GEN) module 302 (which is a power extraction block), a data-clock recovery (DCR) module 304, a wakeup module 305, a command interpretation module 306 (referred to as the “CORE” module in one embodiment), and an electrode-switching module 308 which is coupled to four electrodes.

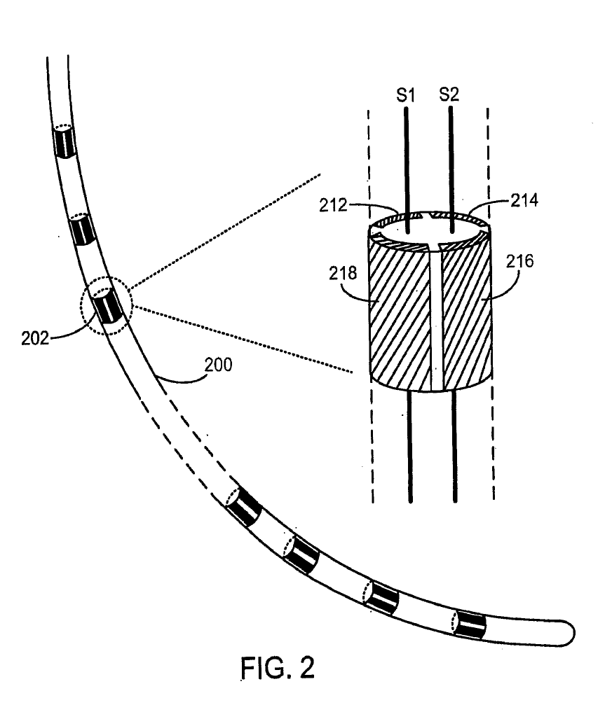

DCR module 304 provides the correct clock signals recovered from signals, as may be carried on bus wires S1 and S2 (See e.g., FIG. 2 described in greater detail below) to the rest of digital circuitry within control chip 300. DCR module 304 also recovers the data signals carried on S1 and S2 into a digital format that can be used by CORE module 306.

Wakeup module 305 generates a wakeup signal to activate and initialize other modules after a dormant period during which circuits within control chip 300 are turned off to preserve power.

CORE module 306 generates the proper control signals, based on the data received from DCR module 304, to control electrode-switching module 308. Electrode-switching module 308 then selects and switches the electrodes so that the desired electrodes can couple to S1 or S2 for pacing and/or signal-detection purposes.

PWR-GEN module 302 generates the power-supply voltages for CORE module 306, DCR module 304, and electrode-switching module 308. Specifically, PWR-GEN module 302 provides two voltages, vhigh_core and vlow_core, to CORE module 306, and a high voltage, vhigh_dcr, to DCR module 304. Furthermore, PWR-GEN module 302 provides four switch-control signals, vhigh_logic_S2, vlow_logic_s2, vhigh_logic_S1, and vlow_logic_s1, to electrode-switching module 308. These four switch-control signals ensure the electrode-switching circuits to turn on or off sufficiently under large S2−S1 voltage swings incurred during charge-balanced pacing.

### Additional Functionalities

As summarized above, the integrated circuits of the invention may include or be coupled to additional components that provide for a number of different desired functional abilities. Additional functionalities of interest include: Default-Mode Operation, Charge Balanced Operation, e.g., by using Blocking Capacitors for Charge-Balanced Operation; Fault Tolerant Operation; Overvoltage and Overcurrent Protection; Off-Chip and On-Chip Capacitance, DCR and Wakeup Operation. Each of these different functionalities of interest is now described in greater detail, both generally and in terms of figures.

Although the following description frequently uses cardiac pacing as an exemplary application, embodiments of the present invention can be applied by a wide range of applications wherein signals are administered to or detected from living tissues. Such applications include, but are not limited to: cardiac pacing and monitoring, neurological stimulation, bone growth stimulation, and drug delivery.

It should be noted that integrated circuits of the invention may have one or more functional blocks that enable the following functionalities. However, the following functionalities are not limited to their implementation in the integrated circuits of the device, but could appear in other implantable medical devices and systems that may not include the integrated circuits as summarized above. These additional medical devices and systems to the extent they include one or more of the following functionalities are specifically within the scope of this invention. As such, included within the scope of the invention are multielectrode leads which include one or more of the following functionalities, whether or not the functionalities are provided by an integrated circuit or some other device: Default-Mode Operation, Charge Balanced Operation, e.g., by using Blocking Capacitors for Charge-Balanced Operation; Fault Tolerant Operation; Overvoltage and Overcurrent Protection; Off-Chip and On-Chip Capacitance, DCR and Wakeup Operation.

### Default-Mode Operation

Embodiments of the present invention provide implantable devices, such as satellite units of a multi-electrode lead (MEL) that are operable in a default mode. Such devices include an integrated circuit which is configured such that it is operational upon power up whether or not it receives configuration data following power up. For example, a pacemaker lead of the present invention can operate in a default mode after it is coupled to a pacemaker can, regardless of whether it receives electrode configuration signals from the pacemaker can.

In the default mode, a pacemaker lead can provide pacing functions in response to pacing signals that fall within an accepted range. The ranges of signals that are accepted by a pacemaker lead are broad enough to include pacing signals generated by many different models of pacemaker cans. As such, pacemaker leads of the present invention are not limited to being used with only one pacemaker can model or one class of pacemaker cans made by a particular manufacturer. A pacemaker lead of the present invention can be used with almost any pacemaker can.

The present invention provides the ability to replace the pre-existing can with one from a wide variety of makes and models, should the need arise. This can be accomplished while using the existing pacemaker leads. This is desirable over performing an additional surgical procedure to replace the pre-existing pacemaker leads. It would be desirable if an implanted pacemaker lead could respond to pacing signals generated by one pacemaker model or a class of pacemaker models made by any manufacturer. This advantage is available through the present invention.

Pacemaker leads can include one or more integrated circuit chips. Each of the chips can include a set of switches (e.g., 4 switches). Each of the switches couple or decouple an anode wire or a cathode wire in the lead to an electrode. The switches are typically implemented by a set of transistors according to any convenient circuit design techniques.

A pacemaker lead of the present invention is connected to a pacemaker can. The pacemaker lead is operable in a default mode. In the default mode, the switches in the integrated circuit chips remain in or switch to a default configuration. When the switches are in the default configuration, one or more of the electrodes are coupled to the anode wire and/or the cathode wire.

In one approach, the switches in one or more chips can be switched to couple a corresponding electrode to an anode wire or a cathode wire. The switches can also decouple a corresponding electrode from both the anode wire and the cathode wire so that the pacemaker cannot send current to that electrode. Thus, each of the switches can be placed in one of three states: decoupled, coupled to the anode wire, or coupled to the cathode wire.

Some types of pacemaker cans are able to generate configuration signals that can control the states of the switches in the integrated circuit chips that are in an implantable pacemaker lead. These types of pacemaker cans are able to change the states of the switches in order to stimulate any of the electrodes in the lead in any desired pacing configuration.

However, other types of pacemaker cans cannot generate configuration signals for controlling the states of the switches. According to the present invention, one or more of the electrodes are coupled to the anode and/or cathode wire in a default mode. Therefore, a pacemaker can that is not able to generate configuration signals for changing the states of the switches is still able to send current to at least one of the electrodes in a default mode. The default configuration of the switches allows any pacemaker can that is able to generate pacing signals within an accepted range to stimulate the cardiac tissue and provide at least a basic pacing function.

According to some embodiments of the present invention, an implantable pacemaker lead is already in a functional default mode before the lead is coupled to a pacemaker can. According to other embodiments of the present invention, an implantable pacemaker lead enters a functional default mode after the lead is coupled to a pacemaker can, and the power supply voltage reaches or exceeds a predefined threshold voltage.

The integrated circuit chips on a pacemaker lead can be classified as three types of default mode chips: anode default, cathode default, and off default. Anode default chips contain switches that couple one or more electrodes to the anode wire in default mode. The DC lead impedance for an anode default chip can be, for example, in the range of about 20 to about 225Ω, such as from about 112 to about 225Ω, such as about 120Ω.

Cathode default chips contain switches that couple one or more electrodes to the cathode wire in default mode. The DC lead impedance for a cathode default chip can be, for example, in the range of about 15 to about 80Ω, such as from about 20 to about 80Ω, including about 40Ω. If the pulse amplitude of the pacing signals are increased, the lead impedances are reduced. Chips that are off by default contain switches that disconnect all of their electrodes from the anode and cathode wires. Chips that are off by default can be, for example, in the range of about mega ohm impedance until turned on using a pacemaker can.

A pacemaker lead of the present invention can have integrated circuit chips with any number of switches that are coupled to a corresponding number of electrodes. For example, in one instance, electrode configuration can be set to provide the patient an effective therapeutic procedure. In another instance, the electrode configuration can be reset to provide the same patient a more effective therapeutic procedure.

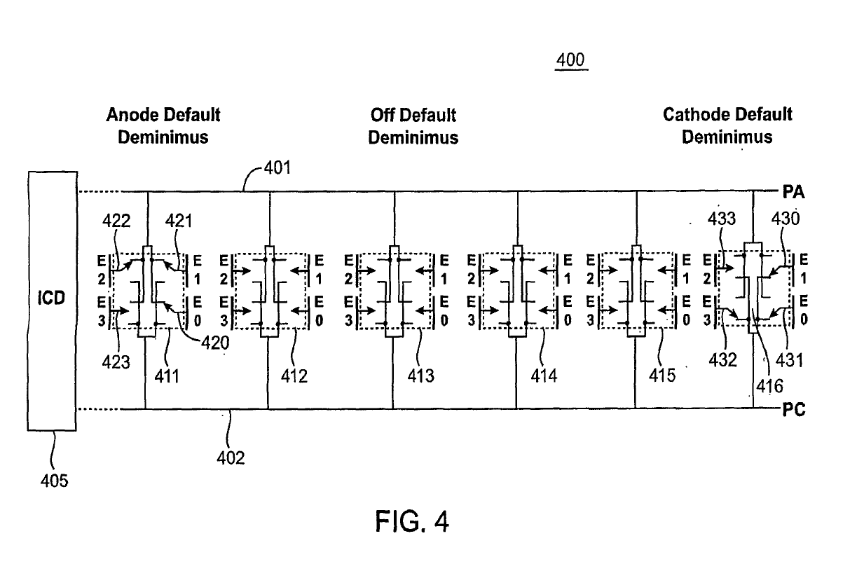

FIG. 4 illustrates an implantable pacemaker lead 400 according to a first embodiment of the present invention. Pacemaker lead 400 is coupled to pacemaker can 405 (ICD) through a connector (not shown) such as, e.g., an IS1 connector. Pacemaker lead 400 includes an anode wire 401 and cathode wire 402. When pacemaker can 405 is coupled to lead 400, current can flow from can 405 into anode wire 401 and back through cathode wire 402 to can 405.

Pacemaker lead 400 also includes multiple integrated circuit chips, such as chips 411-416. Each of the chips includes a set of four switches. For example, chip 411 has four switches 420-423. The switches are typically implemented by a set of transistors, which may have any convenient configuration.

Each of the switches in chips 411-416 is coupled to an electrode. For example, switch 420 is coupled to electrode E0, switch 421 is coupled to electrode E1, switch 422 is coupled to electrode E2, and switch 423 is coupled to electrode E3. A pacemaker lead of the present invention can have integrated circuit chips with any number of switches that are coupled to a corresponding number of electrodes. The four switches and four electrodes per chip that are shown in FIG. 4 are not intended to be limiting and are merely shown as an example.

The switches in each chip, such as switches 420-423, can be switched to couple a corresponding electrode to anode wire 401 or cathode wire 402. The switches can also decouple a corresponding electrode from both the anode wire 401 and the cathode wire 402 so that pacemaker can 405 cannot send current to that electrode. Thus, each of the switches can be placed in one of three states, decoupled, coupled to the anode wire 401, or coupled to the cathode wire 402.

FIG. 4 illustrates a pacemaker lead that enters a functional default mode after the supply voltage reaches a threshold voltage. The states of the switches illustrated in FIG. 4 are the default states of the switches of a pacemaker lead, according to one embodiment of the present invention. The switches enter the states shown in FIG. 4 after the supply voltage reaches the threshold voltage. In the particular default states illustrated in FIG. 4, each of the switches in chips 412-415 decouples its corresponding electrode from the anode wire 401 and the cathode wire 402. Thus, in the default mode shown in FIG. 4, none of the electrodes coupled to chips 412-415 can be charged by pacemaker can 405.

In the embodiment of FIG. 4, the switches in chips 411-416 decouple the electrodes from the anode wire 401 and the cathode wire 402 when the supply voltage is below a threshold voltage, by causing the switches to be in a high impedance state. When the supply voltage reaches the threshold voltage, lead 400 enters default mode. In the default mode, switches 420-422 in chip 111 couple electrodes E0-E2 to anode wire 401, switch 423 decouples electrode E3 from both wires 401-402, switches 430-432 in chip 416 couple electrodes E0-E2 to cathode wire 402, and switch 433 decouples electrode E3 from both wires 401-402. Thus, a pacemaker can 405 is able to stimulate cardiac tissue by sending current through the electrodes E0-E2 that are coupled to chips 411 and 416 in the default mode. The default configuration of the switches is maintained as long as the power supply requirements are satisfied.

A pacemaker lead of the present invention can interact with a lead impedance measurement function of a pacemaker can. In the embodiment of FIG. 4, the lead impedance measurement functions are only valid while the chips are successfully powered up. Depending on the lead impedance pass/fail criteria, the default chip might pass or fail. Lead impedance measurement functions can incorporate the correct pass/fail values for the default chips. The lead impedance can be, for example, in the range of about 40-720 Ohms.

The pacemaker lead of FIG. 4 is versatile enough to function in the default mode in response to a range of signals from a pacemaker can. According to some embodiments of the present invention, the default mode pacemaker lead of FIG. 4 can have a set of minimum signal requirements for responding to pacing pulses. For example, the default mode pacemaker lead may require minimum pacing pulse amplitude of about 2.0 volts (or 1.5 volts), a minimum pacing pulse of about 100 microseconds, and a minimum pulse interval of about 12 seconds.

The pacemaker lead of FIG. 4 can respond to bipolar pacing pulses in a default mode to stimulate cardiac tissue and provide an adequate charge balance that preserves the integrity of the electrodes. Alternatively, the pacemaker lead of FIG. 4 can operate in a unipolar mode. The pacemaker lead of FIG. 4 can also sense intra cardiac electrogram signals, IEGM, from cardiac tissue.

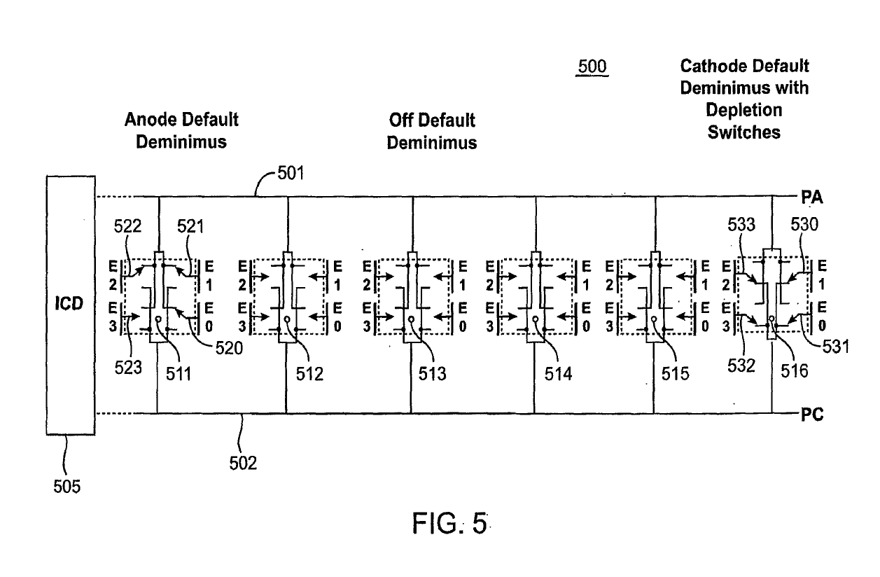

In a second embodiment of the present invention, FIG. 5 illustrates an implantable pacemaker lead. Pacemaker lead 500 is coupled to pacemaker can 505 (ICD) through a connector (not shown), e.g., an IS1 connector. Pacemaker lead 500 includes anode wire 501 and cathode wire 502. When pacemaker can 505 is coupled to lead 500, current can flow from can 505 into anode wire 501 and back through cathode wire 502 to can 505 in a bipolar mode. Similarly, when pacemaker can 505 is coupled to lead 500, current can flow from can 505 through tissue and cathode wire 502 to can 505, bypassing anode wire 501, in a unipolar mode. The ability to automatically operate in a unipolar mode is one of the distinguishing features of this embodiment.

In the embodiment of FIG. 5, switch 516 functions as a high performance cathode band. A high performance cathode band is a cathode with low impedance (e.g., in the range of about 30-60 Ohms at 0.2 volts). Switch 511 functions as a lower performance anode band. A lower performance anode band is an anode with higher impedance (e.g., about 360 Ohms at 2 volts). Switches 512-515 are off and have no function during default mode, although they can be turned on by can 505 in a non-default mode.

A pacemaker lead of the present invention can have integrated circuit chips with any number of switches that are coupled to a corresponding number of electrodes. Each of the switches in chips 511-516 is coupled to an electrode. For example, switch 520 is coupled to electrode E0, switch 521 is coupled to electrode E1, switch 522 is coupled to electrode E2, and switch 523 is coupled to electrode E3.

The switches in each chip, such as switches 520-523, can be switched to couple a corresponding electrode to anode wire 501 or cathode wire 502. The switches can also decouple a corresponding electrode from both the anode wire 501 and the cathode wire 502 so that the pacemaker can 505 cannot send current to that electrode. Thus, each of the switches can be placed in one of three states, decoupled, coupled to the anode wire 501, or coupled to the cathode wire 502.

According to some embodiments of the present invention, an implantable pacemaker lead is already in a functional default mode before the lead is coupled to a pacemaker can. FIG. 5 illustrates a pacemaker lead that is already in a functional default mode before the lead is coupled to a pacemaker can.

In the particular default states illustrated in FIG. 5, the switches in chips 512-515 decouple the electrodes from the anode wire 501 and the cathode wire 502 when the supply voltage is below a threshold voltage by causing the switches to be in a high impedance state. However, unlike the embodiment in FIG. 4, the embodiment in FIG. 5 does not require the supply voltage to reach a minimum threshold voltage before the pacemaker lead enters a functional default mode.

The transistors that form the switches in chip 516 are depletion transistors. Depletion transistors are transistors that have a low threshold voltage at, near, or below zero. Because depletion transistors have a low threshold voltage, they are on and able to conduct current without receiving a higher voltage at their gate terminals.

Because the switches in chip 516 are formed with depletion transistors, the switches in chip 516 turn on and couple the electrodes to cathode wire 502 before the supply voltage is powered up. Therefore, the embodiment in FIG. 5 is able to operate in unipolar mode without the supply voltage reaching a threshold voltage.

The switches in chip 511 contain transistors that have a higher threshold voltage than depletion transistors (e.g., enhancement transistors). Therefore, if bi-polar sampling is desired, the supply voltage of switch 511 must first reach a threshold voltage before the pacemaker lead enters a functional default mode during which bi-polar sampling is possible. Thus, pacemaker can 505 is able to stimulate cardiac tissue in unipolar or bipolar mode.

In the bipolar default mode, switches 520-522 in chip 511 couple electrodes E0-E2 to anode wire 501 and switch 523 decouples electrodes E3 from both wires 501-502. Switches 530-533, in chip 516 couple electrodes E0-E3 to cathode wire 502.

In bipolar mode, pacemaker can 505 is able to stimulate tissue by sending current through the electrodes that are coupled to chips 511 and 516 while in default mode. The default configuration of the switches is maintained as long as the power supply requirements are satisfied.

In unipolar mode, pacemaker can 505 is able to stimulate cardiac tissue by sending current from pacemaker can 505 through tissue and cathode wire 502 to can 505, bypassing anode wire 501. In the cathode default configuration, the switches are normally on and the electrodes are connected to the cathode. Thus, tissue can be captured without the supply voltage reaching a threshold voltage.

Unipolar sensing between can 505 or another lead (e.g., the anode wire) and cathode wire 502 in default mode can operate, for example, in the range of about 0.2V pacing. Similarly, bipolar voltage sampling between cathode wire 502 and any other lead or can in default mode does not require pacing to turn on the cathode band. Bipolar default pacing on cathode wire 502 is enabled when the supply voltage is at or near a predefined value, for example, about 2V.

The pacemaker lead of FIG. 5 can also interact with a lead impedance measurement function of pacemaker can 505. While in default mode using cathode wire 502 to can 505, lead impedance can be, for example, in the range of about 30-60 Ohm impedance to measurement assuming a simple, published measurement technique.

In another embodiment of the present invention, the default mode pacemaker lead of FIG. 5 operating in unipolar mode can deliver the lowest minimum pace pulse amplitude when pacing between cathode wire 502 and can 505.

The pacemaker lead of FIG. 5 can respond to bipolar and unipolar pacing pulses to stimulate cardiac tissue and provide an adequate charge balance that preserves the integrity of the electrodes. The pacemaker lead of FIG. 5 can also sense intra cardiac electrogram signals, IEGM, from cardiac tissue without requiring pacing.

According to another embodiment of the present invention, the pacemaker lead of FIG. 5 can be configured to eliminate capture issues present in prior embodiments. For example, depending on when bipolar pacing begins, the leakage current inside the chip is such that the transistors may no longer bias.

In one example of the present embodiment, a mechanism (not shown) is implemented to discharge the high voltage on the capacitors in a short time (e.g., about 30 seconds). One possible mechanism is a pacemaker can that can turn off the high voltage discharge feature. This feature allows for the ability to regulate switch impedance as pacing voltage increases in short time periods. In another example of the present invention, the leakage current is maintained high enough relative to the capacity of the storage capacitors such that the charge can be refreshed every few minutes.

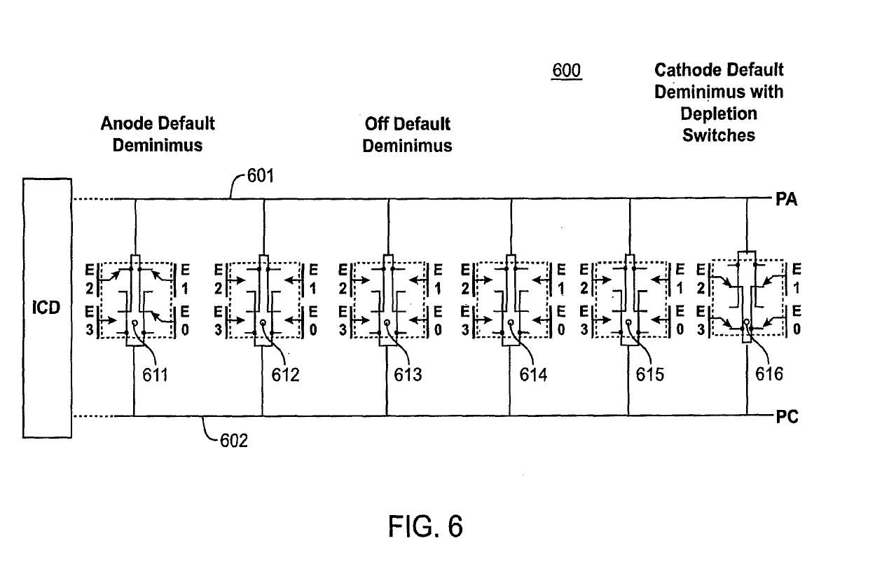

In another embodiment of the present invention, FIG. 6 illustrates an implantable pacemaker lead 600-Lead 600 includes an anode wire 601, a cathode wire 602, and chips 611-616. Lead 600 has the capability to eliminate capture issues present in prior embodiments. Unlike the embodiment of FIG. 5, the embodiment of FIG. 6 eliminates capture issues by placing depletion transistors at anode wire 601 as well as cathode wire 602. In one example of the present embodiment, pacemaker leads include depletion transistors both on the anode and cathode chips, 611 and 616 respectively. Thus, the embodiment of FIG. 6 is able to function in a bipolar default mode prior to receiving a power supply voltage.

An example of one such embodiment includes a depletion transistor placed between the electrode on the anode chip and anode lead 601. Similarly, a depletion transistor is placed between the electrode on the cathode chip and cathode lead 602. Placing depletion transistors both on the anode and cathode wires avoids the timing issue associated with transistors that are not properly biased because both anode and cathode chips turn on at low voltages.

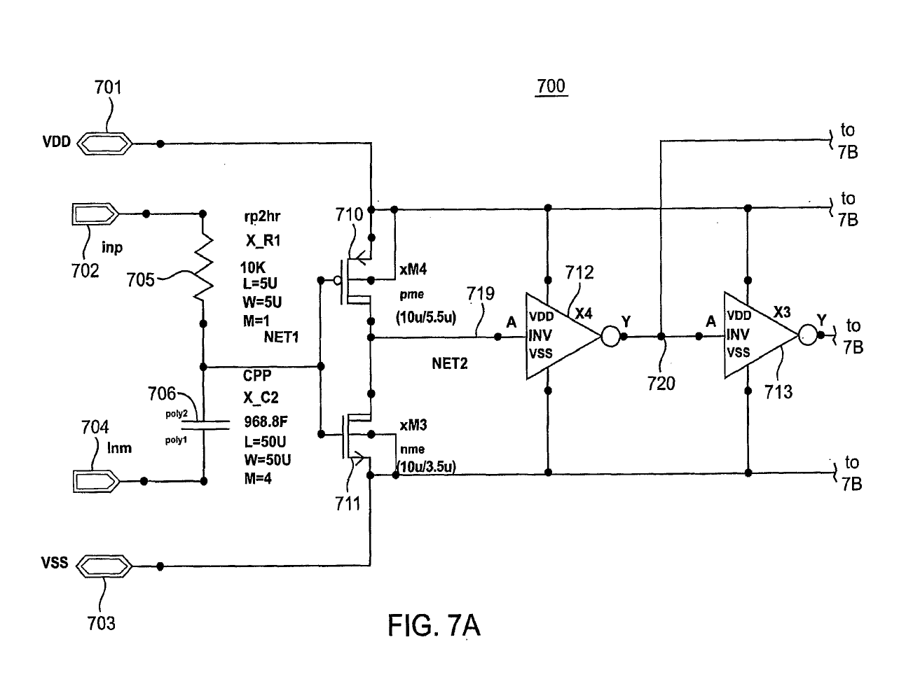

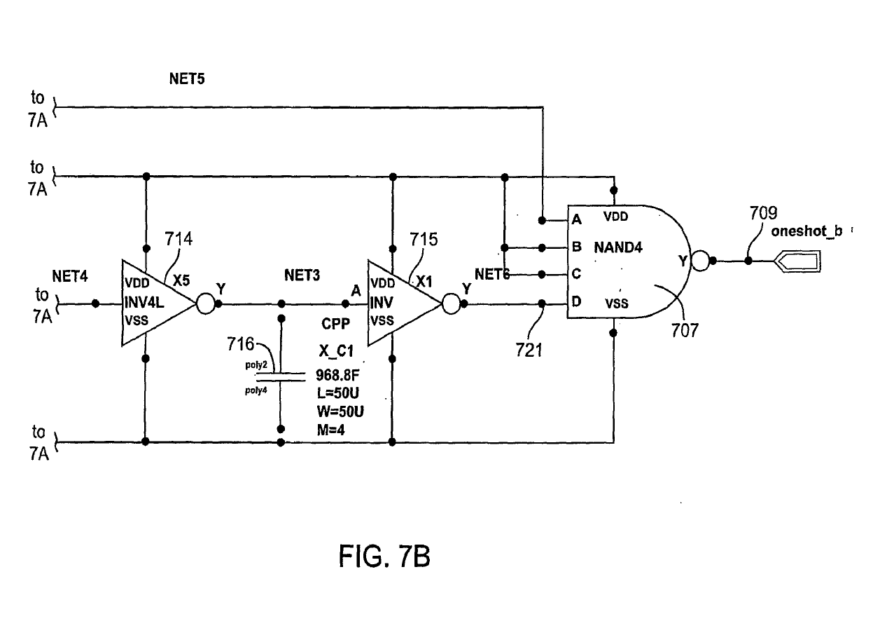

FIGS. 7A and 7B illustrate a one shot circuit 700 that initiates the default mode of operation in an implantable pacemaker lead, according to another embodiment of the present invention. One shot circuit 700 can be used in chips 411, 416, 511, and in other chips that cause one or more switches to enter a default state when the power supply voltage and a signal from the pacemaker can reach threshold values.

One shot circuit 700 includes resistor 705, capacitor 706, p-channel transistor 710, n-channel transistor 711, inverters 712-715, capacitor 716, NAND gate 707, and output terminal 709.

VDD 701 and VSS 703 represent the internal high and low power supply voltages within the circuitry. VDD 701 and VSS 703 can be powered by internal pacing of can 405. The voltage of VDD 701 depends on the pacing amplitude of can 405. For example, if can 405 is pacing at 2V, VDD 701 has a voltage of 1.4V.

In one embodiment of the present invention, can 405 begins by charging supply voltages VDD 701 and/or VSS 703. Power supply voltages 701 and 703 are provided to circuit elements 707 and 710-715. Supply voltage 701 is also provided to the B and C inputs of NAND gate 707. When the supply voltage at inputs B and C reaches a threshold of NAND gate 707, NAND gate 707 interprets the voltages as logic high signals.

Pacemaker can 405 provides signals to input terminals 702 and 704. Initially, the voltage difference between terminals 704 and 702 is zero, and the voltage at node 721 is a logic high. The output voltage of NAND gate 707 is a logic high, because the voltage at node 720 is a logic low. Pacemaker can 405 begins by increasing the voltage differential between terminals 704 and 702, causing capacitor 706 to charge up.

Transistors 710 and 711 form an inverter circuit. When the voltage difference between terminals 704 and 702 exceeds the threshold of inverter 710/711, the output 719 of the inverter 710/711 goes to a logic low. The RC circuit 7051706 creates a delay in causing the output of inverter 710/711 to change state. When output 719 is a logic low, inverter 712 pulls the voltage at node 720 to a logic high. The output terminal 709 of NAND gate 707 then falls to a logic low, because all of its inputs are at logic high states.

The output terminal 709 of NAND 707 remains low until the signal from node 720 propagates through inverters 713-715 to node 721. Specifically, after node 720 is pulled high, inverter 713 pulls its output low, causing inverter 714 to pull its output high, which causes inverter 715 to pull its output low. After the output 721 of inverter 715 is pulled to a logic low again, NAND gate 707 pulls output 709 back to a logic high.

Thus, one-shot circuit 700 generates a low voltage pulse at output terminal 709 in response to receiving a predefined voltage differential between input terminals 704 and 702. The duration of the low voltage pulse is set, in part, by the capacitance of capacitor 716. Capacitor 716 delays the passage of signals between node 720 and node 721. As a specific example that is not intended to be limiting, capacitor 716 can be 968.8 farads. The capacitance value can be changed to vary the duration of the low voltage pulse at output 709.

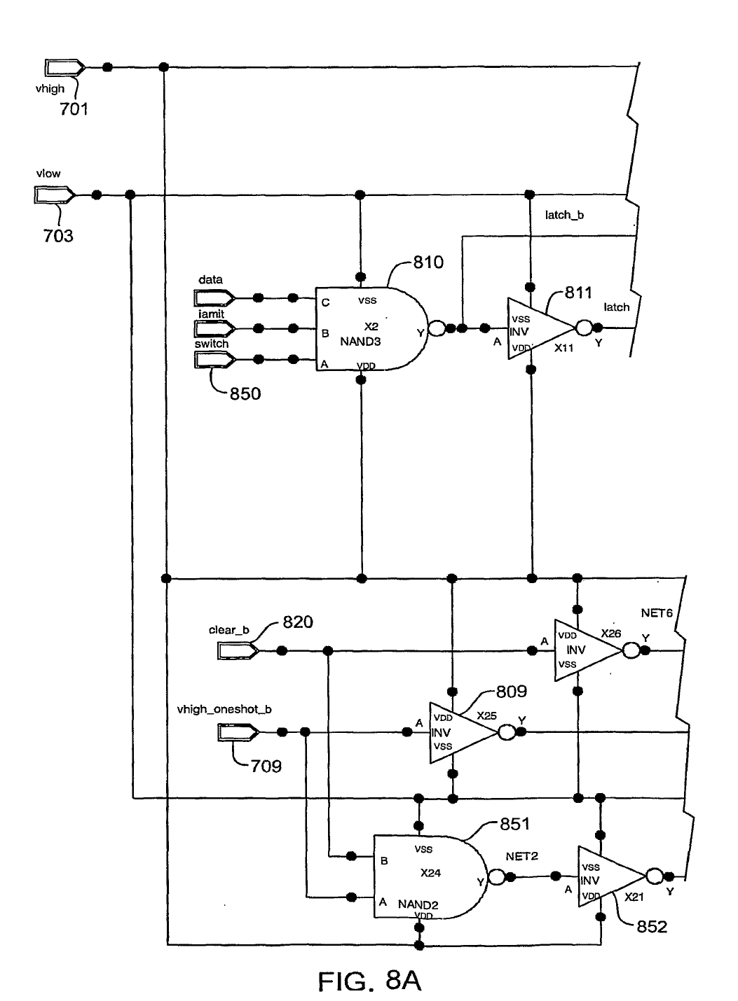

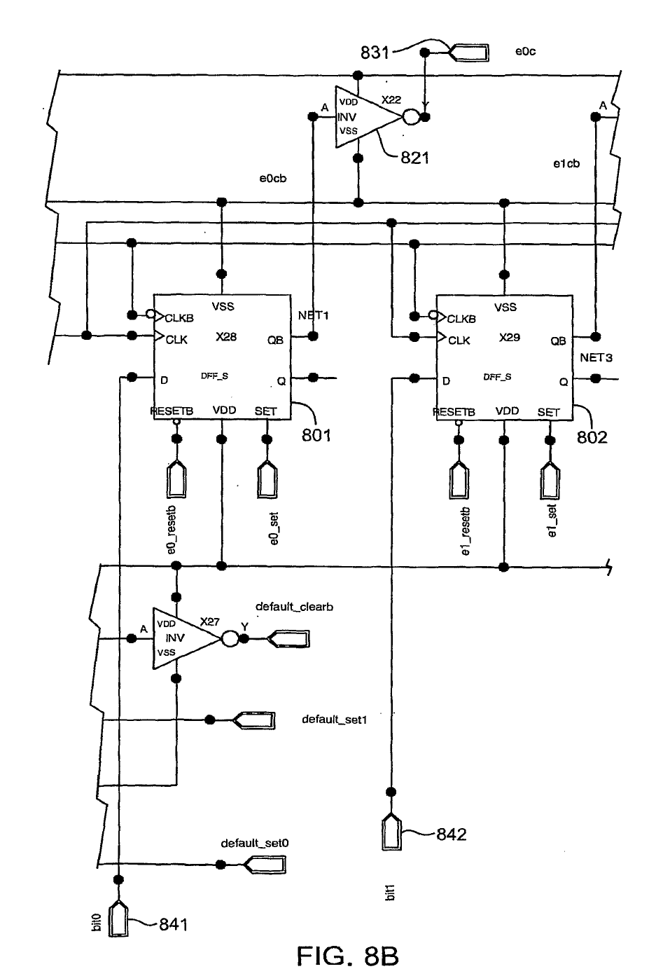

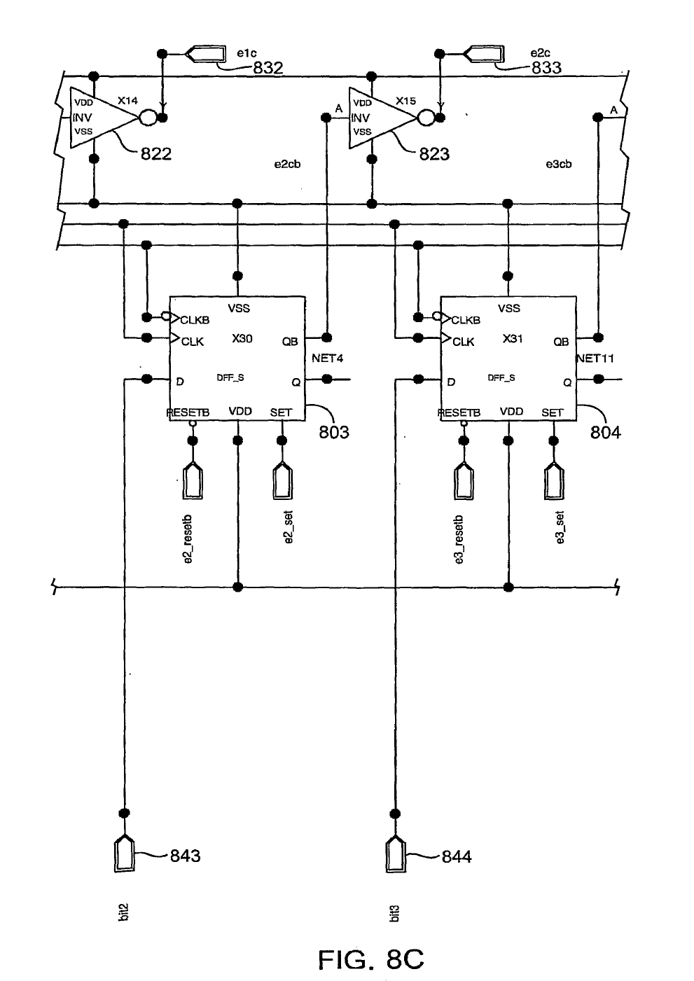

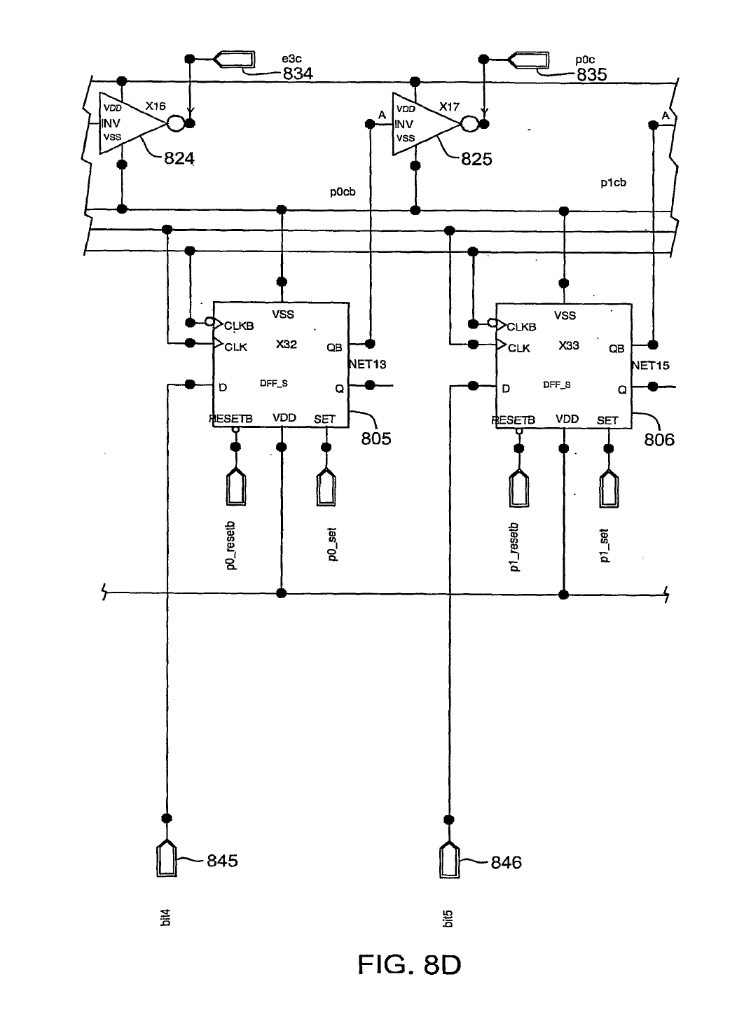

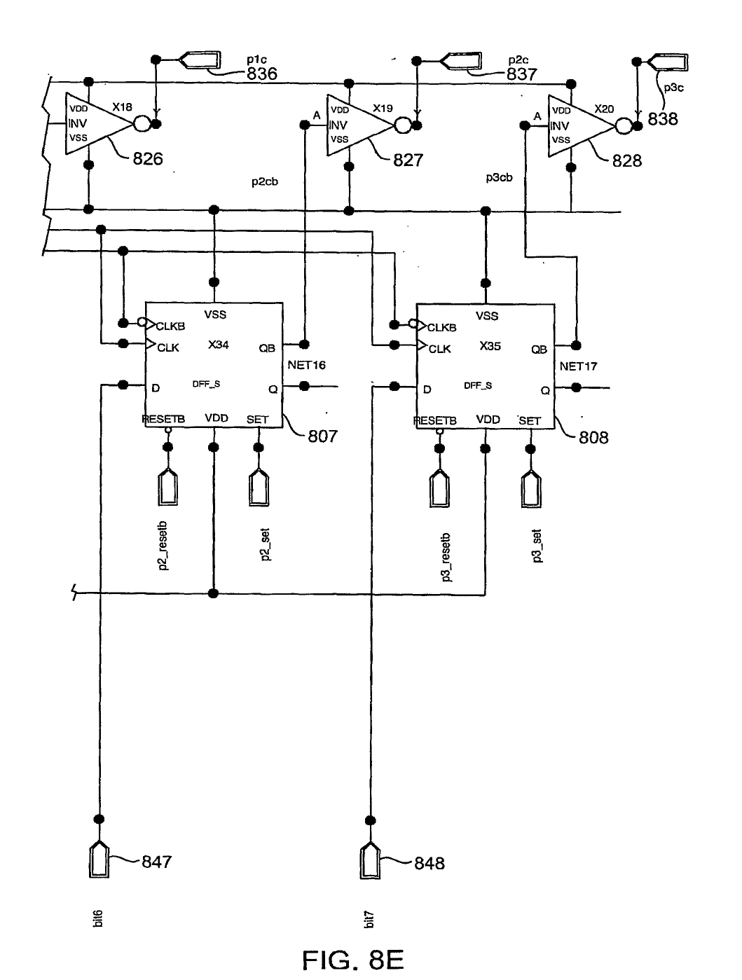

FIGS. 8A-E illustrate a register array according to another embodiment of the present invention. Integrated circuit chips, such as chips 411, 416 and 511, that cause their switches to enter default configurations in response to receiving a supply voltage at or above a threshold voltage can include register array 800. The threshold voltage is determined by the circuitry in one-shot 700. The register array controls the states of the switches in these chips.

The register array includes registers 801-808, NAND gates 810 and 851, and inverters 809, 811, 821-828, and 851. Each of the registers 801-808 has a clock input CLK, a clock bar input CLKB, a data input D, a reset input RESETB, a set input SET, a high supply voltage input VDD coupled to terminal 701, a low supply voltage input VSS coupled to terminal 703, and a Q bar output QB.

The clock CLK and clock bar CLKB inputs of the registers are controlled by a clock signal received at an input 850 of NAND gate 810. The CLK inputs are coupled to the output of inverter 811, and the CLKB inputs are coupled to the output of NAND gate 810.

The QB outputs of registers 801-808 are coupled to the inputs of inverters 821-828, respectively. The outputs of inverters 821-828 are coupled to output terminals 831-838, respectively. The voltages on output terminals 831-838 act as digital signals that control the states of four switches in a chip during a default mode. Because the switches described above with respect to FIGS. 4-6 can be in 3 different states, two digital signals are used to control the state of each switch.

In the embodiment of FIGS. 8A-E, the voltages at terminals 831-834 control whether the four switches are enabled or disabled in default mode. When a switch is disabled, it is decoupled from the anode and cathode wires. When a switch is enabled, it can be coupled to either the anode wire or the cathode wire. The voltages at terminals 835-838 control whether the four switches are coupled to the anode wire or the cathode wire in default mode, if the corresponding switch is enabled.

The RESETB inputs of registers 801, 802, 805, and 807 are coupled to receive a clear signal at terminal 820. The RESETB inputs of registers 803, 804, 806, and 808 are coupled to receive a clear signal from terminal 820 via NAND gate 851 and inverter 852. NAND gate 851 is also coupled to receive the one-shot signal at terminal 709. A logic low on terminal 820 causes the QB outputs of the registers to reset to logic high states. In response, inverters 821-828 pull the voltages at outputs 831-838 down to logic low states.

The register array receives an input signal from the output terminal 709 of one-shot circuit 700 at the input of inverter 809. Inverter 809 converts the low voltage pulse at terminal 709 into a high voltage pulse that begins with a rising edge and ends with a falling edge.

In the embodiment of FIGS. 8A-E, output terminal 709 is coupled to the SET inputs of registers 801, 802, 805, and 807. After the output of inverter 809 goes high, the QB outputs of registers 801, 802, 805, and 807 transition to logic lows, causing inverters 821, 822, 825; and 827 to pull outputs 831, 832, 835, and 837 to logic high states.

The low supply voltage at terminal 703 is provided to the SET inputs of registers 803, 804, 806, and 808, so that registers 803, 804, 806, and 808 are not set. Instead, a logic low on terminal 709 during the one-shot low voltage pulse resets registers 803, 804, 806, and 808 at their RESETB inputs (through NAND gate 851 and inverter 852), causing the QB outputs of registers 803, 804, 806, and 808 to reset to logic high states. In response, inverters 823, 824, 826, and 828 pull the voltages on output terminals 833, 834, 836, and 838 to logic low states.

Therefore, the two switches coupled to output terminals 831 and 832 are enabled, and the two switches coupled to output terminals 833 and 834 are disabled. The switch coupled to output terminals 831 and 835 is enabled and coupled to the anode wire, because output terminal 835 is high. The switch that is coupled to output terminals 832 and 836 is enabled and coupled to the cathode wire, because output terminal 836 is low.

The connections shown in FIG. 8A-E are merely one implementation of how four switches can be configured in a default configuration. Terminals 703 and 709 can be coupled to different combinations of the SET inputs of registers 801-808 to achieve different default mode configurations of the switches, according to additional embodiments of the present invention.

The data inputs D of registers 801-808 are coupled to data terminals 841-848, respectively. A pacemaker can is able to actively control the states of the switches in the chip during a non-default mode by controlling the voltages at data terminals 841-848. For example, a pacemaker 405 can enable the second switch by pulling the voltage at terminal 842 high, which causes the voltage at output terminal 832 to go high. The switch can be coupled to the anode wire by pulling the voltage at terminal 846 high, which causes the voltage at output terminal 836 to go high.

### Charge Balanced Operation

A MEL allows simultaneous pacing and signal monitoring, which facilitates more accurate and effective cardiac resynchronization therapies. The generation of any heart pacing stimulus, however, gives rise to charge accumulation in body tissues. Until the accumulated charges dissipate appreciably, sensing electrical activity can be difficult. Furthermore, unbalanced charge accumulation on the electrodes can accelerate the corrosion of electrodes, thereby significantly shortening the lifetime of the MEL. This charge build-up problem is especially pronounced if the same electrodes are used for both pacing and sensing. Reliable sensing remains difficult as long as the potential arising from the accumulated charges is significantly greater than that resulting from a heartbeat.

A common practice to mitigate this problem is to perform charge-balanced pacing. A charge-balanced pacing waveform typically includes two or more phases. In each phase the polarity of the pacing pulse is reversed, resulting in a reverse current in the tissue as compared with the previous phase. This polarity reverse causes the charges accumulated in the tissue to dissipate more rapidly.

However, the increased relative voltage swing and polarity change between the two pacing electrodes associated with charge-balanced pacing could cause the electrode-switching circuitry on a multi-electrode lead to malfunction. Hence, what is needed is an electrode-switching circuitry that can withstand the large voltage swings during charge-balanced pacing. The present invention provides inventive circuitry configurations that facilitate stable switching of electrodes in a multi-electrode lead during charge-balanced pacing.

In one embodiment, two switching modules are respectively situated between an electrode and two bus wires used for pacing. Each switching module includes two back-to-back NMOS transistors whose sources are coupled together. This configuration prevents the body diodes within each transistor from forming a short circuit when the voltage across the two bus wires is reversed.

Further embodiments of the present invention provide circuits for generating control signals which can remain sufficiently higher or lower than the voltage on either bus wire. These configurations ensure that the switching modules can be sufficiently turned on or off during the entire cycle of charge-balanced pacing.

Embodiments of the present invention provide an implantable electrode-switching circuitry within a multi-electrode lead that can withstand the large voltage swings during charge-balanced pacing. During charge-balanced pacing, the voltage applied across two electrodes can swing from about −10 V to +10 V. This large voltage swing can result in unwanted operations in conventional CMOS-based switching circuitry used to configure the connectivity pattern for the multiple electrodes. One embodiment provides a unique circuit which precludes the switching circuit from directly coupling to the wires carrying pacing voltages, thereby avoiding an unwanted short circuit formed between the pacing wires.

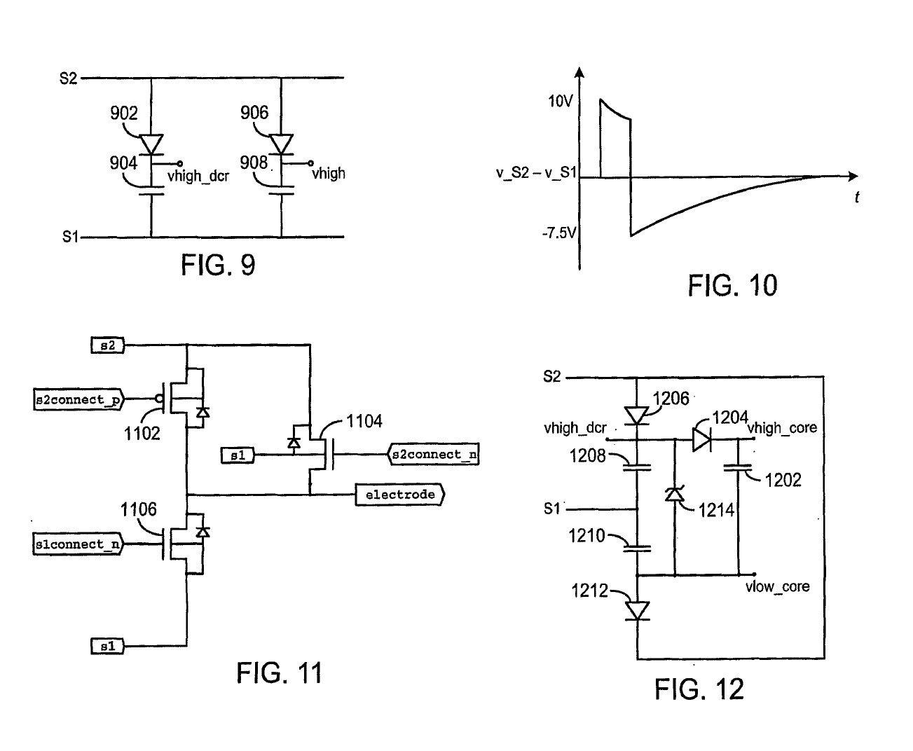

FIG. 9 is a high-level block diagram illustrating a simple power-supply circuit which could cause the electrode-switching circuit as is illustrated in FIG. 6 to malfunction during charge-balanced pacing. A high-voltage power supply, vhigh, is derived from the voltage on S2 using a diode 906 and a capacitor 908, assuming S2 carries a high voltage and S1 a low voltage during non-charge-balanced pacing. This power-supply voltage vhigh is used to drive several modules, including the CORE module and electrode-switching module.

However, during a reversed-polarity phase of charge-balanced pacing, the voltage difference between S2 and S1 can drop below the turn-on threshold of diode 906, causing diode 906 to be turned off. Power-supply voltage vhigh is hence provided solely by the charges stored in capacitor 908, which can dissipate more quickly than in the case of non-charge-balanced pacing where the voltage on S2 remains higher than the S1 voltage.

A worse problem could occur when the S2 voltage drops significantly below the S1 voltage. Such voltage reversion can cause MOS-based switches in the electrode switching module, to form unwanted short circuit. The description in conjunction with FIG. 11 describes such malfunction in further details.

A similar problem exists for the DCR high-voltage power supply, vhigh_dcr, which is derived from S2 and S1 voltages by using diode 902 and capacitor 904. When the voltage on S2 drops below the S1 voltage, diode 902 becomes reverse-biased, and vhigh_dcr is solely provided by the charges stored in capacitor 904, which can be discharged quickly. In general, the simple power-supply configuration as is illustrated in FIG. 9 cannot capture the power delivered in a pulse phase where S2 has a lower voltage than S1.

FIG. 10 is an exemplary voltage-waveform for a charge-balanced pacing cycling. A pacing cycle includes two phases. During the first phase, the S2 voltage starts at +10 V, while the S1 voltage is at 0 V. As time progresses, the S2 voltage drops slightly before the pacing cycle enters into the second phase where the S2−S1 polarity is reversed. At the beginning of phase two, S2 is at −7.5 V and S1 is at 0 V. Therefore, the voltage on S2 can swing from +10 V to −7.5 Vin a single pacing cycle. A power-supply circuitry using a conventional diode-capacitor configuration as is illustrated in FIG. 9 cannot capture the power delivered in the second phase of the pacing cycle.

FIG. 11 presents an exemplary electrode-switching circuit that could malfunction during charge-balanced pacing. This circuit is controlled by three control voltages, s2connect_p, s2connect_n, and s1connect_n. Assume that these three voltages are either at vhigh, as provided by a similar circuit as is illustrated in FIG. 9, or at the S1 voltage which is 0 V.

During a non-charge-balanced pacing cycle where S2 is at a higher voltage than S1, a low voltage at signal “s2connect_p” turns on PMOS transistor 1102 and couples the electrode to S2. Further, a high voltage at signal “s2connect_n” also turns on NMOS transistor 1104 and couples the electrode to S2 via NMOS transistor 1104. A high voltage at signal “s1connect_n” turns on NMOS transistor 1106 and couples the electrode to bus wire S1. Thus, the values (1, 0, 0), (0, 1, 0) and (1, 0, 1) for signal combination (“s2connect_p”, “s2connect_n”, s1connect_n″) can be used to decouple the electrode from S1 and S2, to couple the electrode to S2, and to couple the electrode to S1, respectively. Note that a logic “1” is at vhigh, and a logic “0” is at 0 V.

During a charge-balanced pacing cycle, when the S2 voltage drops below the S1 voltage, the body diodes at both NMOS transistor 1106 and PMOS transistor 1102 are forward biased. Consequently, a short circuit is formed through these two turned-on body diodes between S1 and S2. This short circuit prevents the pacing voltage from reaching the electrode and hence nullifies the reversed phase during a charge-balanced pacing cycle. Embodiments of the present invention provide unique circuit designs which allow the power-supply and electrode-switching circuitry to remain operative during both phases of a charge-balanced pacing cycle.

FIG. 12 is a schematic circuit diagram for a power supply circuit that provides three power-supply voltages, vhigh_core, vlow_core, and vhigh_dcr, in accordance with an embodiment of the present invention. This circuit derives a pair of high and low voltages for the CORE module, vhigh_core and vlow_core, from the voltage carried on S1 and S2. During operation, diode 1206 allows a high voltage on S2 to pass through and charge capacitor 1208. The high power-supply voltage for the DCR module, vhigh_dcr, is derived between diode 1206 and capacitor 1208.

When S2 is at a high voltage, capacitor 1202 becomes charged and provides the high power-supply voltage, vhigh_core, for the CORE module. Note that a Zener diode 1214 is used to limit the CORE power-supply voltages for the protection of CORE circuits. In addition, a diode 1204 reduces the leakage current through Zener diode 1214 which can discharge capacitor 1202.

Furthermore, the low CORE power-supply voltage vlow_core is derived from the voltage on S1 through capacitor 1210. A diode 1212 is used to allow vlow_core to be substantially at the S2 voltage when S2's voltage drops below S1's voltage.

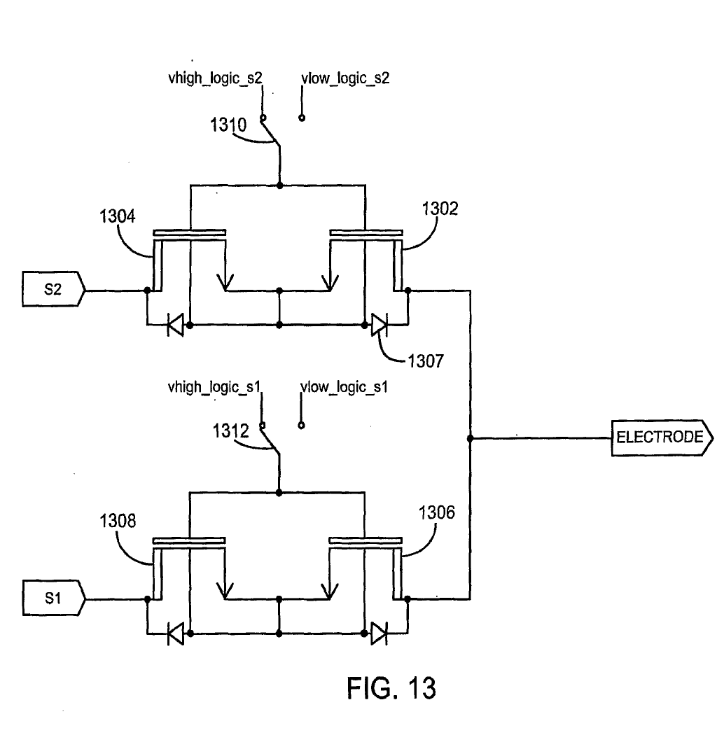

FIG. 13 is a schematic circuit diagram illustrating an electrode-switching circuit that can withstand large voltage swings and polarity changes during charge-balanced pacing, in accordance to one embodiment of the present invention. Two NMOS transistors 1304 and 1302, whose sources are coupled with each other, form a switch between the electrode and S2. The substrates of NMOS transistors 1302 and 1304 have the same potential, because generally an NMOS transistor's substrate forms a conductive path to the source and also forms a diode with the drain. The body diodes of transistors 1302 and 1304 are therefore coupled “back-to-back.” Hence, a conductive path cannot be formed from S2 to the electrode through the transistors' substrates, because the two body diodes cannot be turned on at the same time.

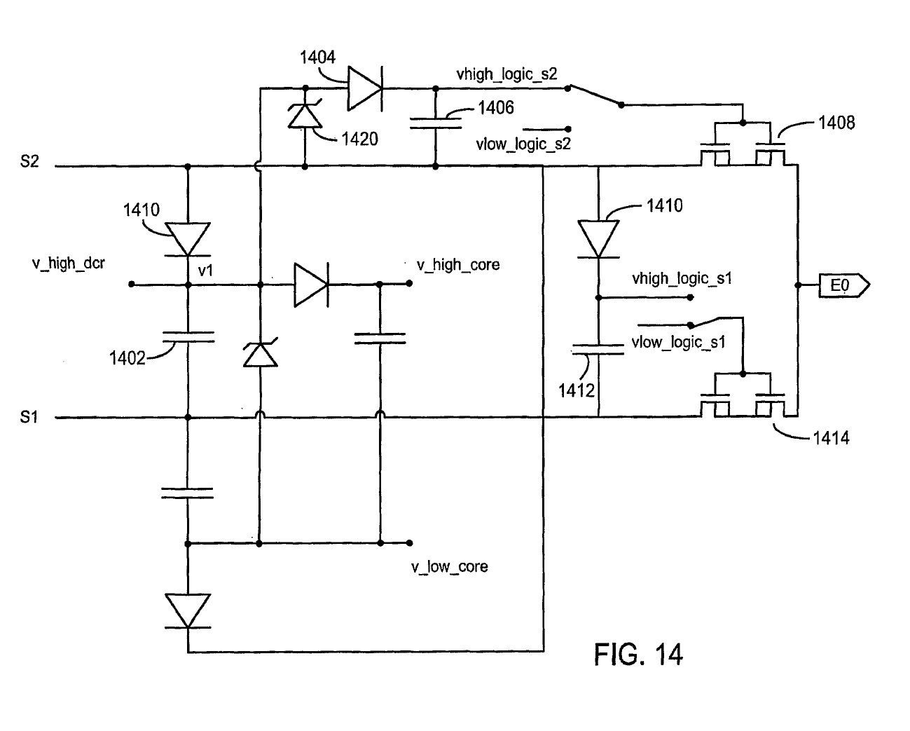

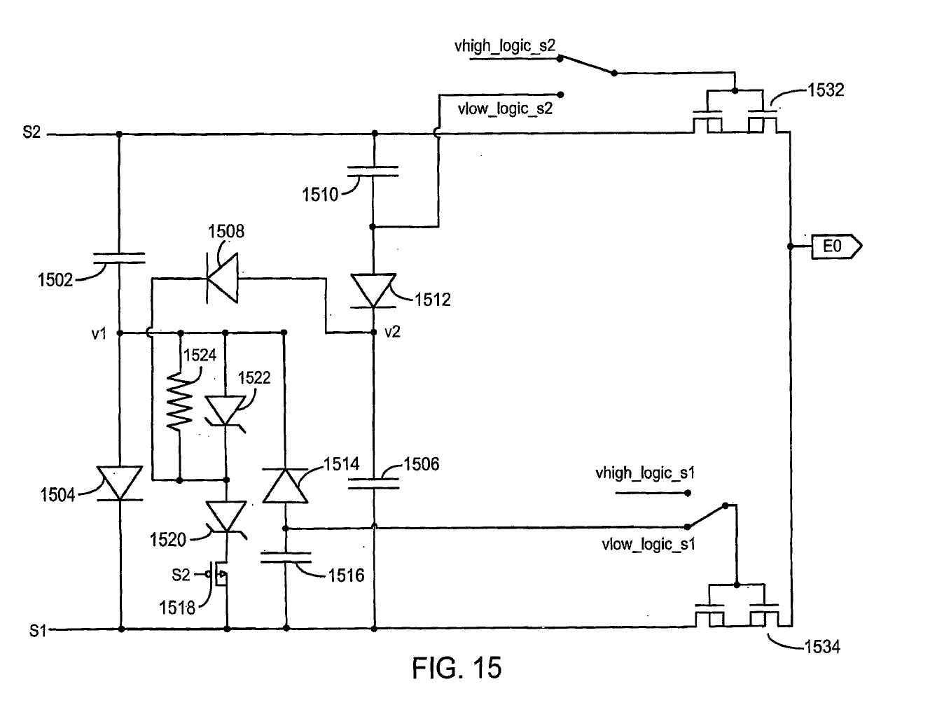

A switch 1310 and two control signals, vhigh_logic_s2 and vlow_logic_s2, controls whether the electrode couples to S2. vhigh_logic_s2 is a sufficiently high voltage which can fully turn on both NMOS transistors 1304 and 1302, and which can maintain its relative level when S2's voltage drops below S1's voltage. Correspondingly, vlow_logic_s2 is a sufficiently low voltage which can fully turn off both NMOS transistors during charge-balanced pacing. Note that switch 1310's state is controlled by the CORE module. The description in conjunction with FIGS. 14 and 15 provides more details on circuitry that provides vhigh_logic_s2 and vlow_logic_s2.

The S1-to-electrode switch employs a similar configuration. Two NMOS transistors 1308 and 1306, whose sources are coupled with each other, form a switch between the electrode and S1. The substrates of NMOS transistors 1308 and 1306 have the same potential. The parasitic body diodes 1307 of transistors 1308 and 1306 are therefore coupled “back-to-back.” Hence, a conductive path cannot be formed from S1 to the electrode through the transistors' substrates, because the two body diodes cannot be turned on at the same time.

A switch 1312 and two control signals, vhigh_logic_s1 and vlow_logic_s1, controls whether the electrode couples to S1. vhigh_logic_s1 is a sufficiently high voltage which can fully turn on both NMOS transistors 1308 and 1306, and which can maintain its relative level when S2's voltage drops below S1's voltage. Correspondingly, vlow_logic_s1 is a sufficiently low voltage which can fully turn off both NMOS transistors during charge-balanced pacing. Note that switch 1312's state is controlled by the CORE module. The description in conjunction with FIGS. 14 and 15 provides more details on circuitry that provides vhigh_logic_s1 and vlow_logic_s1.

FIG. 14 is a schematic circuit diagram illustrating a power-supply circuit that provides two switch control signals, vhigh_logic_S1 and vhigh_logic_S2, to the electrode-switching circuit as is illustrated in FIG. 13, in accordance to an embodiment of the present invention. Assume that the circuit is not energized before the first charge-balanced pacing cycle. During the first phase of the first cycle when S2 is at a high voltage, capacitor 1402 becomes charged. In addition, diode 1404 is off, causing vhigh_logic_S2 to be at substantially the same voltage as v1. Note that at this moment vhigh_logic_S2 may not be sufficiently high to completely turn on switch 1408. For instance, if S2 is at 4 V in this phase, vhigh_logic_S2 is also at about 4 V.

During the second phase when the S2 voltage drops to, say, −3 V, diode 1410 turns off, and the voltage maintained by capacitor 1402 turns on diode 1404. As a result, the charges accumulated in capacitor 1402 flow toward capacitor 1406 until diode 1404 turns off. Consequently, vhigh_logic_S2 remains substantially the same as v1, which can be about 4 V if the capacitance of capacitor 1402 is chosen to be significantly larger than the capacitance of capacitor 1406. Hence, capacitor 1406 holds about 7 V across, since vhigh_logic_S2 is at 4 V and S2 is at −3 V. vhigh_logic_S2 is therefore sufficiently high to turn on switch 1408 completely.

In the first phase of the next cycle when S2 is again at 4 V, capacitor 1406 maintains the 7 V voltage drop. Diode 1404 is then reversely biased, preventing capacitor 1406 from discharging. Consequently, vhigh_logic_S2 is at 11 V, which is significantly higher than the S2 voltage and can keep switch 1408 sufficiently turned on.

In the second phase of the same cycle, S2 voltage drops from 4 V to −3 V. v1 remains substantially at 4 V since diode 1410 is off. Because of the voltage drop on S2 and that the charges stored in capacitor 1406 are not discharged immediately, the 7 V voltage drop across capacitor 1406 is maintained, which results in vhigh_logic_S2 being at 4 V. Note that diode 1404 remains off because v1 and vhigh_logic_S2 are at about the same level. Hence, switch 1408 remains turned on.

Note that a Zener diode 1420 placed between the anode of diode 1404 and s2 prevents capacitor 1406 from being over-charged, limits the voltage of vhigh_logic_s2, and protects the transistors in switch 1408 from breaking down. In one embodiment, Zener diode 1420 has a break-down voltage of 5 V.

The process described above repeats during each charge-balanced pacing cycle. As a result, during the phase where S2 is at 4 V, vhigh_logic_S2 is at about 11 V; and during the phase where S2 is at −3 V, vhigh_logic_S2 is at about 4 V. Hence vhigh_logic_S2 remains sufficiently high to turn on switch 1408. Furthermore, the difference between vhigh_logic_S2 and the S2 voltage remains substantially constant during different pacing phases. The impedance of switch 1408 hence also remains substantially constant.

Note that the voltage values used in this example is only for illustration purposes. Embodiments of the present invention can be readily applied to other pacing voltage values. Furthermore, this circuit can also withstand pacing cycles with more than two phases.

vhigh_logic_S1 is derived using a conventional diode-capacitor configuration, since the gate voltages of the S1 switch 1414 only needs to be sufficiently higher than the S1 voltage. During the first phase of a pacing cycle when S2 is at a high voltage, diode 1411 is turned on, allowing capacitor. 1412 to be charged to about the same voltage as S2's voltage, which in this example is 4 V. During the second phase when the S2 voltage drops to −3 V, diode 1410 turns off, and vhigh_logic_S1 remains at about 4 V. Since S1 is assumed to be at 0 V during the entire pacing cycle, vhigh_logic_S1 can remain sufficiently high to turn on switch 1413.

FIG. 15 is a schematic circuit diagram illustrating a power-supply circuit that provides two switching voltages, vlow_logic_s1 and vlow_logic_s2, to the electrode-switching circuit as is illustrated in FIG. 13, in accordance to an embodiment of the present invention. Assume that the circuit is not energized before the first charge-balanced pacing cycle. During the first phase of the first cycle when S2 is at a high voltage, say, 6 V, diode 1504 turns on and charges capacitor 1502. As a result, v1 is at the same voltage as S1, which is at 0 V. Furthermore, diodes 1512, 1508, and 1504 are temporarily turned on until capacitor 1510 is charged with 6 V through resistor 1524, resulting in v2 and vlow_logic_s2 both being at 0 V after the circuit reaches equilibrium. Note that the transistors within switches 1532 and 1534 have finite break-down voltages, which in this example are assumed to be about 20 V. This circuit employs Zener diodes 1522 and 1520 to ensure that the switching voltages do not cause these switch transistors to break down. Further, assume that the Zener diodes 1522 and 1520 have a break-down voltage of 5 V. The operation of Zener diodes 1522 and 1520 is described in more details below.

During the second phase when S2 voltage drops to, say, −3 V, and capacitor 1502 which is initially charged with 6 V instantaneously draws v1 to −9 V. Consequently, diode 1504 turns off. Since v2 is initially at 0 V, diode 1508 turns on, draws v2 to −9 V, and charges capacitor 1506 through resistor 1524. Note that since Zener diode 1522 is not broken down, resistor 1524 provides a conductive path. After the circuit reaches equilibrium, capacitor 1510 holds 6 V across, and vlow_logic_s2 is at about −9 V, which is substantially lower than the S2 voltage and S1 voltage.

During the first phase of the next cycle when S2 voltage is at 6 V, diode 1508 is reversely biased and turned off because v2 is at −9 V and v1 is at 0 V. As a result, if capacitor 1510's capacitance is significantly larger than that of capacitor 1506, v2 and vlow_logic_s2 can both remain at about −9 V.

In the second phase of the same cycle, S2 voltage drops from 6 V to −3 V. Diode 1504 is reversely biased, and v1 is at −9 V. Since v2 is initially at about −9 V, diode 1508 is not turned on, and hence vlow_logic_s2 remains at about −9V.

The process described above repeats during each charge-balanced pacing cycle. As a result, vlow_logic_s2 remains significantly lower than the voltages on both S2 and S1, thus ensuring that switch 1532 remains sufficiently turned off.

vlow_logic_s1 is derived from capacitors 1502 and 1516, and diode 1514. During the first phase when S2 voltage is at 6 V, v1 and vlow_logic_s1 are both at 0V. During the second phase when S2 voltage drops to −3 V, v1 drops to −9 V since diode 1504 turns off. Consequently, diode 1514 turns on and negatively charges diode 1516, thereby drawing vlow_logic_s1 to −9 V.

When S2 voltage is at 6 V in the next pacing cycle, diode 1514 is turned off because v1 is at 0 V and vlow_logic_s1 is at −9 V. Therefore, vlow_logic_s1 can remain at −9 V during further pacing cycles.

Note that since Zener diodes 1522 and 1520's break-down voltages are 5 V, when coupled in series, these diodes jointly prevent capacitor 1516 from being charged for over 10 V and, as a result, prevents vlow_logic_μl from dropping more than 10 V below the S1 voltage. That is:

V(S1)−V(vlow_logic_{—}s1)<10 V (eq. 1)

In addition, PMOS transistor 1518 prevents current leakage, through Zener diodes 1520 and 1522 when S2 voltage is higher than S1 voltage. PMOS transistor 1518 is turned on only when S2 voltage drops below S1 voltage. Hence, so long as S2 voltage is higher than S1 voltage, the charges stored in capacitor 1502 can be maintained.

Zener diode 1520 also prevents capacitor 1506 from being charged for more than 5 V. That is:

V(S1)−V(v2)<5 V (eq. 2)

Further, assume that during the entire pacing process, the S2 voltage does not exceed 10 V above the S1 voltage:

V(S2)−V(S1)<10 V (eq. 3)

Combining eq. 2 and eq. 3 results in:

V(S2)−V(v2)<15 V (eq. 4)

Since v2 is substantially the same as vlow_logic_s2, we have:

V(S2)−V(vlow_logic_{—}s2)<15 V (eq. 5)

Since the maximum pacing voltage between S2 and S1 does not exceed 10 V, referring to the description in conjunction with FIG. 9, we have:

V(vhigh_logic_{—}s1)−V(S1)<10 V (eq. 6)

Further, because of Zener diode 920 which prevents capacitor 906 from being charged for over 5 V, the following condition is also true:

V(vhigh_logic_{—}s2)−V(S2)<5 V (eq. 7)

Combining eq. 5 and eq. 7 results in:

V(vhigh_logic_{—}s2)−V(vlow_logic_{—}s2)<20 V (eq. 8)

which protects the transistors within switch 1532 from being subject to a voltage switching greater than about 20 V.
Similarly, combining eq. 1 and eq. 6 results in:

V(vhigh_logic_{—}s1)−V(vlow_logic_{—}s1)<20 V (eq. 9)

Hence, according to eq. 8 and eq. 9, all the devices subject to vhigh_logic_s2 and vlow_logic_s2, or vhigh_logic_s1 and vlow_logic_s1, are protected from being subject to a voltage swing greater than 20 V.

Note that the voltage values used in this example is only for illustration purposes. Embodiments of the present invention can readily be applied to other pacing voltage values. Furthermore, this circuit can also withstand pacing cycles with more than two phases.

### Using Blocking Capacitors for Charge-Balanced Operation

As stated above, the reliable and robust operation of the MEL depends on charge-balanced operation during the pacing process. U.S. Pat. No. 4,903,700 describes a technique for achieving charge balance in a pacemaker. A typical pacemaker includes a coupling capacitor in the output circuit. Because the net current flow through a capacitor must be zero, the provision of AC coupling ensures that there is no net charge delivered to the body tissues. The output capacitor is generally part of the pulsing circuitry. Charge is stored on the capacitor, and it is then delivered rapidly over the lead when a stimulus is required. The charge delivered then flows in the opposite direction through the capacitor until the charges in body tissues are dissipated.

In order to speed up the charge neutralization, an “active” recharge circuit can be used to connect the output capacitor through a transistor switch to a potential source. This causes a larger reverse current to flow through the capacitor, and the charges stored in the body tissues dissipate more rapidly. Negative pulses are most often used to stimulate the heart. Thus, with an active recharge circuit, the pacing cycle consists of a negative pulse followed by a positive pulse.

However, complex circuitry is typically required to achieve a precise charge balance using this approach. Embodiments of the present invention provide an effective, but less complex, technique for achieving a precise charge balance in a pacemaker lead.

The present invention provides blocking capacitors in implanted pacemaker leads that can achieve charge balance. Each capacitor includes at least one conductive layer and a dielectric layer. According to some embodiments, a pacing electrode forms one of the conductive layers of the capacitor. A dielectric layer is formed on top of the electrode. A second conductive layer formed on the dielectric layer completes the capacitor. Alternatively, the second conductive layer is omitted, and tissue in the patient's body acts as a second conductive plate for the capacitor.

Capacitors of the present invention can also be completely separate from a pacing electrode. For example, a capacitor of the present invention can be coupled between an electrode and an integrated circuit chip in a pacemaker lead. According to other embodiments, the capacitors have irregular surfaces that increase their surface area and capacitance to allow for enough charge storage to achieve charge balance.

Other objects, features, and advantages of the present invention will become apparent upon consideration of the following detailed description and the accompanying drawings, in which like reference designations represent like features throughout the figures referenced below.

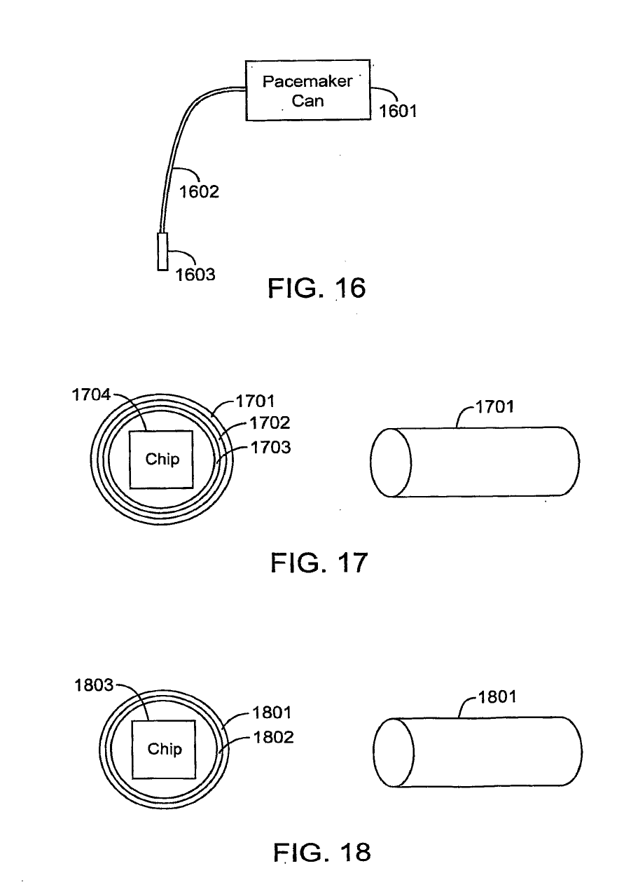

FIG. 16 illustrates an example of a pacemaker can 1601 that can be connected to an implantable pacemaker lead 1602. Pacemaker lead 1602 includes an electrode 1603 that is used to stimulate heart tissue. Can 1601 contains electronics that send pacing pulses along lead 1602 to electrode 1603.

FIG. 17 illustrates a blocking capacitor in a pacemaker lead, according to a first embodiment of the present invention. The lead contains an integrated circuit chip 1704 and an electrode. The electrode is formed by conductive layer 1703. Dielectric layer 1702 is formed on conductive layer 1703, and second conductive layer 1701 is formed on dielectric layer 1702. Layers 1701-1703 are formed in a cylindrical shape, and layer 1703 is electrically coupled to circuitry in chip 1704.

A capacitor is formed by conductive layer 1703, dielectric layer 1702, and second conductive layer 1701. Thus, the electrode forms an integral part of the capacitor of FIG. 17. Because the capacitor formed by layers 1701-1703 is on the distal end of the lead relative to the pacemaker can, the capacitor is able to provide charge balance to the pacemaker lead. Thus, the capacitor acts as a blocking capacitor that prevents charge build-up in the electrode.

Conductive layers 1701 and 1703 can be formed of metal, a conductive polymer, or another suitable type of conductive material. Dielectric layer 1702 can be formed of any suitable insulating material. Preferably, dielectric layer 1702 (and other dielectric layers of the present invention) are formed from a material that has a high dielectric constant. However, in some applications of the present invention, the dielectric layers can have a lower dielectric constant.

According to one specific example of FIG. 17, the capacitor has a diameter of 2 mm with a 300 Å thick dielectric layer having a dielectric constant of 3 to provide a capacitance of 11 nanofarads (nF). These numbers are provided merely as an example and are not intended to limit the scope of the present invention. Other embodiments of the present invention can provide a greater capacitance as described in detail below.

FIG. 18 illustrates a capacitor in a pacemaker lead, according to a second embodiment of the present invention. The lead of FIG. 18 contains an integrated circuit chip 1803 and an electrode. The electrode is formed by conductive layer 1802. Dielectric layer 1801 is formed on layer 1802. Layers 1801-1802 are formed in a cylindrical shape, and layer 1802 is electrically coupled to circuitry in chip 1803.

A capacitor is formed by conductive layer 1802, dielectric layer 1801, and a portion of the patient's cardiac tissue (not shown) that comes into contact with dielectric layer 1801. The electrode also forms an integral part of the capacitor of FIG. 18. The capacitor of FIG. 18 acts as a blocking capacitor that stores charge to provide charge balance to the pacemaker lead.

The embodiment of FIG. 18 can have a lower pacing threshold to stimulate the cardiac tissue than other electrode structures. An electrode with a lower pacing threshold requires a shorter pacing pulse, which reduces the amount of capacitance that is needed to provide an adequate charge balance.

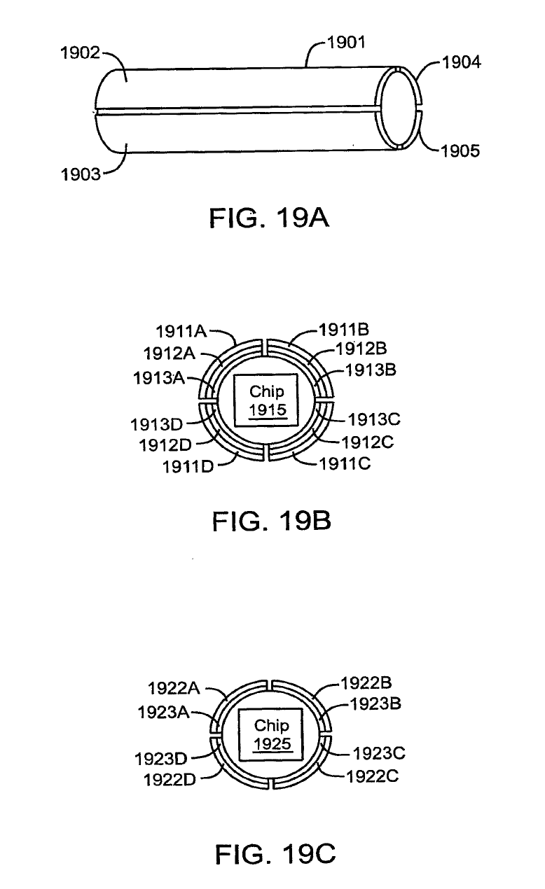

FIGS. 19A-19C illustrate four blocking capacitors that are formed in a pacemaker lead with four quadrant electrodes, according to further embodiments of the present invention. Referring to FIG. 19A, lead 1901 includes four electrodes 1902, 1903, 1904, and 1905. The four electrodes 1902-1905 form a cylindrical shape around lead 1901. Each of the electrodes 1902-1905 are electrically isolated from each other. A pacemaker can is able to pace from one of the electrodes to another one of the electrodes (e.g., from a positive electrode to a negative electrode).

In FIG. 19B, each of four quadrant electrodes around a pacemaker lead forms a blocking capacitor that provides charge balance to the lead. FIG. 19B illustrates four electrodes that are formed by conductive layers 1913A-1913D. Each of the electrodes is electrically coupled to a chip 1915. The electrode conductive layers 1913A-1913D are covered by dielectric layers 1912A-1912D, respectively. The dielectric layers 1912A-1912D are covered by a second set of conductive layers 1911A-1911D.

A first capacitor is formed by layers 1911A, 1912A, and 1913A. A second capacitor is formed by layers 1911B, 1912B, and 1913B. A third capacitor is formed by layers 1911C, 1912C, and 1913C. And a fourth capacitor is formed by layers 1911D, 1912D, and 1913D. Each of the electrode layers 1913A-1913C forms an integral part of one of the capacitors.

The capacitors act as blocking capacitors that store charge to provide charge balance to the pacemaker lead.

In the embodiment of FIG. 19C, the second conductive layers shown in FIG. 19B are omitted. Conductive layers 1923A-1923D are four quadrant electrodes that form a cylindrical shape and are coupled to a chip 1925. Four capacitors are formed by conductive layers 1923A-1923D, dielectric layers 1922A-1922D, and sections of the surrounding cardiac tissue (not shown).

A first capacitor is formed by layers 1923A, 1922A, and a portion of the surrounding tissue. A second capacitor is formed by layers 1923B, 1922B, and a portion of the surrounding tissue. A third capacitor is formed by layers 1923C, 1922C, and a portion of the surrounding tissue. A fourth capacitor is formed by layers 1923D, 1922D, and a portion of the surrounding tissue. The four capacitors provide charge balance to the lead.

The cylindrical shapes of the capacitors shown in FIGS. 17-18 and 19A-19C are shown for illustrative purposes and are not intended to limit the scope of the present invention. The techniques of the present invention can be applied to capacitors that have any desirable shape and size.

Typically, each of the four quadrant electrodes of FIGS. 19A-19C is smaller than a fully cylindrical electrode. Also, the charge generated by each of the quadrant electrodes of FIGS. 19A-19C is driven through less tissue than a fully cylindrical electrode. Therefore, the pacing pulses that are driven through the leads of FIGS. 19A-19C encounter less resistance than in the embodiments of FIGS. 17-18, and as a result, the capacitors shown in FIGS. 19A-19C require less capacitance than in the embodiments of FIGS. 17-18.

Some pacemaker leads contain multiple integrated circuit chips. Each chip is typically referred to as a satellite. The embodiments of FIGS. 19A-19C involve multiple intra-satellite pacing electrodes, because there are multiple electrodes coupled to one chip. In general, embodiments of the present invention that have multiple intra-satellite pacing electrodes require a smaller blocking capacitor than embodiments that have a single electrode for each satellite device.

The four quadrant electrodes and capacitors of FIGS. 19A-19C are merely examples of present invention and are not intended to be limiting. The techniques of the present invention can also be applied to any number of multiple intra-satellite electrodes (e.g., 3 electrodes, 5 electrodes, etc.) per chip.

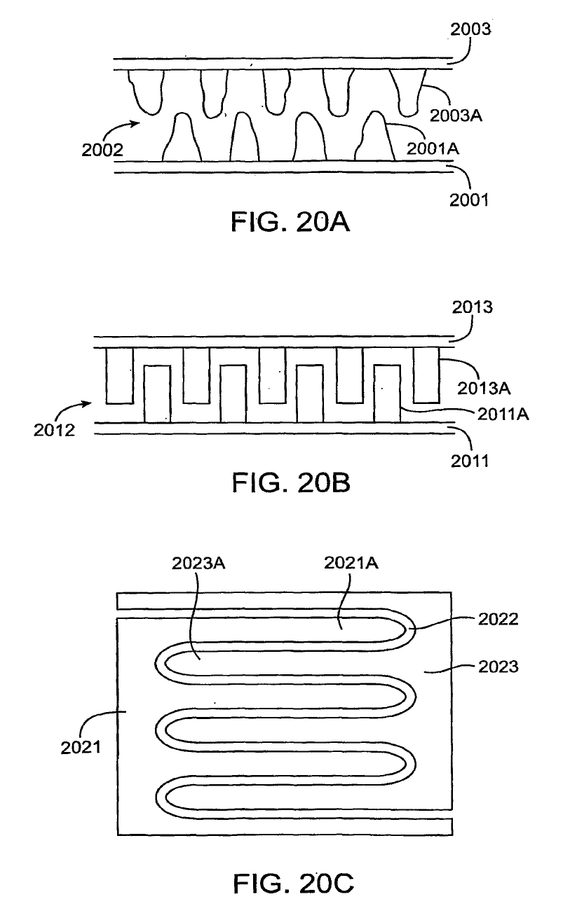

In some pacemaker leads, the capacitors shown FIGS. 17, 18, and 19A-19C may not have enough capacitance to provide a precise charge balance. FIGS. 20A-20C illustrate blocking capacitors in pacemaker leads with irregular (non-smooth) surfaces that provide for a greater capacitance, according to further embodiments of the present invention.

FIG. 20A illustrates a capacitor with a first conductive layer 2001, a dielectric layer 2002, and a second conductive layer 2003. The first conductive layer 2001 is an electrode for a pacemaker lead, e.g., as shown above in FIGS. 17, 18, and 19A-19C.

First conductive layer 2001 is formed with textured surfaces 2001A, and second conductive layer 2003 includes textured surfaces 2003A. Textured surfaces 2001A and 2003A increase the surface area of conductive layers 2001 and 2003, respectively, and thereby increase the capacitance of the capacitor.

Textured surfaces 2001A and 2003A can be formed, for example, using a standard electrode plating technique to form dendrite structures on the surface of a conductive layer. Textured surfaces 2001A and 2003A can also be formed, for example, with carbon nanotubes. Alternatively, textured surfaces 2001A and 2003 can be formed by micro patterning, e.g., using an etching technique or by seeding metal and causing selective growth.

As another example, textured surfaces 2001A and 2003A can be formed using a cathodic art that causes a porous surface to form using a plasma spray that contains large particles. Many other well known Capacitor formation techniques can be used with the principles of the present invention to form a capacitor having an irregular or bumpy surface.

FIG. 20B illustrates a capacitor with a first conductive layer 2011, a dielectric layer 2012, and a second conductive layer 2013. The first conductive layer 2011 is an electrode for a pacemaker lead, e.g., as shown above in FIGS. 17, 18, and 19A-19C.

First conductive layer 2011 is formed with fingers 2011A, and second conductive layer 2013 includes fingers 2013A. Fingers 2011A and 2013A increase the surface area of conductive layers 2011 and 2013, respectively, and thereby increase the capacitance of the capacitor.

Fingers 2011A can be formed by, for example, deposition and selective etching: Dielectric layer 2012 can then be formed by depositing or growing an insulating layer on top of layer 2011 that conforms to the irregular surface of layer 2011. Layer 2013 can then be formed or grown on top of layer 2012 such that fingers 2013A fill in the holes in dielectric layer 2012.

FIG. 20C illustrates a blocking capacitor includes a first conductive region 2021, a dielectric region 2022, and a second conductive region 2023. Either of conductive regions 2021 or 2023 can form an electrode. In FIGS. 20A and 20B, the structure is arranged in vertical layers. On the other hand, the capacitor of FIG. 20C is ordered horizontally in multiple layers. Conductive regions 2021 and 2023 have fingers (e.g., 2021A and 2023A) that are interleaved and separated by dielectric region 2022.

The blocking capacitors of FIGS. 20A-20C can provide a longer pacing pulse to a pacemaker lead than comparably sized, smooth surface capacitors, because the capacitors of FIGS. 20A-20C have a greater capacitance. For example, a 1 millisecond (ms) time constant for a pacemaker electrode with a body resistance of 1 kΩ (from the electrode to the can) implies a capacitance of 1 microfarad (μF). The non-smooth surfaces of the capacitors in FIGS. 20A-20C have a larger surface area that can increase the capacitance by a factor of, e.g., 100-400 to provide 1 μF of capacitance.

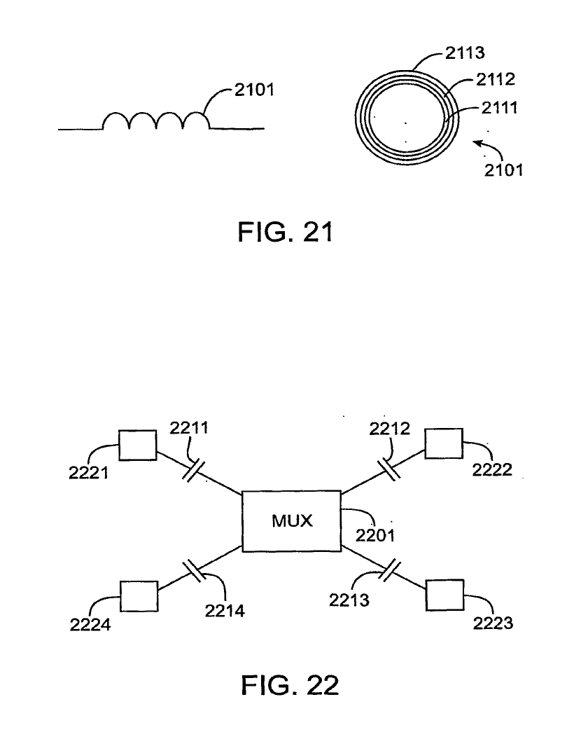

According to a further embodiment of the present invention shown in FIG. 21, a blocking capacitor 2101 that can provide charge balance in a pacemaker lead is formed on the surface of a helical screw-in electrode that is shaped like a corkscrew. A cross section of capacitor 2101 is shown on the right side of FIG. 21. The helical screw-in electrode is formed by first conductive layer 2111. Capacitor 2101 is formed by first conductive layer 2111, dielectric layer 2112, and second conductive layer 2113. Thus, the electrode 2111 is an integral part of the capacitor. According to a further embodiment of the present invention, second conductive layer 2113 can be omitted, and patient tissue can act as the second conductive layer of the blocking capacitor.

FIG. 22 illustrates another embodiment of electrodes that are not integral parts of blocking capacitors formed in a pacemaker lead. In FIG. 22, blocking capacitors 2211-2214 are formed in a pacemaker lead, according to an embodiment of the present invention. Capacitors 2211-2214 are coupled between multiplexer 2201 and electrodes 2221-2224. Multiplexer 2201 selectively couples pacing pulses from a pacemaker can to electrodes 2221-2224 through capacitors 2211-2214, respectively. Capacitors 2211-2214 provide charge balance to electrodes 2221-2224, respectively. Capacitors 2211-2214 can be formed on a single integrated circuit chip with multiplexer 2201 or on separate chips.

The present invention can include any blocking capacitor formed as a part of a pacemaker lead that is used to provide charge balance to the lead. The present invention includes capacitors having one conductive layer that forms a pacing electrode. The present invention also includes capacitors in a pacemaker lead that are completely separate from any pacing electrode.

The present invention also includes blocking capacitors in pacemaker leads that have one or more conductive layers covered with millions of tiny filaments called nanotubes. Each nanotube is very small (e.g., 30,000 times thinner than a human hair). The nanotube filaments on the capacitor increase the surface area of the conductive layers and allow the capacitor to store more energy. Such capacitors can be recharged many times (e.g., hundreds of thousands of times), and can be recharged very quickly.

The techniques of the present invention eliminate the need for a blocking capacitor in the pacemaker can. However, a blocking capacitor can be placed in the pacemaker can in addition to a blocking capacitor of the present invention.

### Multiplexer Circuits

Embodiments of the subject circuits may provide multiplexing functionality, e.g., as described below. While many advances have been made in pacemaker performance and capabilities, currently available pacemakers are not without their shortcomings. Many patients have implanted pacemakers which are not capable of interfacing and communicating with more than a limited number of electrodes or sensors. Particularly when a large number of effectors, whether for pacing or sensing or both, are employed, even with a pacemaker having multiplexing capabilities, changing the performance characteristics of the effectors or activating or deactivating effectors adds to the complexity of reprogramming a pacemaker.

Accordingly, it would be desirable to be able to easily and conveniently upgrade and adjust the functional parameters of currently available pacemakers. As such, it would be advantageous to provide a system which could be universally used to interface with and program or reprogram any brand of currently existing pacemakers.

The present invention provides modular circuits which are physically implantable adjacent to and electrically coupled between a pacemaker and the associated electrical leads. The modular circuits provide a communication link between the pacemaker and the plurality of electrodes and/or a plurality of sensors which are associated with the leads, and more particularly, communicates the input and output signals between the pacemaker and the electrodes and their associated electrode circuitry. More particularly, the multiplexing provides latches that can be controlled to connect to or disconnect from the pacemaker any of the electrodes associated with a given pacing lead. The subject circuits are able to maintain the various electrodes in their respective assigned state i.e., active or inactive, while minimizing leakage currents. In addition to controlling electrodes and sensors implanted within the body, the subject circuits also function as a communication link to devices external to the patient's body.

These and other objects, advantages, and features of the invention will become apparent to those persons skilled in the art upon reading the details of the invention as more fully described below.

The present invention provides modular circuits which are physically implantable adjacent to and electrically coupled between a pacemaker and the associated pacing leads. The modular circuits provide a communication link between the pacemaker and the plurality of electrodes (both for pacing and sensing) which are associated with the pacing leads, and more particularly, communicates the input and output signals between the pacemaker and the electrodes and their associated electrode circuitry. More particularly, the multiplexing provides latches that can be controlled to connect to or disconnect from the pacemaker any of the electrodes associated with a given pacing lead. The subject circuits are able to maintain the various electrodes in their respective assigned state i.e., active or inactive, while minimizing leakage currents. As such the circuits of the present invention may be referred to as a “switching circuits” due to their role of switching the states of the pacing electrodes and turning sensing electrodes and other types of sensors on and off.

In addition to controlling electrodes and sensors implanted within the body, the subject circuit also functions as a communication link to devices external to the patient's body. Such devices include programmers which can remotely control and program the switching circuit with operating or functional parameters particularly designed for the patient. These operating parameters may include, but are not limited to, assignment of the electrode states, the pulse width, amplitude, polarity, duty cycle and duration of a pacing signal, the number of pulses per heart cycle, and the timing of the pulses delivered by the various active electrodes.

In addition to downloading information from a programming device, the switching circuit may also be configured to upload information such as sensing data collected and stored within a memory element of the switching circuit. Such sensing data may include, but is not limited to, blood pressure, blood volume, blood flow velocity, blood oxygen concentration, blood carbon dioxide concentration, wall stress, wall thickness, force, electric charge, electric current and electric conductivity.

The switching circuit may also be capable of storing and transmitting data, i.e., cardiac performance parameters, which are calculated by it or the pacemaker from the sensed data. Such cardiac performance parameters may include, but are not limited to, ejection fraction, cardiac output, cardiac index, stroke volume, stroke volume index, pressure reserve, volume reserve, cardiac reserve, cardiac reserve index, stroke reserve index, myocardial work, myocardial work index, myocardial reserve, myocardial reserve index, stroke work, stroke work index, stroke work reserve, stroke work reserve index, systolic ejection period, stroke power, stroke power reserve, stroke power reserve index, myocardial power; myocardial power index, myocardial power reserve, myocardial power reserve index, myocardial power requirement, ejection contractility, cardiac efficiency, cardiac amplification, valvular gradient, valvular gradient reserve, valvular area, valvular area reserve, valvular regurgitation, valvular regurgitation reserve, a pattern of electrical emission by the heart, and a ratio of carbon dioxide to oxygen within the blood.

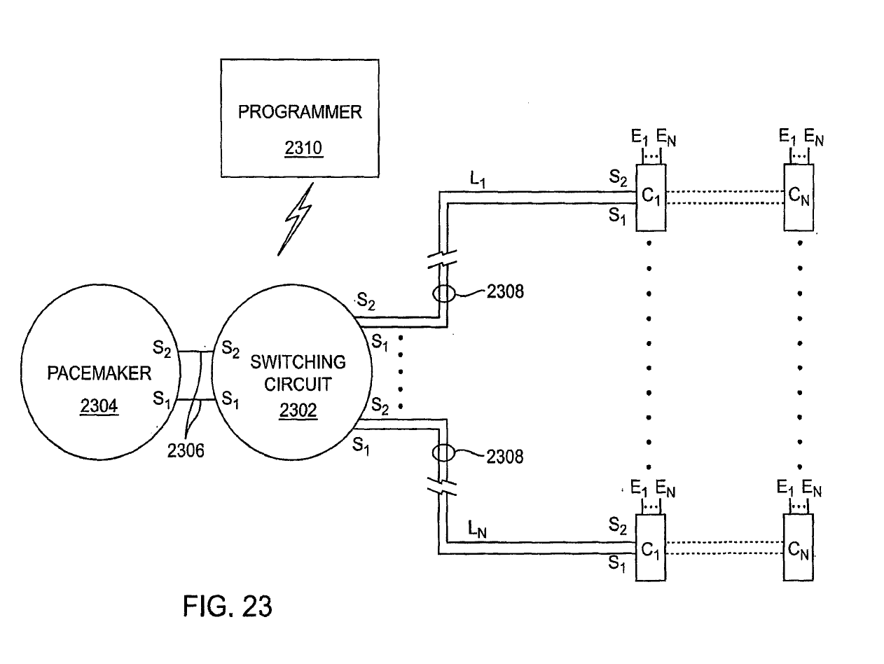

Referring now to the figures and to FIG. 23 in particular, there is shown a schematic representation of a switching circuit or “box” 2302 of the present invention operatively interfaced between and electrically coupled to a pacemaker 2304 (commonly referred to as a pacemaker “can”), which may be any conventional pacemaker, and a plurality of electrical leads L1-LN configured for placement within the heart in an arrangement and by procedures well known by those skilled in the art. Switching circuit 2302 may be housed within a “can” similar to that of pacemaker 2304 which housing is configured for implantation in the patient adjacent to pacemaker 2304.

Switching box 2302 is electrically coupled to pacemaker 2304 via a pair of signal lines 2306 which are referenced herein as S1 and S2, wherein S1 represents ground and S2 is the voltage supply. Lines 2306 may be configured at the pacemaker end in the form of a connector which can be plugged into standard pacemaker lead plug receptors.

As explained above, switching circuit 2302 multiplexes signals between the pacemaker 2304 and pacing electrodes, referenced as E1-EN, and their associated circuits C1-CN which provide the latching mechanisms, e.g., capacitors, for the electrodes. Each lead L1-LN then includes a ground line S1 and a voltage supply line S2, which signals are provided from pacemaker 2304 via switching circuit 2302. The number of electrodes per lead may vary from system to system and from application to application. A typical embodiment may provide four electrodes per circuit and eight circuits per lead L1-LN, where the total number electrodes per lead is thirty-two, for example. A multiple electrode lead allows for greater flexibility in lead placement as at least one of the four electrodes will be optimally positioned to pace the heart. Determining which of a lead's electrodes is best positioned to obtain or provide an accurate signal to and form the heart may be determined experimentally by controlled pacing of the heart and measuring the resulting threshold voltage of each electrode, wherein the electrode with the lowest threshold voltage is the most optimally positioned electrode for that lead.

Also, as mentioned above, switching circuit 2302 provides a communication link to external devices, such as programmer 2310, which can remotely control and program the switching circuit with operating or functional parameters, certain parameters of which can then be communicated to pacemaker 2304 by switching circuit 2302. While any mode of telemetry may be employed to transfer data between switching circuit 2302 and programmer 2310, one means suitable for use with implantable devices is electromagnetic coils, where one coil is provided in switching circuit 2302 and another is provided in programmer 2310. By placing the programmer in close proximity to the patient's chest in the vicinity of the implanted switching can, telemetric communication can be established. Information transmitted between the switching can and the programmer is in the form of AC signals which are converted into a corresponding DC voltage by the respective circuitry within each of the switching box 2302 and the programmer 2310.

The signal(s) transmitted by programmer 2310 and received by switching box 2302 provides a system operating current which powers up the circuit components, and further provides a series of commands for setting the system operating parameters identified above. Certain of these parameters are then transmitted to the electrode circuits C1-CN, via leads L1-LN. In particular, these parameters activate or deactivate according to a pre-selected configuration. Switching circuit 2302 then establishes the connections and enables communication between pacemaker. 2304 and the selected electrodes.

Switching circuit 2302 may provide certain other functions. While the latching capacitors of the electrode circuits C1-CN are intended to have very low leakage currents to recharge them as well as other capacitors utilized by the implanted system. Switching circuit may be configured to periodically supply a high voltage pulse for a few microseconds, possibly from about 10 to 20 microseconds, to recharge all the electrode and system capacitors. As such, if some current leakage has occurred, the charge across a capacitor is replaced thereby maintaining the electrodes in their currently “latched” condition. Additionally, switching circuit 2302 can be programmed to periodically, e.g., once daily, save the then current electrode status into memory. In case of a power glitch which disrupts the electrode status, switching circuit 2302 can reset the electrode capacitors to the last configuration stored in memory.

Another function which may be performed by switching circuit 2302 is that of transmitting analog signals from the electrodes to pacing can 2304. For example, where the pacemaker is attempting to sample voltages at a plurality of locations within the heart in order to generate a map of the heart's electrical potentials, switching circuit 2302 enables this by providing high-speed switching between the electrodes selected for the voltage sampling. More specifically, over a very short time period, on the order of milliseconds, the electrical potential at a selected electrode is sampled, the analog signal sent to pacemaker 2304, and repeated again. The faster the switching, the more accurate the “snap shot” of potentials is at various locations about the heart, and thus, the more accurate the electrical potential map.

The measured potentials are provided as analog signals which are carried from the electrodes to pacemaker 2304 by way of switching circuit 2302 where the signal from one electrode is provided on line S1 and the signal from another electrode is provided on line S2. An amplifier or voltage comparator circuit within pacemaker 2304 may then compare the two analog voltages signals. Based on this comparison, pacemaker 2304 will reconfigured the pacing parameters as necessary. While each electrode circuit may include an analog to digital converter which digitizes the analog voltage signal prior to sending it to switching circuit 2302, to do so would require larger electrode circuit chips. Not only would this latter configuration be more power consumptive, the time necessary for the electrode capacitor charges to settle and become balanced would be far greater.

Still yet, switching circuit 2302 may function as an analog to digital and digital to analog conversion system. A sensing protocol, either programmed within switching circuit 2302 or otherwise transmitted by an external program via switching system 2310, in the form of digital signals is converted to an AC signal by switching circuit 2302. These analog signals include current signals which drive sensing electrodes or other types of sensors, i.e., transducers, to enable them to measure physiological; chemical and mechanical signals, e.g., conductance signals, within the patient's body. The measured signals, also in analog form, are then converted to digital signals by the switching circuit 2302 and stored in memory, used to calculate other parameters by the switching circuit or transmitted to pacemaker 2304 and/or programmer 2310 for further processing.

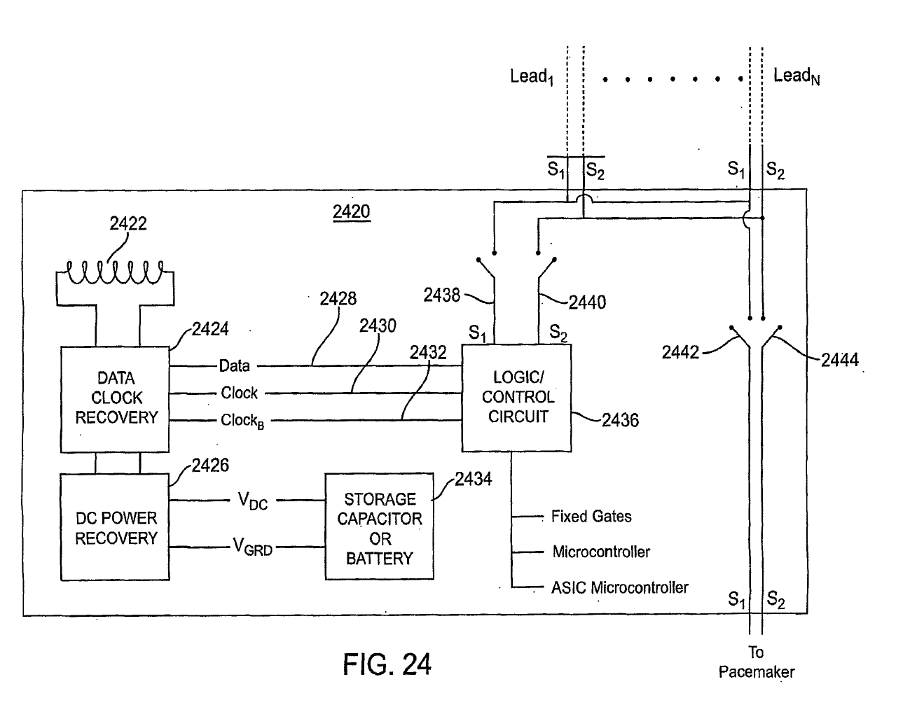

Referring now to FIGS. 24, 25 and 26, where like reference numbers refer to like elements, various embodiments of the switching circuit, there are provided schematic illustrations of various circuit embodiments of the switching circuit of the present invention. The switching circuit 2420 of FIG. 24 includes a coil 2422 that enables communication link to external devices, specifically, the transmission and reception of AC signals to and from a programmer, as described above. In addition to providing the pacemaker operating parameters, the received signals also provide power signals for operating the switch box as well as the electrode circuitry.

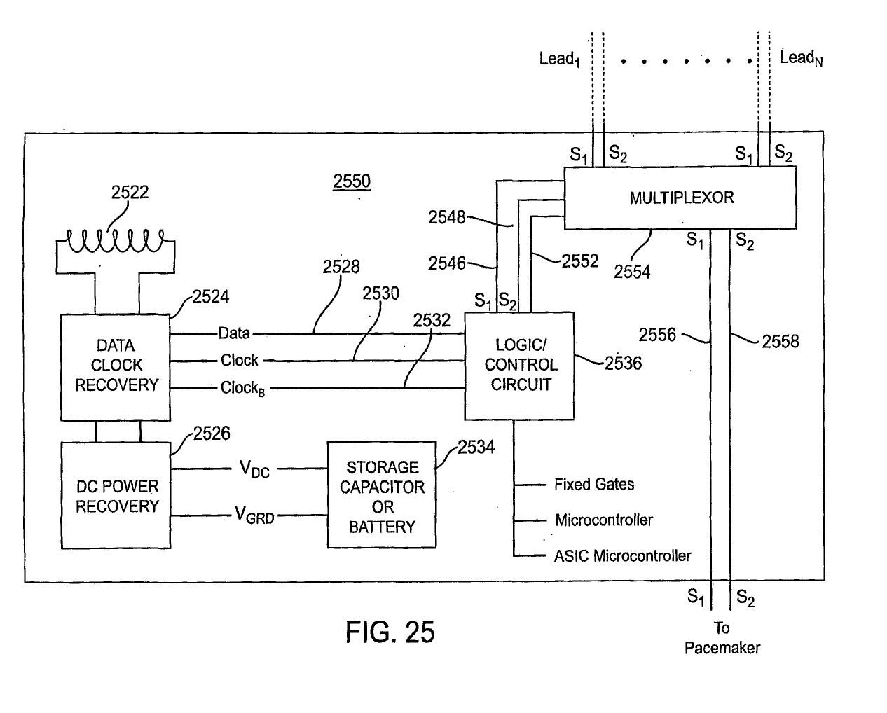

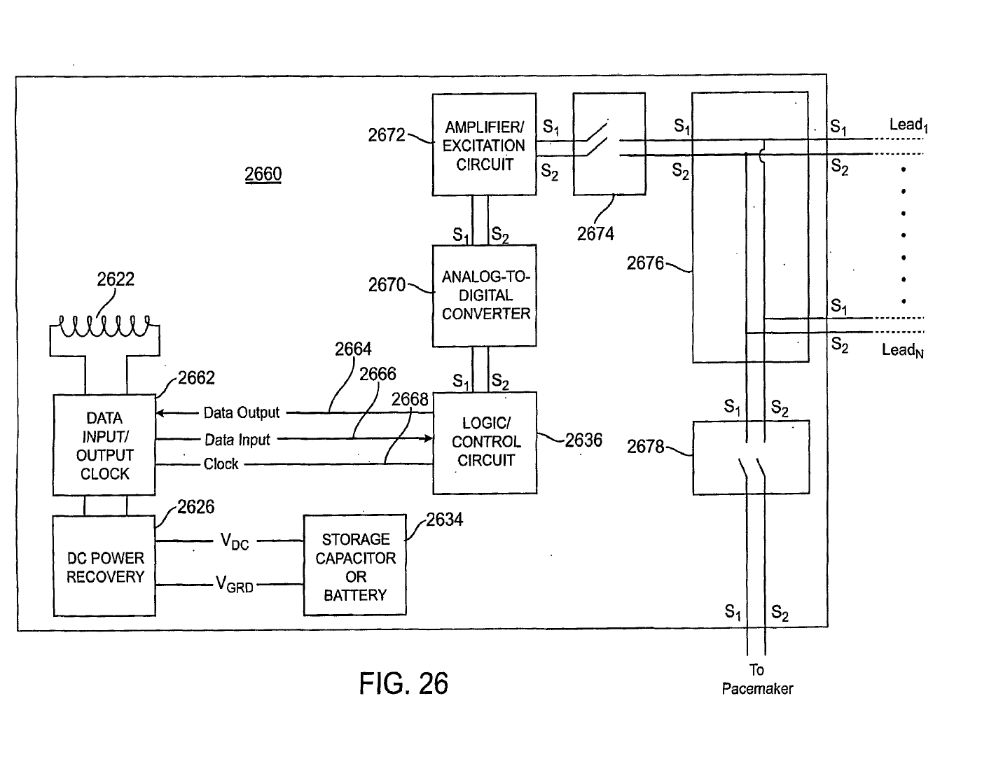

The signals transmitted by coil 2422 include cardiac performance characteristics as well as system status information. The coil's leads are connected to a data-clock-recovery circuit 2424 and a DC power recovery circuit 2426. DC power circuit 2426 produces a DC voltage VDC which is the supply voltage for the data-clock-recovery circuit 2424 and for a logic-control circuit 2440. Power circuit 2426 also supplies a ground voltage VGRD, which may be either a relative ground or a local ground. A capacitor 2434 is coupled between the supply and ground voltages, thereby functioning to store a charge which keeps power circuit 2426 and logic circuit 2440 powered after external power is discontinued, i.e., coil 2422 is turned off. The power portion of circuit 2420 just described with respect to the embodiment of FIG. 24 also applies to the embodiments of FIGS. 25 and 26, respectively.

Referring again to FIG. 24, data-clock-recovery circuit 2424 assesses the AC signal received by coil 2422 and extracts data and clock signals embedded therein. The clock signal is used to control the timing of data sent to and from logic and control circuit 2436. Many technologies are known in the electronic arts for performing the clock function. One such mechanism that may be used with the switching circuits of the present invention is frequency shift keying. Frequency shift keying changes the period of the received AC signal, e.g., a 1-MHz AC signal or a 1-msec signal period represents a one, and a 2-MHz AC signal or a 0.5-msec signal period represents a zero. Data signal 2428 and clock signal 2430 are then fed into logic and control circuit 2436.

Logic circuit 2436 controls the switching of the connections between lines S1 and S2 of each lead L1-LN with the various electrodes E1-EN through their associated circuits C1-CN. In other words, by way of switches 2438 and 2440, circuit 2436 selectively connects and disconnects the electrodes to and from lines S1 and S2 and, thus, to and from the pacemaker according to the switching protocol by which logic circuit 2436 is programmed. Logic circuit 2436 also controls the reprogramming of the electrode circuits and the implementation of the electrode switching protocol.

During reprogramming of the various electrode circuits, logic circuitry 2436 provides a ground signal on line S1 and a voltage signal on S2 representative of the switching protocol, which includes the timing at which the switches are made, i.e., clock signal, simultaneously to each of the leads L1-LN. Accordingly, each electrode circuit stores a charge on a very small capacitor, e.g., about a pico farad (1×10⁻¹²) capacitor. After reprogramming of the electrode circuits is completed, logic circuit 2436 is disconnected from the leads by opening switches 2438 and 2440, and the leads are connect to the pacemaker by closing switches 2442 and 2444. Logic circuit 2436 may include either a set of fixed gates, making it a relatively non-reprogrammable system, or be in the form of a standalone microcontroller or a microcontroller embedded into an ASIC.

FIG. 25 illustrates a switching circuit 2550 which is similar to switching circuit 2420 of FIG. 24 with the difference being that the signal lines S1 and S2 are multiplexed between the various leads L1-LN instead of being hardwired to them. This configuration is accomplished by multiplexer circuit 2554 coupled to signal input lines 2546 and 2548 from logic circuit 2536 and to output signal lines 2556 and 2558 which are in turn coupled to the pacemaker. This embodiment is particularly useful when employing large numbers of leads, for example, in a two-dimensional patch placed on the brain or when using multiple patches placed over the epicardium. Multiplexing allows selected leads or patches to be actively coupled to logic circuit 2536 and to the pacemaker while at the same time reducing power consumption (as well as leakage currents) by decoupling from the other leads or patches which are not currently selected.

FIG. 26 illustrates another switching circuit 2660 of the present invention which, in addition to controlling pacing electrodes on leads L1-LN, is also capable of controlling sensors (not shown) positioned on the leads. For example, these sensors may include strain gauge sensors that measure the bending of the lead, pressure sensors that measure the pressure inside the ventricle or a cardiac vein or other part of the heart or body, or electrical conductivity sensors that measure the conductivity between two pacing electrodes that are positioned relatively far apart in order to determine the distance between them. Other types of sensors which may be employed with switching circuit 2660 are described above.

Like the circuit of FIG. 25, switching circuit 2660 includes a multiplexer circuit 2676 which functions to multiplex between the plurality of pacing electrodes as described above but also serves to multiplex between various sensors. To facilitate collection and compilation of sensed data and to condition the signals representing the data for external transmission, an analog-to-digital converter 2670 and amplifier circuit 2672 are coupled between logic and control circuit 2636 and multiplexer 2676. Analog-to-digital converter 2670 receives the multiplexed sensor signals on lines S1 and S2 and converts the sensed analog signal (e.g., temperature, pressure, flow rate, etc.) to a digital value. The signal representing that digital value is then amplified and conditioned by amplifier 2672 for transmission to an external device. A switch 2678 at the output of multiplexer 2676 controls the transfer of signals to the pacemaker, while a switch 2674 is coupled between amplifier 2672 and multiplexer 2676 to control the direction of signals between the two, Another difference between circuit 2660 and the other switching circuits described is the provision of a designated data line 2664 for transferring data from logic and control circuit 2636 to data-and-clock circuit 2662, as well as a designated data line 2666 for transferring data from data-and-clock circuit 2662 to logic and control circuit. A clock line 2668 controls the timing of the transfer of both input and output data.

Thus, unlike the previously described one-way switching circuits, circuit 2660 is a duplex system which provides bi-directional transfer of signals, i.e., signals are sent to the body from switch box 2660 and other signals are received from the body (i.e., from sensors placed in the body). The bi-directionality of switching circuit 2660 provides the added advantage of being able to externally confirm that the appropriate instructions were received and transmitted to each of the electrode/sensor circuits.

Another advantage of the various switching circuits of the present invention is that they reduce the amount of power consumed and are thus able to function longer without be recharged. Specifically, the power consumption is kept to a minimum by deactivating components (e.g., electrode and sensor circuits) which are not necessary to the current function being performed by the pacemaker system. The unused components are put into a sleep mode and, as such, are not consuming power. As mentioned above, one way of accomplishing this by having a battery which provides a continuous charge to compensate for leakage currents of the various components. Alternatively, a capacitor is provided whereby a slight charge from the pacing pulses is used to replenish the capacitor, which eliminates the need for a battery.

The following embodiment of the invention refers to the Switching Circuit 2302 of FIG. 23. In this embodiment, the switching circuit is housed in an adapter that sits outside of the IPG but is electrically in parallel with S1 and S2 of the IPG. In this embodiment, the adapter is parasitic off of the pacing pulses provided by the IPG. Essentially, a small portion of power is taken from the pacing pulse to provide power to the Switch Circuit 2303 of FIG. 23. This power is used to run the electronics of the Switching Circuit. Since the sole purpose of these electronics is to maintain the settings on the switches in a certain way, there is no real communications power consumption. Rather, it is intended to maintain the device to the appropriate settings. One way of thinking of this is that every time the device sees a pacing pulse, it sends out the switch command, and that switch command is powered by the pacing pulse itself.

While these additional embodiments of the IPG interface device are similar to the broad concept shown in FIG. 23, there are several key differences. There are two different approaches to these embodiments of the interface device.

In the first interface device, the switching circuit 2302 is fixed so that it is not programmable. However, the interface device can still be used for a specific combination of electrodes that may be chosen at some time after installation of the device in a patient. Take the case where five years after the lead is installed, a proprietary pacemaker is used that does not have the imbedded switching circuit. Instead of having a programmable switching circuit, a fixed combination of satellite and electrodes is provided that is hardwired into Switching Circuit 2302.

Using this interface device configuration, every time the pacemaker paces a pulse, a switch command is sent out to reprogram the satellites to be in that configuration. The switch command may not need to occur every time the pacemaker pulses. By example, the reprogramming could occur every tenth time the pacemaker pulses. Alternatively, the reprogramming can occur after a set number of minutes. Other approaches to reprogramming may also be employed.

The switching circuit sends out a search command. It also sends out a refresh pulse. The result is that the charges on satellites C1-Cn are refreshed, and the electrodes are set appropriately.

In an additional version of this controller interface, the device is programmable during the replacement of the pacemaker. An example would be a switching circuit that is modified when the patient is being fitted with a new IPG or ICD. The Switching Circuit is programmed to activate a specific combination of satellites and electrodes. When the programmer is turned off, the settings are burned into the switching circuit 2302. The device continues to exercise its refresh and rewrite capability.

An advantage to these configurations lies in the simplicity of the device. For example, the switching circuit 2302 need not have a battery; it might have a storage capacitor. FIG. 24 provides a coil showing data clock recovery, and a DC power recovery.

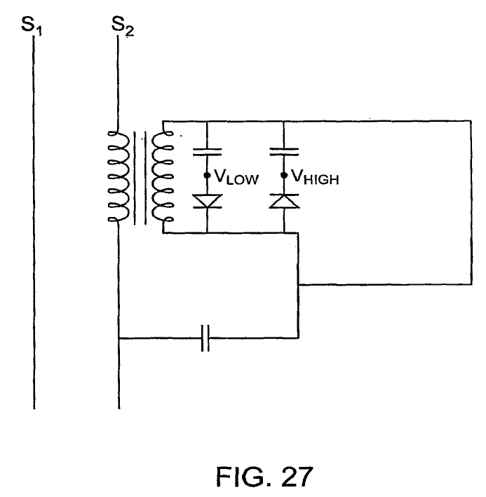

FIG. 27 provides another variation of this configuration. The pacing comes in at S2, and goes through a coil with a transformer and then through a series of capacitors and diodes of opposite polarity. This produces a V-high and a V-low. This must be designed to ensure that it is protected and does not exceed the breakdown voltage for the IC process being utilized.

This then powers up the switch circuitry. The output of this would send the switch command through a capacitor that couples into S2. As a result, every time the pacing pulse comes by, it would charge the system up. At the same time, a simple unit of logic would be performed that would send out a switch command onto S2, for example, at the tail end of the pace pulse.

An additional approach is to send a bipolar refresh command that goes high and low. The purpose of this approach is to provide back-biasing for transistors on the lead and to give them a higher conductance when they are turned on. In some cases, this circuitry would generate those signals as well.

### Fault Tolerant Operation

Current technologies for pacemakers have leads that are implanted into vessels and chambers of the human heart. When a failure occurs in one of these devices, removal of the device can be traumatic. After a long period of implantation, the leads adhere to the body, and there is a significant risk of trauma if the leads are surgically removed.

If a patient does not have access to medical care when part of the pacemaker device fails, a patient's health is at risk, until the patient can access medical care. Once a patient is in a surgical facility, the leads can be removed for replacement. However, removing implanted leads can cause trauma, as mentioned above. Also, the process of removing implanted leads typically requires significant time and expense.

Therefore, it would be desirable to provide a system that addresses a device failure in an implantable device and that does not require removing a portion of the implanted device.

The present invention provides systems and methods for addressing a failure in an implanted device without having to remove the implanted device. A processing device can detect a failure in an implanted device by sensing the performance of leads in the device. After a failure is detected, recovery or limited recovery can be accomplished by switching to a different lead, enabling different portions of the lead, or isolating failed portions of the lead. In some embodiments, functions of the device are retained after failure recovery is implemented. Redundancy is provided in an implanted device to allow failed portions of a device to be disabled without compromising functionality.

The present invention significantly reduces the risk of trauma to a patient, because the system does not need to be under a physician's direct care for it to detect and repair a failure. As a result, the patient's health is maintained, and the risk is reduced for the time it would take a patient to get to a medical facility to receive additional follow-up treatment. The present invention also provides a lower cost and less traumatic alternative to removing an implanted device.

Some types of recoverable pacemaker systems of the present invention have multiple implanted leads. According to some embodiments of the present invention, a controller, such as a pacemaker, can be coupled to leads with multiple electrodes. In some pacemakers, each of the electrodes is a programming electrode.

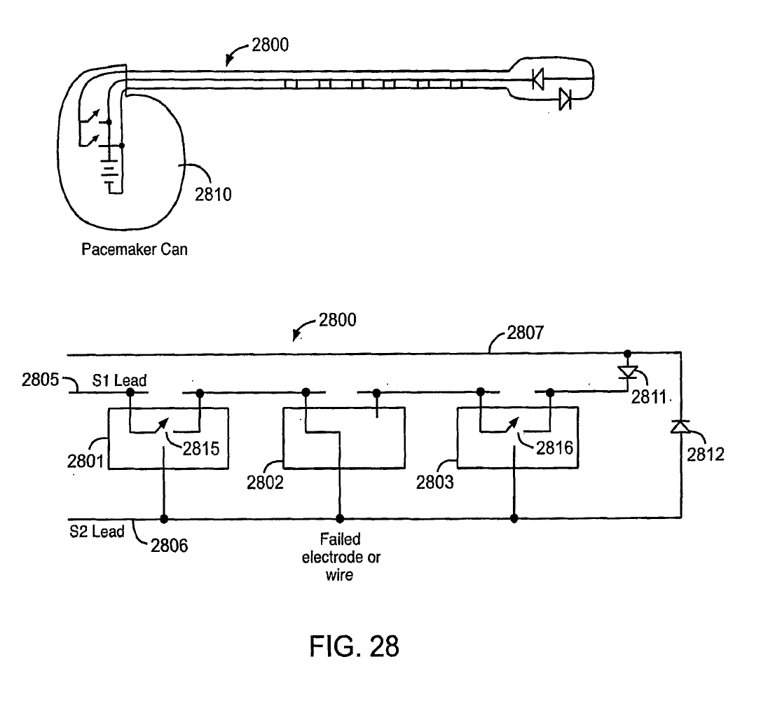

FIG. 28 shows a pacemaker can 2810 that is connected to an implantable device 2800 having three leads 2805-2807. Each of the leads comprises an electrical connection (e.g., a wire) between two or more elements. Leads 2805 and 2806 are coupled to multiple electrodes 2801-2803, as shown in an expanded portion of the diagram. The central electrode 2802 contains a fault. The fault in electrode 2802 is shown as a failed electrode with a short. Electrode 2801, proximal to can 2810, has a switch 2815. Electrode 2803 distal from can 2810, has a switch 2816. Switches 2815 and 2816 can be opened to isolate the shorted portion of electrode 2802.

Diode 2811 and diode 2812, in conjunction with auxiliary lead 2807, provide an additional path that can be used in the recovery of an open or shorted section, or of an isolated section (e.g., electrode 2802), as will be discussed in detail below.

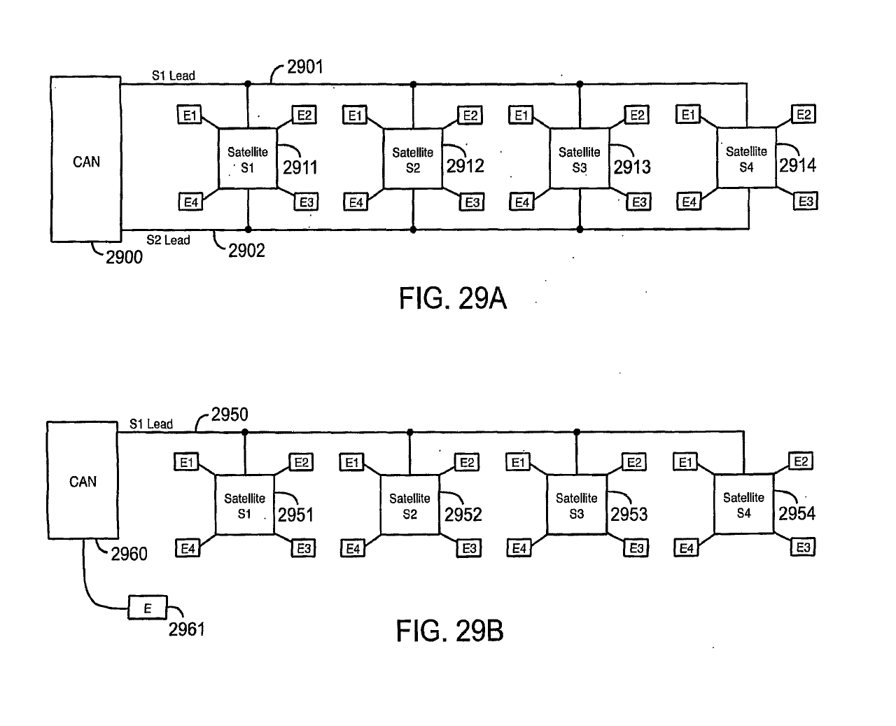

FIG. 29A shows an implantable medical device having two leads that are coupled to multiple satellites. Each of the satellites is coupled to four electrodes. A can 2900 at the left of FIG. 29A drives S1 lead 2901 and S2 lead 2902. Distributed along leads 2901-2902 are satellite S1 2911, satellite S2 2912, satellite S3 2913, and satellite S4 2914, each of which has four pacing electrodes, E1, E2, E3, and E4.

The normal flow of control and power is from can 2900 through lead 2901 to each of the satellites (e.g., satellite 2911-2914) and returning on lead 2902 to can 2900. According to an alternative implementation, a single lead is used in an implantable medical device, instead of the two leads shown in FIG. 29A. This implementation is shown in FIG. 29B. In FIG. 29B, a single lead 2950 carries power and control out to the various satellites 2951-2954. The return path is provided through the electrodes, E1, E2, E3, or E4, and through the conducting fluid of the body, either to can 2960 of the pacemaker or to an electrode 2961 provided as a return path.

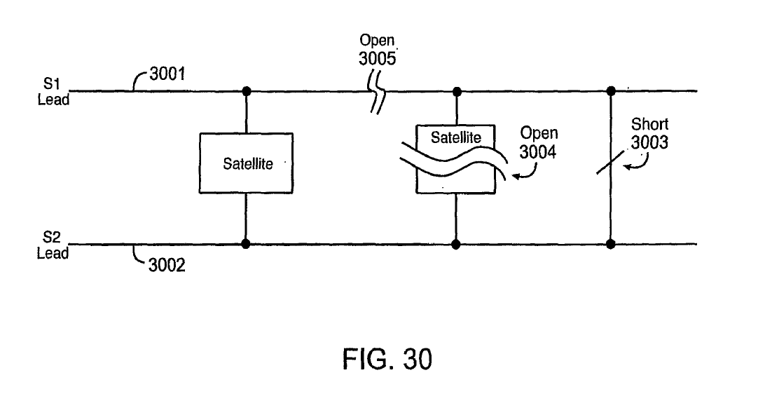

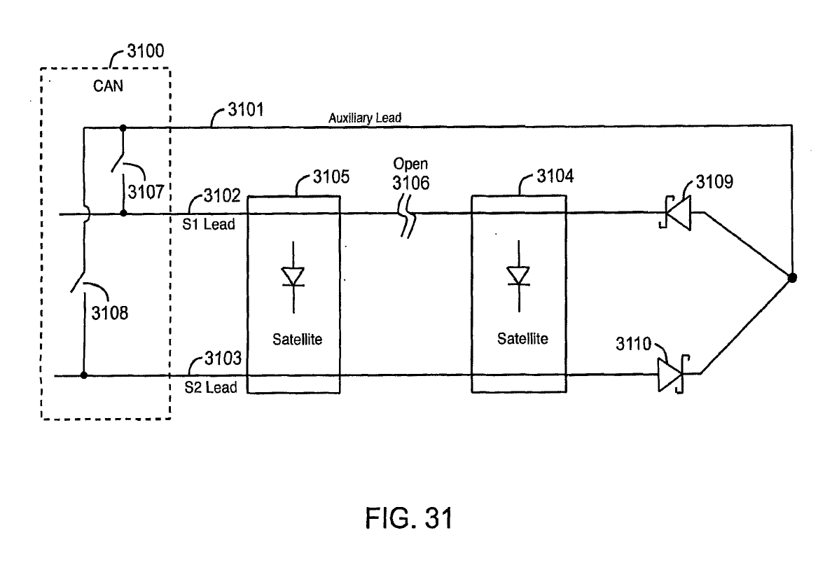

Numerous failures are possible on a lead system, such as the lead systems shown in FIGS. 29A-29B. FIG. 30 shows an open circuit 3005, where a lead 3001 has an open fault. FIG. 30 also shows an open circuit 3004 that has occurred in one of the satellites. Short 3003 is a direct short between leads 3001 and 3002. Short 3003 is a short circuit in one of the satellites in the device. Partial faults are also possible. As an example, on the way to becoming a full short, a lead may gradually fail, or conduction may gradually grow eventually creating a short. While the remainder of the present application refers to opens and shorts, the techniques of the present invention can also detect and address other types of faults in the same fashion.

An implantable medical device often includes an auxiliary wire. The auxiliary wire is a construction feature of some devices that is provided to add stiffness to the device. In prior art devices, the auxiliary wire has no function beyond providing stiffness and mechanical properties for the device. According to embodiments of the present invention, an auxiliary wire made of a conductive material can assist in providing fault recovery in an implantable device.

An example of an auxiliary wire that is used to provide fault recovery according to an embodiment of the present invention is shown in FIG. 31. FIG. 31 illustrates an implantable device with an auxiliary lead 3101. In FIG. 31, current flows from pacemaker can 3100, through S1 lead 3102, through a satellite (e.g., satellite 3104), and returning on the S2 lead 3103 to can 3100. FIG. 31 shows an open circuit that has been created in lead 3102 labeled open 3106.

Open 3106 effectively breaks the path for power and control that previously flowed through lead 3102, through satellite 3104, and returned on lead 3103. In FIG. 31, auxiliary lead 3101 is connected to lead 3102 by switch 3107 and to lead 3103 by switch 3108. Auxiliary lead 3101 is also connected to lead 3102 through diode 3109 and to lead 3103 through diode 3110 at the distal ends of these leads. Diodes 3109-3110 are oriented so that they conduct current for control or for power from auxiliary lead 3101, through diode 3109 and satellites 3104-3105, and returning on lead 3103.

Alternatively, diodes 3109-3110 allow power to return from lead 3103 through diode 3110, auxiliary lead 3101, and switch 3108 back to lead 3103 and can 3100. Diodes 3109-3110 prevent current from flowing in unintended paths. Diodes 3109-3110 are indicated as Schottky diodes for the lower voltage drop that is provided by a Schottky diode. The low voltage drop minimizes the effect on the system of using auxiliary path 3101 as a redundant mechanism.

Can 3100 contains a processing device (e.g., a controller) that is able to detect a failure, such as an open circuit, a short circuit, or an intermediate failure, on any of the leads or satellites in the implantable device. When can 3100 recognizes that it can no longer communicate with satellites (e.g., satellite 3104) down stream of open 3106, can 3100 sends a signal to switch 3107 that causes switch 3107 to close. Can 3100 then attempts to communicate with the satellites down stream from open 3106 again. After switch 3107 closes, communication between can 3100 and the satellites down stream from open 3106 is successfully restored by sending signals along auxiliary lead 3101 and through diode 3109. Thus, auxiliary lead 3101 provides redundancy to the device to bypass faults in the other leads or in satellites.

If an open circuit forms in lead 3103, closing switch 3107 does not restore communication with the satellites down stream from the open circuit. However, can 3100 can close switch 3108 to restore communications that are lost by an open circuit on lead 3103. After switch 3108 is closed, satellites that are down stream from the open circuit on lead 3103 can send signals to can 3100 along a path through diode 3110, auxiliary lead 3101, and switch 3108 back to lead 3103. The provision of switches 3107 and 3108, diodes 3109 and 3110, and auxiliary lead 3101 enable the implantable device to recover from a failure in either lead 3102 or 3103.

FIG. 31 demonstrates a mechanism for switching to an auxiliary lead 3101 to recover from a failure in leads 3102 and 3103.

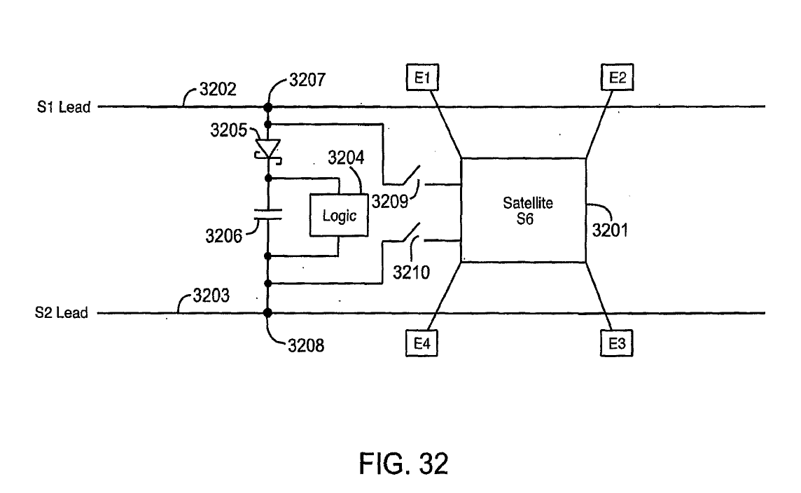

FIG. 32 illustrates a mechanism for isolating a portion of circuitry within one of the satellites that contains a failure, according to another embodiment of the present invention. In FIG. 32, satellite S6 3201 and its conducting electrodes E1-E4 represent an element of processing or function.

Satellite 3201 is shown as a pacing satellite with pacing electrodes in FIG. 32. However, satellite 3201 can alternatively perform another function. For example, satellite 3201 can perform a sensing function, such as a temperature sensor, a pressure transducer, or other type of measurement device. The use of a satellite in a pacing lead is shown and described herein for illustrative purposes only and is not intended to limit the scope of the present invention.

Additional circuitry is shown in FIG. 32 in conjunction with satellite 3201. The additional circuitry includes logic 3204, the connections of logic 3204 to lead 3202 and lead 3203 at junctions 3207 and 3208, and local power source or power storage devices, such as diode 3205 and capacitor 3206. The additional circuitry also includes switches 3209-3210.

Logic 3204 in FIG. 32 is powered when leads 3202 and 3203 provide power. Logic 3204 can communicate with a can (not shown) using any convenient protocol. Under the control of the can (or potentially under the control of logic 3204 acting autonomously), switches 3209 and 3210 can be opened, isolating satellite 3201 from the power provided through leads 3202 and 3203. Opening switches 3209 and 3210 enables the system to isolate a faulty satellite 3201, reducing the electrical load on the system.

Switches 3209 and 3210 also allow the system to potentially reduce power consumption by isolating functions performed by satellite 3201 that are not currently needed. A pressure transducer is an example of a function that is typically needed only on rare occasions. The switching mechanism shown in FIG. 32 allows a rarely used function to be enabled only at the times when its use is required. Logic 3204 enables the system under control of the can to either isolate faulted elements by opening switches (such as switches 3209-3210), or to reduce power by isolating those elements when they are not needed by opening switches.

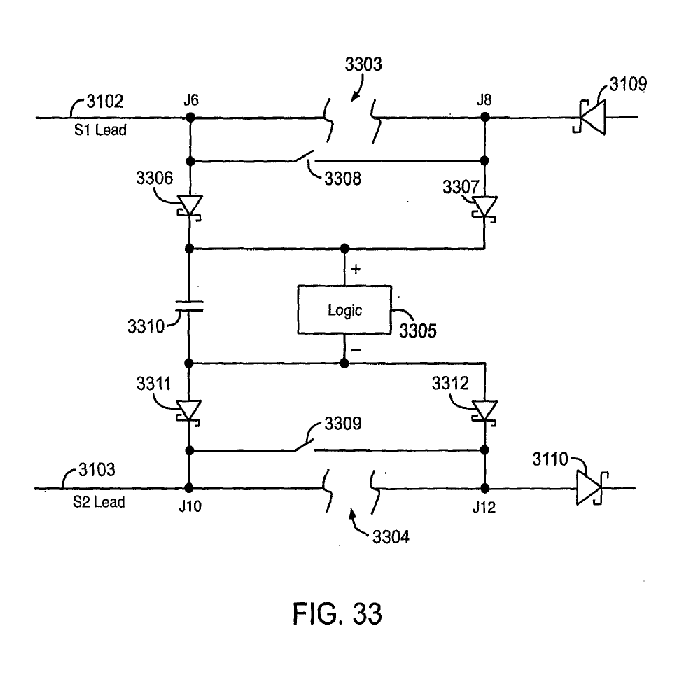

FIG. 32 illustrates a mechanism to isolate a function within one of the satellites. FIG. 33 illustrates an implantable device that can break electrical connections between two ends of an element to provide fault recovery, according to a further embodiment of the present invention. Breaking the electrical connections effectively isolates portions of a lead that are downstream from the break.

The system illustrated in FIG. 33 has utility in situations where there is a significant fault, such as a short circuit across two leads or an open circuit. Being able to open connections in the leads allows a fault to be isolated from other portions of the system. The embodiment of FIG. 33 includes switches 3308-3309 that are coupled to leads 3102 and 3103.

In FIG. 33, leads 3102 and 3103 have electrical breaks in them (e.g., that are caused by open circuit faults or created intentionally). The breaks are identified in FIG. 33 as break 3303 and break 3304. Logic element 3305 is connected to leads 3102 and 3103 on both sides of breaks 3303 and 3304. The connections between logic element 3305 and leads 3102-3103 are shown in FIG. 33 as junctions J6, J8, J10 and J12. Switches 3308 and 3309 are controlled by logic circuitry 3305. Switches 3308 and 3309 are indirectly under the control of the can, because the can controls logic circuitry 3305.

Logic 3305 can close switch 3308 to provide an electrical connection between junctions J6 and J8 that bypasses break 3303. Logic 3305 can close switch 3309 to provide an electrical connection between junctions J10 and J12 that bypasses break 3304. Thus, switches 3308 and 3309 can be closed to conduct across breaks 3303 and 3304, respectively.

Power for logic element 3305 can be obtained from either the proximal or distal ends of lead 3102 or the proximal or distal end of lead 3103. Diodes 3306 and 3307 can conduct power from junction J6 and junction J8, either of which may provide power to capacitor 3310 and logic 3305. Diodes 3311 and 3312 conduct power or provide a return path for power from capacitor 3310 and logic 3305 at junctions J10 and J12 on lead 3103.

Capacitor 3310 provides stored energy for logic element 3305. Diodes 3306 and 3307 allow power to reach logic element 3305 from either the distal end or the proximal end of lead 3102. When switch 3308 is open, and a break exists in lead 3102 at break 3303, power does not flow between functions J6 and J8.

If power is not being provided from the can to junction J6 because of an open circuit in the proximal end of lead 3102 (to the left of J6 on the figure), power can be routed to logic 3305 through along a current path through auxiliary lead 3101 shown in FIG. 31, Schottky diode 3109, junction J8, diode 3307, and eventually to capacitor 3310. If there is a break in lead 3103 that is proximal to junction J10 (left of J10 on the figure), a return path can be provided from capacitor 3310 through diode 3312, junction J12, Schottky diode 3110, and auxiliary lead 3101. The auxiliary lead 3101 and diodes 3109 and 3110 provide redundant paths that allow logic 3305 to function despite the existence of a fault in either lead 3102, or lead 3103. Lead 3103 is addressed by auxiliary lead 3101 through the second diode 3110 at the distal end of lead 3103.

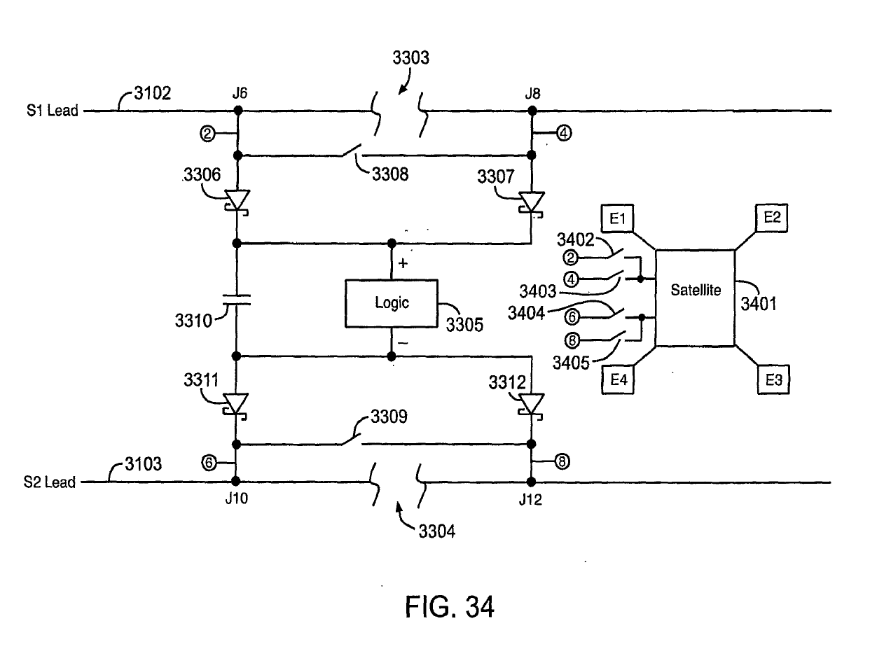

The previous figures in the present application have shown isolated functions for powering satellites along a lead and bypassing a fault that has occurred in a lead. FIG. 34 shows a combination of these two functions that are performed in a single section of an implantable device. In the example of FIG. 34, leads 3102-3103 have breaks 3303 and 3304. Junctions J6 and J8 are between break 3303 in lead 3102, and junctions J10 and J12 are between break 3304 in lead 3103. An additional element, satellite 3401, receives power from either the distal or the proximal end of lead 3102 through switches 3402 and 3403. Switch 3402 is coupled to junction J6 at connection point 2, and switch 3403 is coupled to junction J8 at connection point 4. Power return is provided along the distal or the proximal end of lead 3103 through switches 3404 and 3405. Switch 3404 is coupled to junction J10 at connection point 6, and switch 3405 is coupled to junction J12 at connection point 8. Switches 3402-3405 are under the local control of logic 3305 and the indirect control of a pacing can or other processing device.

While the function of logic 3305 and satellite 3401 are shown as distinct blocks in FIG. 34, they may be implemented in a variety of fashions. They may be integrated in a single piece of semiconductor, e.g., as a single integrated circuit. Alternatively, the functions performed by logic 3305 and satellite, 3401 can be integrated into separate chips and individually mounted within pockets in a can or mounted at a location along a lead. A wide variety of other implementations are also possible.

Creating breaks (such as breaks 3303 and 3304) in leads 3102 and 3103 has the potential to create weaknesses in the mechanical design of a system. If a lead is broken and then bridged by one of the bypass mechanisms of the present invention, the mechanical forces that are normally transmitted through the lead are instead, transferred to the can and to the satellite that performs the function at the break point.

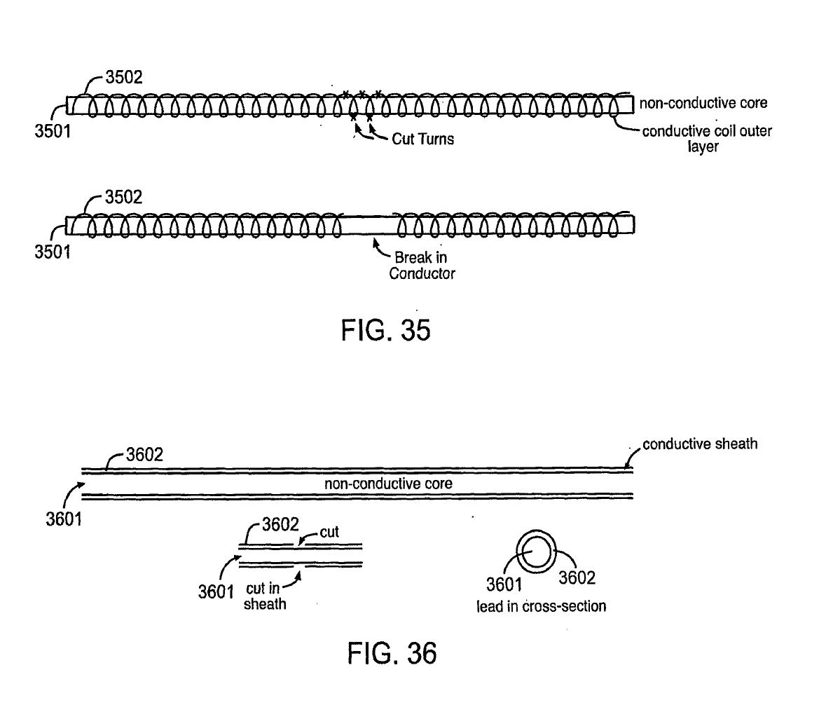

FIGS. 35 and 36 show two embodiments for creating a break in conductive leads (such as breaks 3303 and 3304), according to further embodiments of the present invention. FIG. 35 shows a lead with a nonconductive core 3501 that provides strength, and a coiled wire 3502 wrapped around core 3501. Cutting several small sections of wire 3502 creates an opening/electrical break in that wire. After the cuts have been made in wire 3502, the internal core 3501 remains intact and continues to provide strength to the lead, while the electrical conductivity at the cuts in wire, 3502 is broken. The cuts in wire 3502 are examples of how breaks 3303 and 3304 can be formed. Connections to wire 3502 can be made on either side of the cuts. For example, the connections can be made at the junctions shown in the previous figures.

FIG. 36 shows an alternative means for creating an electrical break in a conductive lead that allows the lead to maintain its physical strength. In the coaxial conductor shown in FIG. 36, the center strength of the conductor is provided by a nonconductive core 3601. Core 3601 is surrounded by a conductive sheath 3602. The conductive sheath 3602 can be cut to form an electrical break in the conductor, while core 3601 remains intact.

A tool analogous to a pipe cutter used in home construction can be used to create a cut in sheath 3602. The tool can be clamped down around the combined coaxial core 3601 and sheath 3602. A cutting element can clamp down onto conducting sheath 3602. As the tool is rotated around conductive sheath 3602, it creates a cut in conducting sheath 3602. The tool is adjustable so that the cut can fully sever the electrical connection without damaging internal core 3601.

The previous discussion has dealt with the mechanical and electrical capabilities of the switching elements and satellites, as well as with mechanisms that can be used to bridge or open conductive paths. A recovery strategy for faults is now described in detail. When a system is initially powered up, it is not known if the system contains faults. According to further embodiments of the present invention, a mechanism is used to incrementally add power to portions of a device. If the device is working properly, there will be no interruption to the power-up sequence. One element after another in the device is powered up, until all elements in the device have power, and the device is functioning fully. As power is progressively applied to ever more distal sections of the device, eventually a fault may be uncovered, as detected by an increase in current or an inability to communicate with another element of the device.

The present invention includes a basic mechanism for both power-up and fault recovery. During power-up, elements in a device are sequentially powered. Eventually, the device is fully operational. This same process is followed after a fault. When a fault is detected, the last switch that has been closed is the last switch before the fault, and that last switch can be opened to isolate the fault, according to the present invention.

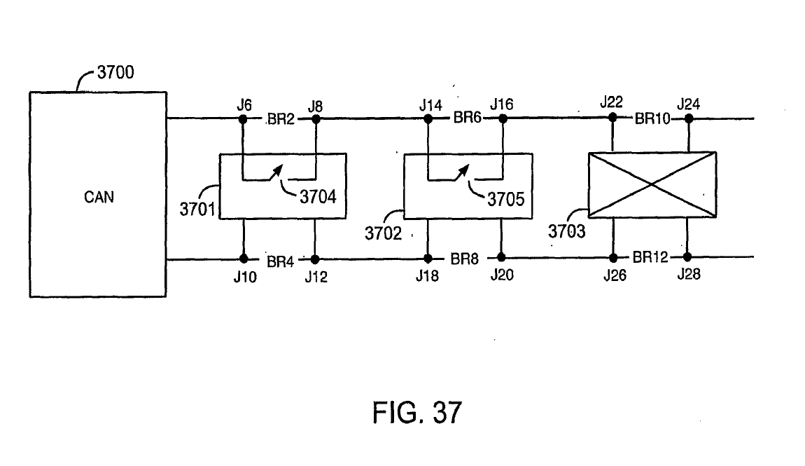

FIG. 37 illustrates how the flow of power can be incrementally applied proximal to the distal end of a lead to detect a fault, according to an embodiment of the present invention. In FIG. 37, can 3700 contains a processor that applies a power-up sequence to satellites 3701-3703. Satellite 3703 contains a fault. Switch 3704 in satellite 3701 is initially be closed, bridging from junctions J6 to J8, and allowing power to reach switch 3705. When a switch 3705 in satellite 3702 is open, can 3700 does not detect a fault, and the system continues to function properly. Closing switch 3705 allows power to reach failed satellite 3703. At this point, the error in satellite 3703 causes a significant and unexpected increase in current consumption. When can 3700 detects the increase in current consumption, it causes switch 3705 to open in order to isolate the fault at satellite 3703, allowing satellites 3701-3702 to operate normally.

In a similar fashion, once a fault has been detected, or a portion of the lead has been isolated, an auxiliary lead, as shown in FIG. 31, can be used to access isolated satellites. The device has a path through the auxiliary lead to reach satellites and other elements on the far distal end of the lead. When power is initially applied to the lead starting at the can, the system can apply power through the auxiliary lead from the distal end of the main lead moving toward the proximal end of the main lead, until the can uncovers a fault. The sensing mechanism using the auxiliary lead and the ability to add one satellite at a time is analogous to the process previously described that moves from the can outward through the main lead.

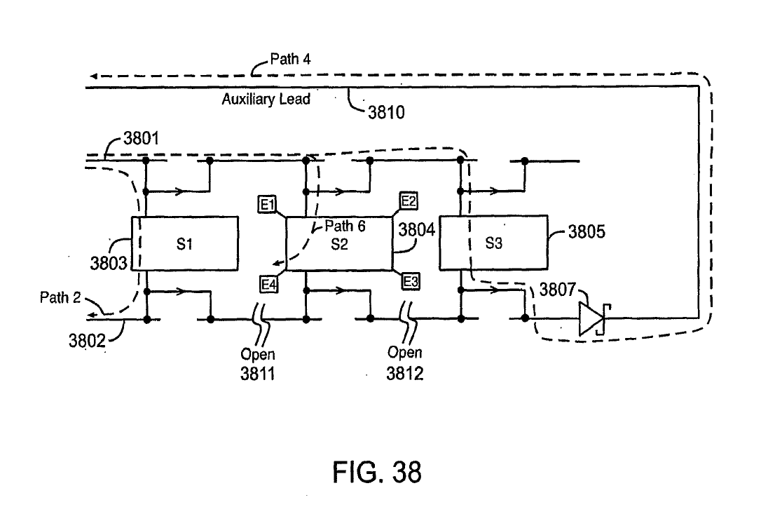

FIG. 38 illustrates another technique for recovering from a fault, according to a further embodiment of the present invention. The above discussion covered paths 2 and path 4, shown in FIG. 38. Path 6 is an additional recovery mechanism. In path 2, control flows from lead 3801 through satellite 3803 and returns on lead 3802 following path 2. As discussed above with respect to the previous figures, an additional recovery path 4 uses the auxiliary lead 3810. The recovery path 4 flows from lead 3801 through satellite 3805, through diode 3807, and returning on auxiliary lead 3810.

A third path 6 shown in FIG. 38 enables satellite 3804 to function even though open circuits 3811 and 3812 exists on lead 3802, both proximal and distal to satellite 3804. In this failure mechanism, no return paths exist along either lead 3802 or auxiliary lead 3810. In this situation, satellite 3804 can revert to one-wire operation using just lead 3801, while the remainder of the system, including satellites 3803 and 3805 continue to operate in a normal two-wire mode.

### Overvoltage and Overcurrent Protection

In the case of cardiac devices, overcurrent protection is an important component in the circuit to avoid inadvertent tissue damage by injury current to a patient in the case of internal or external high voltage shock which may be required for defibrillation therapy. Protection from overvoltage effects would also useful in maintaining the advantages of the device. However, presently available overcurrent and/or overvoltage protection strategies are not applicable to highly miniaturized medical devices.

Efforts have been made to provided for overvoltage protection in highly miniaturized devices using discrete high voltage MOS devices Transient Voltage Suppressor, Zener Diodes. However, this approach requires that the protection circuitry resides outside of the chip, increasing production difficulties and uncertainties, and requiring a considerably larger size for the device.

The present invention provides strategies for circuitry configurations that provide both overcurrent protection in the circuit to avoid inadvertent tissue damage and overvoltage protection of circuitry in highly miniaturized medical devices to avoid tissue damage to patients from injury current. The invention is particularly suitable when there are satellites connected in a chain to S1 and S2 wires, such as those previously described by some of the present inventors, described below.

The present invention provides protection circuit strategies that provide protection against over-current to protect tissue and over-voltage to protect circuitry in highly miniaturized medical devices. The present inventive protection circuit is particularly suited to the new and novel concept of multiplexing pacing and sensing signals developed by some of the present inventors. New configurations of the implantable circuitry are necessary to meet strict miniaturization requirements while limiting or eliminating injury current damage to intervening tissue between electrodes connected to circuitry and overvoltage compromise or destruction of device circuitry.

Potential injury current challenges are presented by defibrillation and other high-voltage therapy needed to accomplish critical clinical goals, such as defibrillation. When injury current reaches tissue which the device is contacting, especially at focused contact points such as an electrode, a resulting large electrical and resulting heat surge will occur similar to that used clinically for ablation proposes. However, in the case of inadvertent healthy tissue “ablation” style damage from injury current, serious tissue damage and destruction can occur. This is not good clinical practice, especially in a cardiac challenged patient, whose health could be further compromised by the procedure.

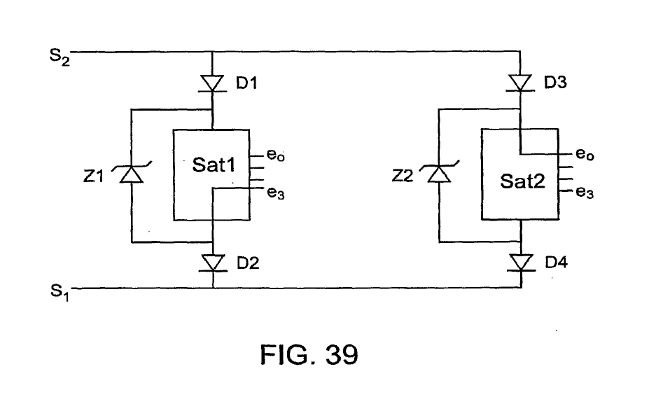

As shown in FIG. 39, the inventive protection circuitry is described in the embodiment when there are satellites connected in a chain to S1 and S2 wires. Satellite 1 and satellite 2 are provided. Each satellite is connected to wires S2 and S1 through two diodes.

Satellite 1 is connected through diodes D1 and D2. Likewise, satellite 2 is connected through diodes D3 and D4. Across each satellite inside the diodes that connect the satellites to the wires S1 and S2, there is a Zener diode. Satellite 1 has Zener diode Z1 across. Zener diode Z1 is inside diodes D1 and D2. Satellite 2 has Zener diode Z2 across it. Zener diode Z2 is between satellites and diodes D3 and D4.

Each of satellites 1 and 2 has four electrodes, e₁, e₂, e₃, and e₄ coming out of them. These electrodes can be configured internally to be either connected to S1 or to S2.

The inventive protection scheme assures that if there is a higher voltage on any of the electrodes e₁, e₂, e₃, or e₄, on one satellite as compared to that on the electrodes of a second satellite, current is prevented from traveling from electrodes on one satellite to the electrodes on the other satellite, and injury current avoided while the circuitry on each satellite is protected from over voltage. By example, in the event of a high voltage event on electrode 0 on satellite 2, current flowing from electrode 0 on satellite 2 to some electrode on another satellite must be protected against. The diodes D1, D2, D3, and D4, and the Zener diodes Z1 and Z2 as configured in the embodiment of present invention here shown provide such protection. The current between satellites is squelched while voltage imbalances across a satellite are limited within a safe range.

By example, when on satellite 1, electrode 3 is connected to wire S1, and electrode 3 experiences a high voltage event compared to satellite 2. This event will forward bias diode D2. As a result, the current will flow through diode D2 and get to diode D4, which is reverse biased. There, the current is clamped to a leakage level. In this manner, injury current is stopped from flowing through E3 if there's a high voltage on satellite 1 relative to satellite 2. In another example, on satellite 2 electrode 0 is connected to S2. In the case of a high voltage on satellite 2 relative to satellite 1, the current would to flow through E0 and get to diode D3. Because diode D3 is reverse biased, only leakage current will follow through diode D3. Continuing, the high voltage could cause current to flow through the Zener diode Z2 and flow through Z2 and then come to D4. The current will flow through D4 and then get to D2. Because D2 is reverse biased, the current will not flow, the circuit will not be closed, and therefore potential damage from excessive current is avoided.

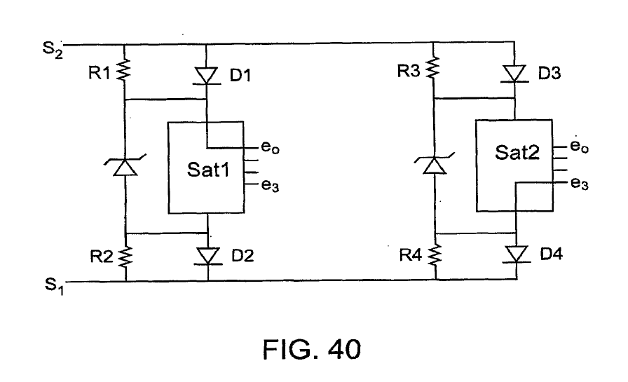

As shown, in FIG. 40, similar to FIG. 39 above, the inventive overvoltage protection circuitry is described in the embodiment when there are satellites connected in a chain to S1 and S2 wires. FIG. 40 provides a schematic view of an embodiment providing a sensing capacity.

Satellite 1 and satellite 2 are provided. Each satellite is connected to wires S2 and S1 through two diodes. In this case, the addition of resistors R1, R2, R3, and R4 provide for additional sensing capacity over the embodiment show in FIG. 39. Resistors R1, R2, R3, and R4 can be from about 20-100 k, preferably about 30-70 k, and most preferably about 50 k.

The objective of the device incorporating the protection circuits of the present invent is intended for high-voltage protection circuitry for implantable small integrated circuitry, e.g., as described above. The circuitry first senses a high voltage event at a particular electrode. It responds by preventing that voltage from flowing to the other electrodes, thereby generating injury current from the overcurrent.

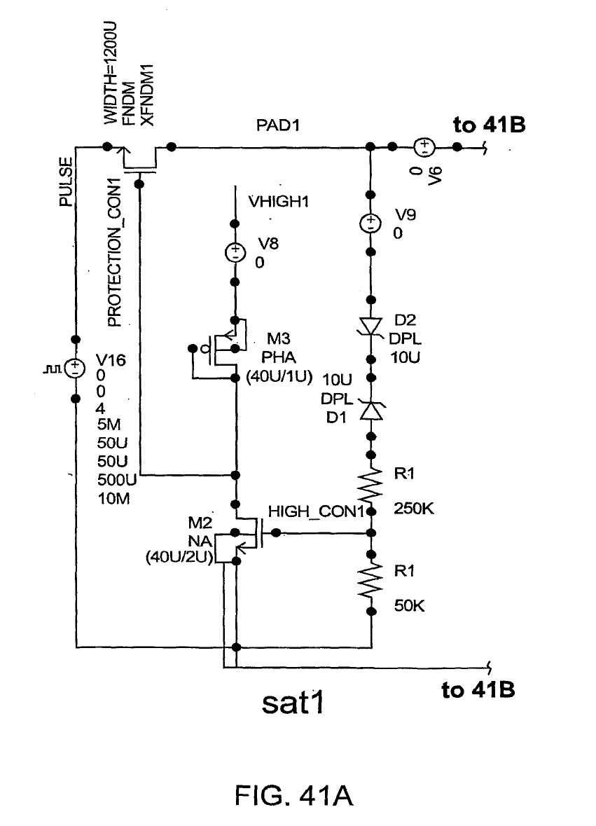

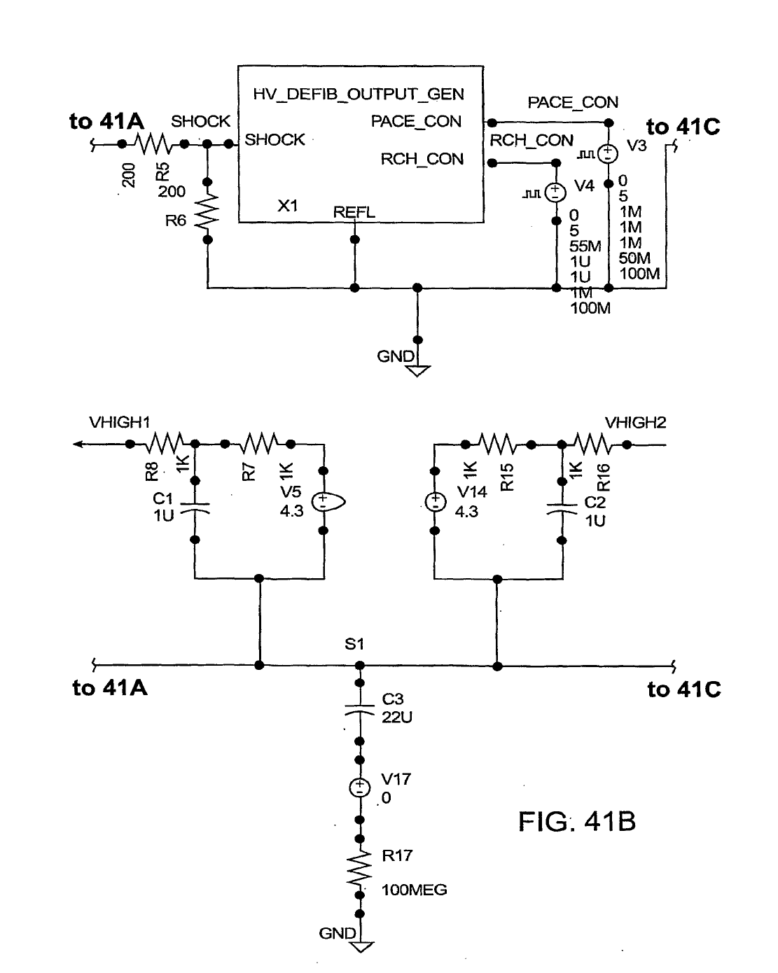

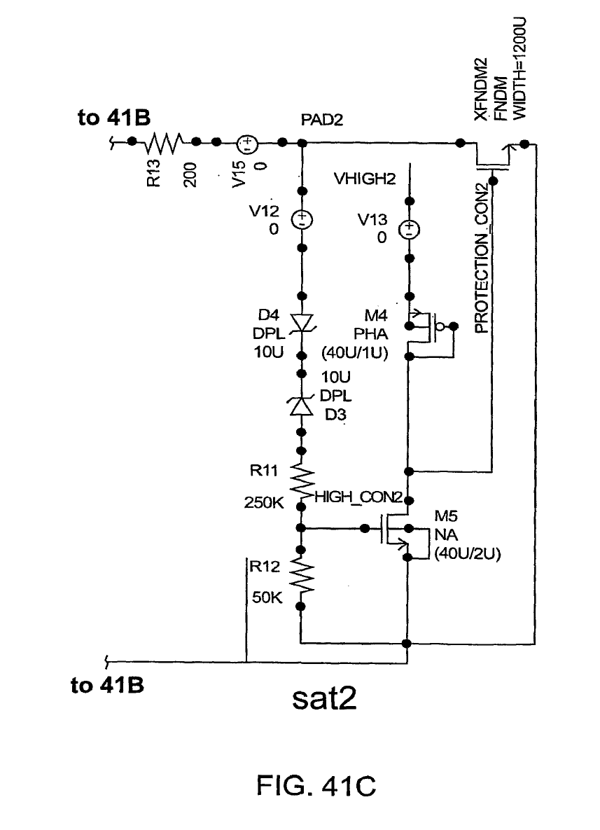

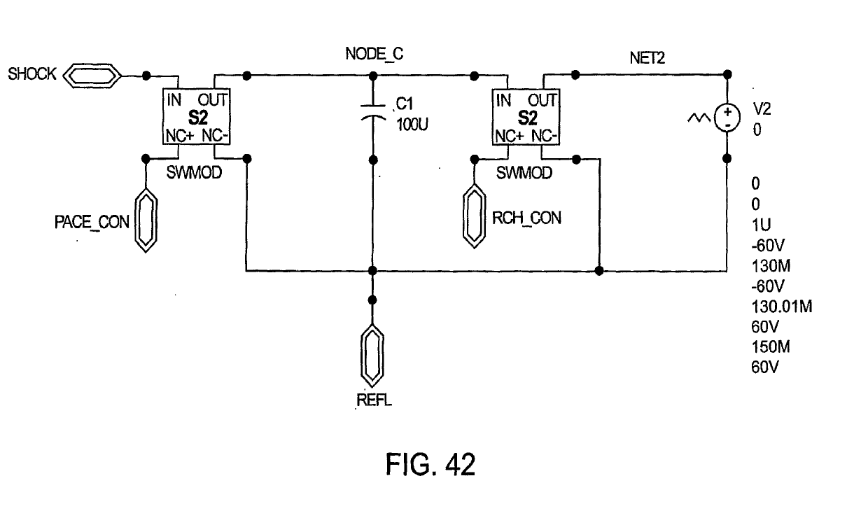

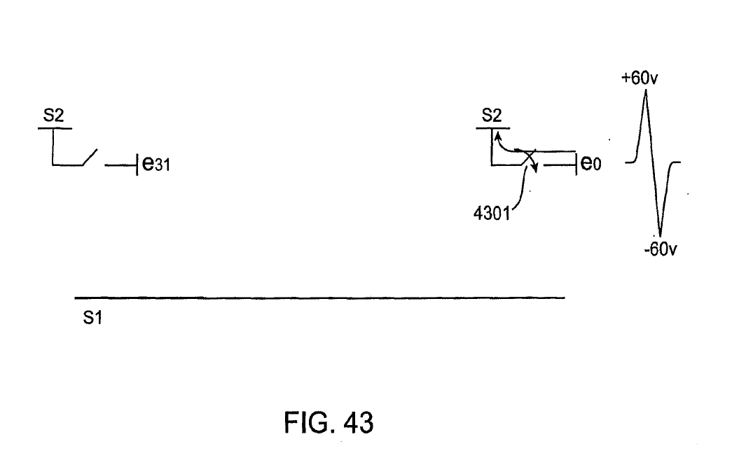

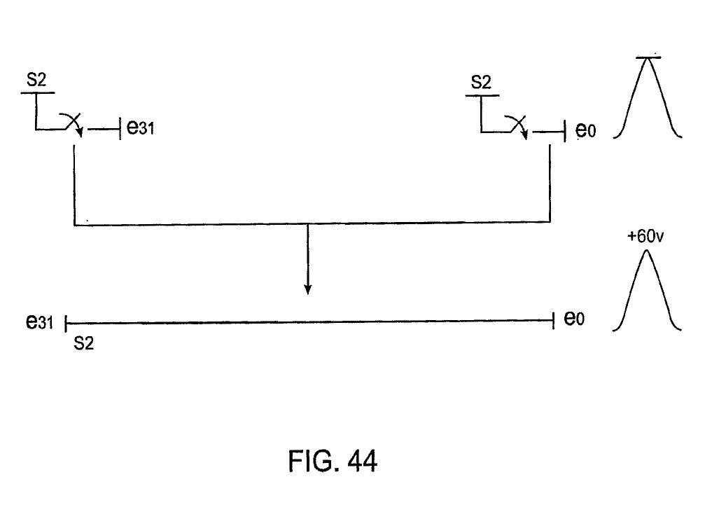

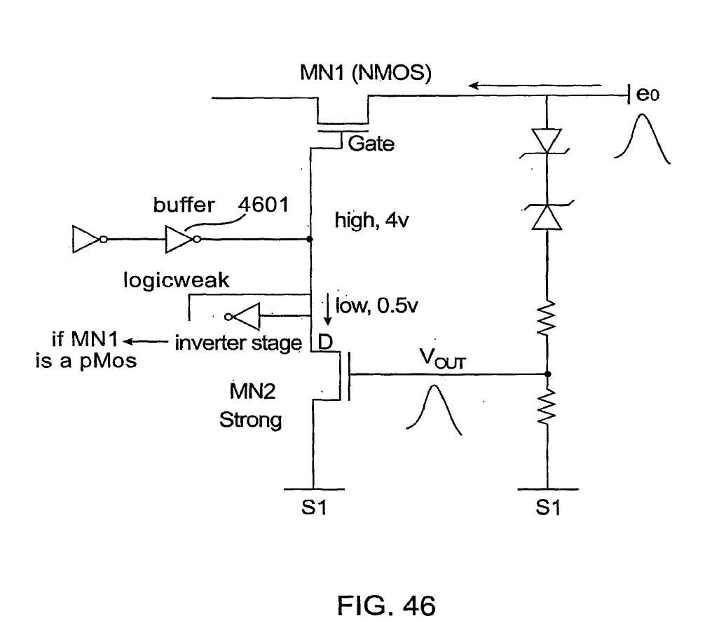

FIGS. 41A-C are schematic representations of the example shown in FIG. 46. FIG. 42 is a representation of the defibrillation module as seen in FIGS. 41A to 41C. FIGS. 43-47 provide diagrams showing the mechanism by which this objective is accomplished. Referring now to FIG. 43, a given electrode e0, is connected through switch 4301 to a pacing or sensing line S2, for pacing. Electrode e0 is paced with line S2 with respect to line S1. In this configuration, switch 4301 is closed to connect electrode e0 to S2. This will put a charge into S2 which will discharge through electrode e0 and switch 4301 into line S1. As shown in FIG. 44, if, within this system, there is a high voltage event on electrode e0, for instance a defibrillation shock, putting the surge at about +/−60V, that current will flow towards low-voltage electrode e31. This event is potentially damaging for any tissue in contact with either involved electrode

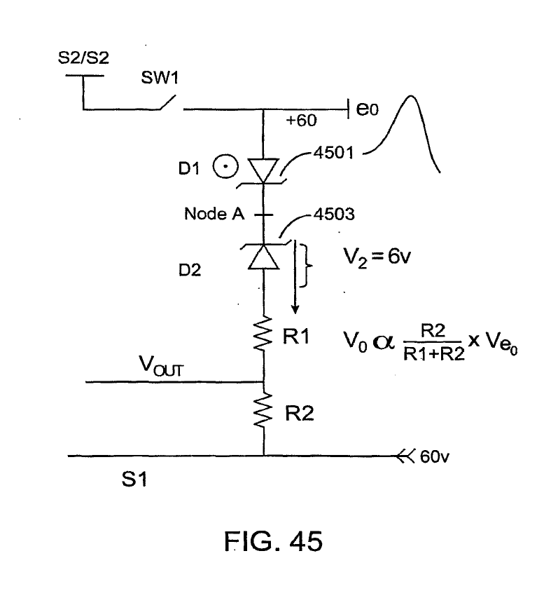

FIG. 45 provides a representation of how to detect the occurrence of a high voltage event there. A high voltage event detector is implemented with Zener diodes. This detector is two Zener diodes 4501 and 4503 connected to each other in a back to back configuration. This is sent that through a resistor-voltage divider, and then that goes to the reference, which is in this case S1. By example, consider the occurrence of, a high-voltage, such as a sixty volt surge. S1 is at lower voltage, which is considerably lower than sixty volts. As a result, the current would flow towards S1. However, the intervening Zener diodes of the present invention are provided with a breakdown voltage.

The first Zener diode 4501 will forward bias. This result is because Zener diode 4501 is connected in forward bias configuration, and it will start conducting: Next, Zener diode 4503 will not conduct until the voltage on node A reaches its breakdown limit.

By example, the breakdown limit of this Zener diode is six volts so when this Zener diode sees six volts across it, it breaks down. As a result, it permits the current to flow through it. The current, flows through, and goes through two resistors R1 and R2. These two resistors act as a resistive voltage divider. R1 is set as a ratio with respect to R2. Voltage V0 is proportional to the ratio of R2 divided by R1 plus R2 multiplied by the voltage we see at the electrodes. In this way, a large voltage is scaled to a smaller voltage.

At the same time, the inventive circuitry functions to detect whether there is a low or high voltage event. In the absence of a high voltage event, which is really detected by this Zener diode, for example if a pulse is at ten volts, this diode would not break down. As a result, that voltage level is detected as a normal voltage event. A pacing pulse would be a typical example of such a voltage level. A high voltage detection would not come into effect.

As shown in FIG. 46, in the presence of high voltage event, the inventive circuitry will detect this state. This detection is used to control switch MN1. The high voltage event detection is accomplished with two Zener diodes and two resistors. Shown is an electrode and switch, which can be a CMOS switch. The electrical flow runs through a transistor which is a CMOS device. The flow is processed through another switch, which again can be an CMOS switch. As a result, when a high voltage event occurs on electrode e0, Vout will go high. This will turn on MN2. When MN2 gets turned on it drives the gate of MN1 through the potential S1. Essentially, it turns off MN1, which prevents current flowing from e0 further from anywhere else. Electrode e0 is isolated from the circuitry by an open circuit.

The purpose of this embodiment of the inventive circuitry is be able to control the state of that switch MN1 in normal operating mode. That is, if there is no high voltage event on e0, the logic circuitry will determine the state of MN1. By example, the circuitry described above will be able to control the state of that switch, to be on or off. Only in the event that a high voltage event occurs will MN2 be affected.

MN2 will turn the switch off regardless of what the logic wants the switch to be at. To accomplish that, the logic signal is hooked up through a buffer. In this case, buffer 4601 is hooked up to the gate of MN1. The device is configured so that MN2 is made to be strong. Buffer 4601 is configured to be weak. As a result, in this example when buffer 4601 attempts to turn the gate on, it will force the gate for example to logic high. This can be in the range of four volts. When a high voltage event is detected and switch MN2 turns on, switch MN2 will force the gate to go to logic low, as an example at about 0.5 volts.

Since MN2 is much stronger than the weak buffer 4601, when it turns on it will force the output state of buffer one to level low as well. When the event goes away and MN2 turns off the buffer, the gate can be turned on again.

The above describes a system which shows the basic principle behind this operation but many others are available within the teachings of the present invention. The same configuration can be used if the MN1 is off an PMOS switch. If MN1 becomes a PMOS switch, another inverter stage is added at the drain on MN2. This then goes through that inverter stage and drives the gate.

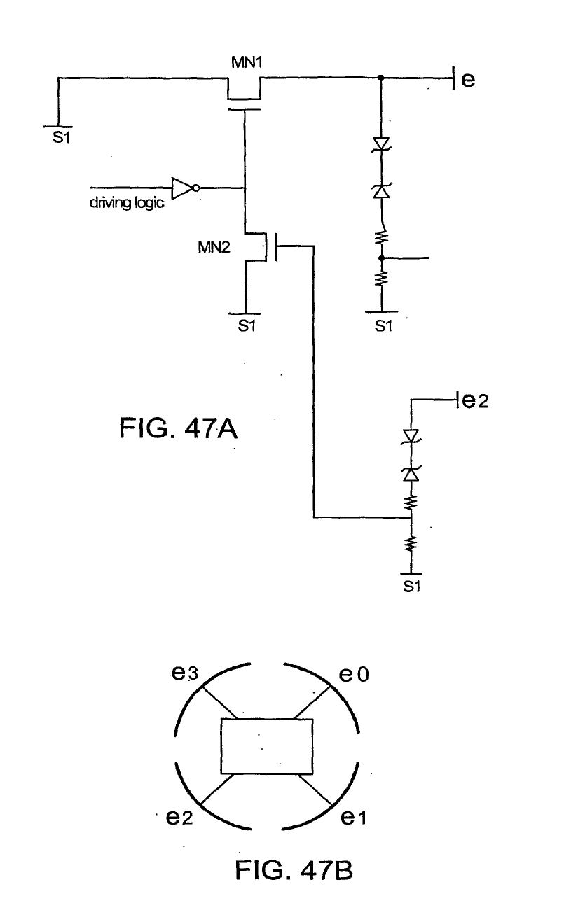

FIG. 47A provides a specific example of the above configuration in a real application employing an integrated circuit of the invention, as described above. In this example, an integrated circuit chip is provided with four electrodes on a given satellite, shown as e0, e1, e2, and e3. The detection circuit in this example is used if switch MN1 is initially in the on mode. This would occur when electrode e0 is connected to line S1. In effect, a short circuit is created across the detection circuitry. One side of the detection circuit is connected to S1, the bottom side of the resistor. The side that is connected to electrode e0 through switch MN1 is also connected to S1.

In the case of a voltage event both sides of this detection circuitry, they are at the same potential, and will not detect a voltage event. To avoid this effect, consider the multi-electrode lead consisting of e0, e1, e2, and e3. FIG. 47B shows e0 and e3, which are in contact with the heart tissue. Electrodes e1 and e2 are not in contact with the heart tissue. When using e0, its opposing electrode, which is e2 must be off. To provide for that, e2 is provided a switch MN3 (not illustrated). MN3 has a switch that goes to S1. It must be assured that this switch is in the off mode.

Electrode e2 also has a detection circuitry attached to it, providing detection electrode e2. Detection circuit 2 is different than detection circuit 3 of e0, as both of its ends are not shorted to the same potential as detection circuit 1. Control e0 can act as a detection circuitry. As a result, even though there is a short, e2 is not be effected by it. Because e2 is opposing the heart tissue and is not in contact with it, it can be turned off and used as a high voltage detector.

The same approach can be applied to e3 versus e1. It is possible to determine where each one is connected, and make the opposing ones. This approach can be expanded this to any configuration. For instance, the adjacent rather than the opposing can be used, or an electrode that it is not is used for detection. This provides a fail-safe approach without interrupting service.

As described above in conjunction with FIG. 39, diodes can be used to prevent the satellites and the two bus wires from forming a low impedance circuit during defibrillation, which can result in a high current density around an electrode and injure the tissue. A diode, however, introduces an additional 0.7 V voltage drop when forward biased, which reduces the efficiency of the power-supply to the satellites. One embodiment of the present invention employs a transistor based current limiting circuitry to reduce this additional voltage.

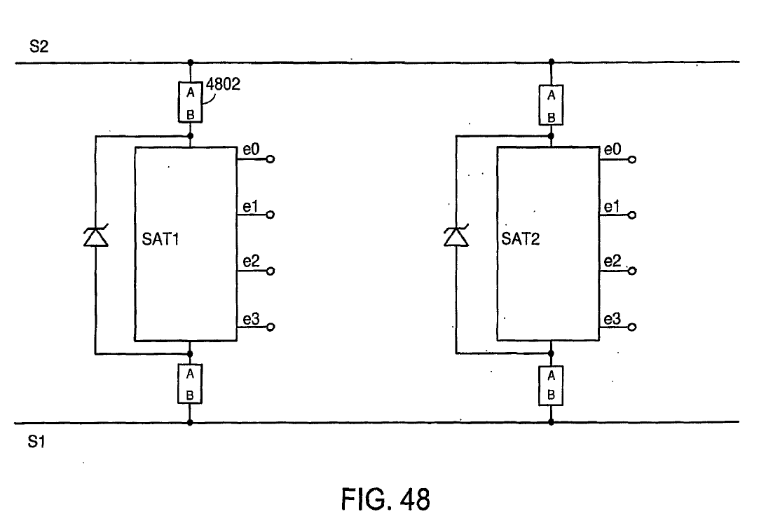

FIG. 48 is a block diagram illustrating a configuration that uses transistor-based current limiting circuitry to protect the satellites and tissue from over current, in accordance with one embodiment. Instead of using diodes, this configuration uses transistor-based current limiting circuitry, such as circuitry 4802, to prevent excessive current from flowing from the satellites to the S2 wire, which is presumed to provide a high-voltage power supply, and to prevent excessive current from flowing from the S1 wire to the satellites.

Current limiting circuitry 4802 has two ports, A and B. According to one embodiment, current limiting circuitry 4802 allows current to flow from port A to port B with a minimal voltage drop, but prevents excessive current flowing from port B to port A, thereby protecting the satellites from over current and also preventing high current density from forming at any electrodes.

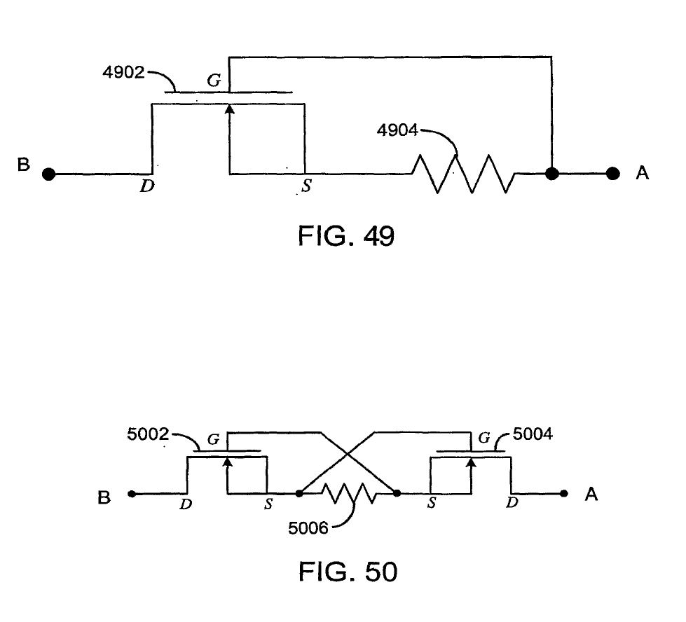

FIG. 49 is a schematic circuit diagram illustrating a uni-directional current limiting circuitry, in accordance with one embodiment. This circuitry includes a depletion-type NMOS transistor 4902 and a resistor 4904. The source of NMOS transistor 4902 is coupled to one side of resistor 4904, and the gate of NMOS transistor 4902 is coupled to the other side of resistor 4904. When a current flows from port A to port B, resistor 4904 introduces a voltage drop between the gate and source of NMOS transistor 4902. This positive V-Gs allows the depletion-type NMOS transistor 4902 to stay on and allows the current to flow from source to drain.

When a current is flowing from port B to port A, the voltage drop over resistor 4904 can create a negative gate-to-source voltage. That is, V_{GS}<0. This negative gate-to-source voltage, when sufficient, can pinch off the conduction channel in the depletion-type NMOS transistor 4902 and prevent additional current from flowing from port B to port A. The pinch-off threshold current is determined based on the design of NMOS transistor 4902 and the resistance of resistor 4904. For example, a properly set of chosen parameters can produce a threshold reverse current of about 50 mA.

The current limiting circuitry shown in FIG. 49 provides protection against over current in one direction. However, when multiple satellites are coupled to the two bus wires, the cumulative reverse current from many satellites could still result in a large current flowing through one satellite in the allowable direction. For example, referring to FIG. 48, assume there are eight satellites coupled between S2 and S1, and that each satellite allows 50 mA of current flowing back to S2. One satellite could become the low-impedance passage through which the aggregation of these currents, which can be as high as about 50×8=400 mA, passes. According to one embodiment, such an aggregate current is referred to as a “gang current.”

One embodiment of the present invention employs a bi-directional current limiting circuitry to prevent formation of a gang current. FIG. 50 is a schematic circuit diagram illustrating a bi-directional current limiting circuitry, in accordance with one embodiment. The circuitry includes two depletion-type NMOS transistors, 5002 and 5004. The sources of NMOS transistors 5002 and 5004 are coupled to the two sides of a resistor 5006. The gate of NMOS transistor 5002 is coupled to the source of NMOS transistor 5004, and the gate of NMOS transistor 5004 is coupled to the source of NMOS transistor 5002.

When a small current is flowing from port A to port B, resistor 5006 causes the gate-to-source voltage on NMOS transistor 5004 to be negative. If the current is sufficiently small, the conducting channel on NMOS transistor 5004 remains on to allow the small current to pass through. Meanwhile, resistor 5006 also causes the gate-to-source voltage on NMOS transistor 5002 to be positive, which ensures that NMOS transistor 5002 is on. When the current from port A to port B surpasses a threshold, resistor 5006 causes the gate-to-source voltage to drop below the pinch-off voltage and turns off NMOS transistor 5004, thus preventing excessive current from flowing from port A to port B.

Similarly, when a current flowing from port B to port A is sufficiently small, NMOS transistor 5002's gate-to-source voltage is above the pinch-off threshold and the conduction channel of NMOS transistor 5002 remains turned on. NMOS transistor 5004 is also turned on because its gate-to-source voltage is positive. When the current from port B to port A surpasses the pinch-off threshold, NMOS transistor 5002 is pinched off, preventing additional current from flowing port B to port A.

According to one embodiment, for a desired current limiting value I_{D}, the resistance of resistor 4904, R₁, can be chosen based on the following formula:

$${R}_{1} = \frac{{V}_{GS ( OFF )}}{{I}_{D}} \cdot ( \sqrt{\frac{{I}_{D}}{{I}_{DSS}}} - 1 )$$

Where I_{D} is the desired current limiting value, V_{GS(OFF)} is the pinch-off voltage for the depletion-type NMOS transistor, and I_{DSS} is the saturation current at V_{GS}=0V. Note that both V_{GS} and I_{DSS} are device dependent parameters. In one embodiment, the current limiting circuitry is configured to limit the maximum current to 50 mA with an error margin of +0% and −5%. In a further embodiment, the error margin can be less than 5%. The current limiting circuit can also operate at up to 75 Volts minimum voltage across the terminals in a pulsed mode, with a pulse during of 8-40 mSec, and a pulse width of at least 4 mSec.

According to one embodiment, the current limiting circuitry's turn on time is configured to be at most 1 pS. In further embodiments, less turn-on times are also possible. Additionally, the current limiting circuitry exhibits at most 10 nA of leakage current per pin to substrate under 10 Volts.

Implanting multiple electrodes gives rise to risk of injury when the patient undergoes a defibrillation procedure or other procedures involving high-voltage sources. During the defibrillation procedure, a high voltage is applied to the patient's body. The voltage establishes a strong electrical field within the patient's chest, which can induce high voltage differences between electrodes, and, if a low-impedance circuit is formed, can result in high current density near the electrodes. Such high current density can cause injuries. Embodiments of the present invention provide a circuit that prevents high-density tissue current during defibrillation or other events involving high voltages in a multi-electrode pacing system. By using transistors that turn on for the regular pacing pulses and turn off during defibrillation, the circuit can effectively isolate the electrodes from the pacing wires during defibrillation and, therefore, prevent the formation of a low-impedance circuit through a patient's tissue. Furthermore, this circuit provides pass-through for regular pacing pulses without introducing significant voltage drop to the pacing signal, thereby facilitating a more power efficient pacing system.

Embodiments of the present invention can prevent injuries caused by high-density currents in a wide range of events involving high voltages. Such events include accidental electrocutions, shock treatments, and other medical procedures that apply high voltages to a patient's body. The circuit configurations disclosed herein facilitate protection against a high voltage source, such as a defibrillator, at a voltage at about 500 volts. In further embodiments, protection against a voltage greater than 1000 volts is possible.

Defibrillation is a technique used in emergency medicine to terminate ventricular fibrillation or pulseless ventricular tachycardia. A controlled electrical shock is applied to the patient's body to reset the electrical state of the heart, so that it may beat to a normal rhythm. The shock is applied through two electrodes, typically in the form of two hand-held paddles or adhesive patches. One electrode is placed on the right side of the front of the chest just below the clavicle, and the other electrode is placed on the left side of the chest just below the pectoral muscle or breast.

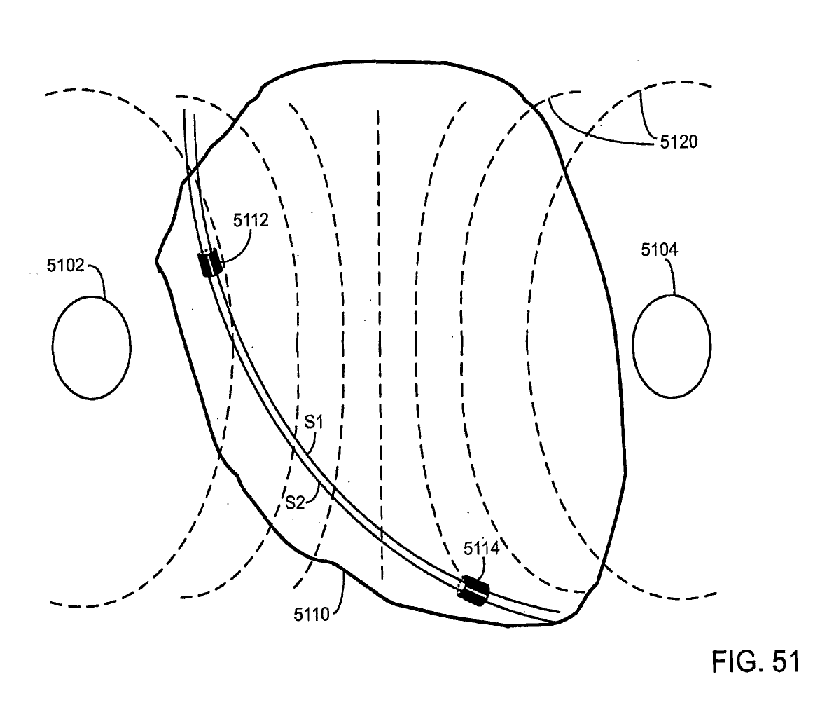

During defibrillation, a high-voltage pulse, typically at hundreds of volts, passes through the patient's upper body. This voltage results in an electrical field, which can induce a corresponding voltage on an electrode of an implanted pacing satellite. FIG. 51 illustrates an exemplary scenario where a defibrillation electrical field results in a voltage drop between two pacing satellites. A defibrillation pulse is applied through two defibrillation pads, 5102 and 5104. A corresponding electrical field, represented by the dashed equal-potential lines 5120, is present. Two pacing satellites, 5112 and 5114, which are coupled to two bus wires S1 and S2 respectively, are implanted in the patient's heart 5110. Because electrodes 5112 and 5114 are conductors, the electric field can induce a voltage on each electrode. The different locations of electrodes 5112 and 5114 can cause a voltage difference between satellites 5112 and 5114. If satellites 5112 and 5114, and bus wires S1 and S2 form a low-impedance circuit as part of the closed circuit which includes the defibrillation device, defibrillation pads, and heart tissue, high current density can be present near the electrodes on the satellites.

When a high-density current reaches tissue surrounding a pacing satellite, especially at focused points near an electrode, a resulting large electrical and heat surge can occur. Such an effect is similar to that used clinically for ablation proposes. However, in the case of inadvertent “ablation”-style damage to healthy tissue caused by high-density currents, serious tissue damage and destruction can occur. This is not good clinical practice, especially in a cardiac challenged patient, whose health could be further compromised by the procedure.

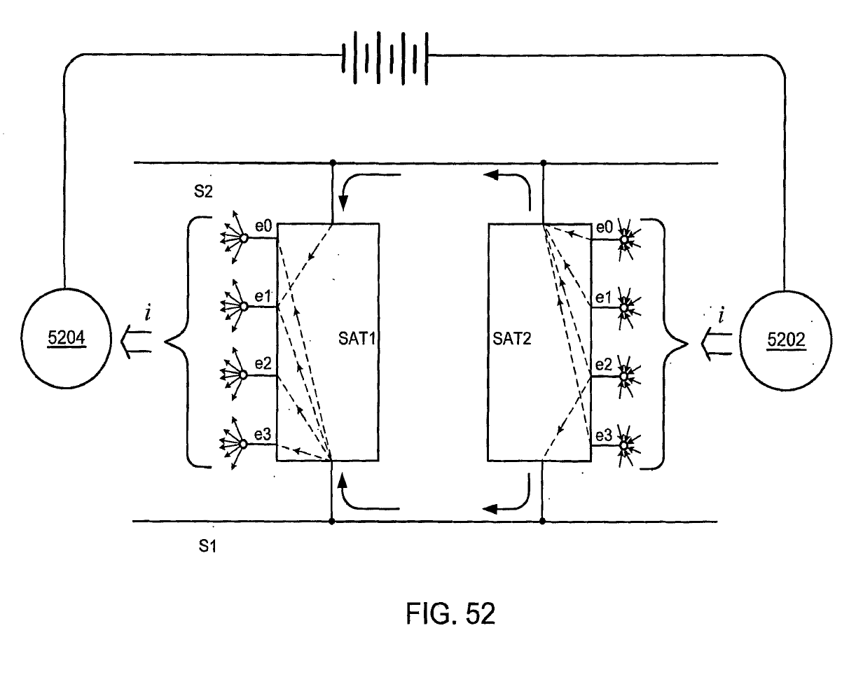

FIG. 52 illustrates an exemplary scenario where two pacing satellites without overcurrent protection allow a high-density current to pass through the tissue surrounding the electrodes during a defibrillation process. The defibrillation pulse is applied through two electrode pads, 5202 and 5204. Two pacing satellites, SAT1 and SAT2, are coupled to two bus wires, S1 and S2. Each satellite includes four electrodes, e0, e1, e2, and e3. An electrode on a given satellite is hereinafter referred to as SATi_ej, where i denotes the satellite index and j denotes the electrode index.

Assume that SAT2_e1 is selected and coupled to S1, and that the defibrillation field induces a high voltage of +60 V on all the electrodes on SAT2. This induced voltage can easily pass onto S2 all the electrodes and the switch circuits that are in a break-down state due to the induced high voltage. In addition, the induced voltage can also pass onto S1 through SAT2_e1, due to the diode/switch effect resulting from using a single MOSFET transistor as the output control switch. Hence, all the electrodes on SAT2 can be effectively coupled to S1 and S2. Note that when an internal transistor switch breaks down, the voltage passing through may experience a diode voltage drop. Nevertheless, such a voltage drop (for example, 0.7 V) is small compared with the magnitude of the induced high voltage such as +60 V.

The high voltage passed onto S1 and S2 further causes the switch circuits between the electrodes in SAT1 and S1 to break down. Therefore, all the electrodes on SAT1 can be effectively coupled to S1. Assume that one of the electrodes on SAT1, SAT1_e1, is already coupled to S2 as part of the configuration for regular pacing operation. As a result, the high voltage on S2 causes a current to flow through SAT1_e1, and the high voltage on S1 causes a current to flow through all four electrodes due to internal switch break down.

A low-impedance circuit is thereby formed through the electrodes on SAT2, the two bus wires S1 and S2, and the electrodes on SAT1. A current, which otherwise is a low-density tissue current disseminated through the tissue flowing from pad 5202 to pad 5204, is now concentrated at the electrodes on SAT2 and SAT1. This current can result in a high current density near the electrodes due to the small size of these electrodes. The high current density can injure the patient, for example, by over-heating the surrounding tissue. Hence, it is critically important to prevent formation of a low-impedance circuit through the multi-electrode pacing system during defibrillation.

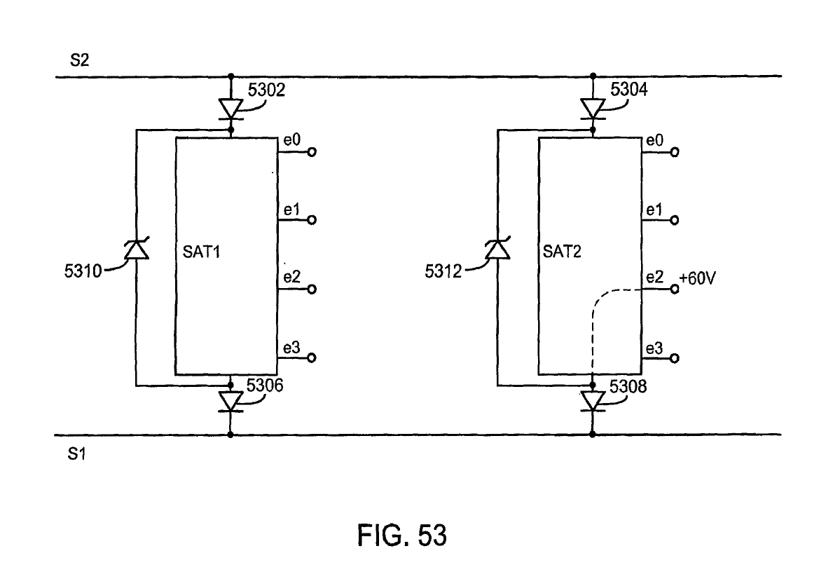

FIG. 53 illustrates an exemplary configuration of two pacing satellites where diodes are used to prevent the formation of a low-impedance circuit. Diodes 5302 and 5306 are placed between SAT1 and the two bus wires S2 and S1, respectively. Similarly, diodes 5304 and 5308 are placed between SAT2, and S2 and S1, respectively. Assume that a +60 V voltage is induced on the electrodes on SAT2 during defibrillation. The induced high voltage cannot reach S2 because diode 5304 is reverse biased. This voltage can only reach S1, through the electrodes and broken-down switch circuits (and an electrode already coupled to S1, if there is such an electrode).

However, the high voltage on S1 cannot reach SAT1 because this voltage causes diode 5306 to be reverse biased. As a result, SAT1 is isolated from S1 and a low-impedance circuit cannot be formed through the electrodes on SAT1. Without a low-impedance circuit, the defibrillation current flowing through any of the electrodes into the tissue is negligible.

When defibrillation is not performed, and when normal pacing is conducted through the satellites, S1 and S2 are used to carry the pacing signals. The configuration illustrated in FIG. 53 assumes that S2 carries a high-voltage signal and S1 operates as the return circuit for that signal. During pacing, the four diodes are forward biased, allowing the pacing signal to flow through the selected satellite. If S1 carries a high-voltage pacing signal and S2 provides the return circuit, the direction of the four diodes should be correspondingly reversed to allow the pacing signal to pass through.

Also included in the configuration shown in FIG. 53 are two Zener diodes, 5310 and 5312. These Zener diodes ensure that the rail-to-rail power-supply voltage provided by S2 and S1 to the satellites do not exceed a pre-determined value, which is the threshold voltage for these Zener diodes.

The use of diodes 5302, 5304, 5306, and 5308 prevents the formation of a low-impedance circuit during defibrillation. However, these diodes introduce undesired voltage drop during regular pacing. When forward biased, each diode typically introduces a forward voltage drop of 0.7 V. The total voltage drop introduced by diodes 5304 and 5308 between S2 and S1 can be as high as about 1.4 V. Such a voltage drop increases power consumption and causes the pacing system to be inefficient. In addition, diodes can take extra chip space, which is valuable in a satellite control chip confined to about 1 mm2.

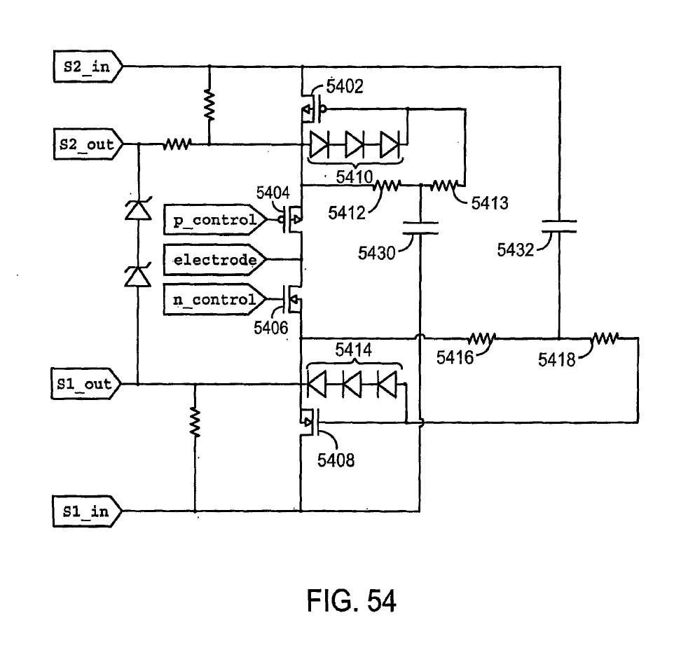

One embodiment of the present invention provides a circuit that isolates electrodes on different satellites during defibrillation, and introduces minimal voltage drop when the electrodes are used for normal pacing. FIG. 54 presents a schematic circuit diagram illustrating a configuration that uses transistors to isolate an electrode from a bus wire in accordance with an embodiment of the present invention.

This circuit provides four ports to interface with S1 and S2, S1_in, S1_out, S2_in, and S2_out. Two control signals, namely p_control and n_control, determine to which bus wire the electrode is to be coupled. p_control is fed to the gate of a PMOS transistor 5404. When p_control is at a low voltage, PMOS transistor 5404 is turned on, and, correspondingly, the electrode is allowed to couple to S2_in, subject to the state of a PMOS transistor 5402. If p_control is at a high voltage, PMOS transistor 5404 is turned off, and the electrode is isolated from S2.

Similarly, when n_control is at a high voltage, NMOS transistor is turned on and the electrode is allowed to couple to S1_in, subject to the state of an NMOS transistor 5408. If n_control is at a low voltage, NMOS transistor 5408 is turned off, and the electrode is isolated from S1.

PMOS transistor 5402 is located between S2_in and S2_out, and provides the necessary isolation during defibrillation and a pass-through with minimal voltage drop during regular pacing. When defibrillation is not applied, and when the satellite is between two pacing pulses, the gate, source, and drain of PMOS transistor 5402 are substantially at a low voltage, for example, 0V. When a high-voltage pacing pulse arrives from S2_in, the source voltage of PMOS transistor 5402 is sufficiently higher than the gate voltage thereof which is still at a low voltage. Hence, PMOS transistor 5402 is turned on.

This pacing pulse passes through PMOS transistor 5402 with little voltage drop (e.g., a few mV), and reaches the electrode, assuming that this electrode is selected to couple to S2 for pacing purposes. Resistors 5412 and 5413, and capacitor 5430 form an R-C feedback circuit that allows the gate voltage of PMOS transistor 5402 to rise sufficiently after the pacing pulse starts passing through. The increased gate voltage turns off PMOS transistor 5402 after a certain period, which can be adjusted by changing the values of the resistance and capacitance of the R-C feedback circuit to match the width of the pacing pulse. Therefore, PMOS transistor 5402 can turn on just long enough to allow the pacing pulse to pass through, and turn off afterwards.

During defibrillation, assume that the electrode is at an induced high voltage, for example, +60 V. This high voltage turns on PMOS transistor 5404 because the gate thereof is at a substantially lower voltage. However, this high voltage cannot pass through PMOS transistor 5402, because three forward biased diodes 5410 can quickly raise the gate voltage thereof to turn off PMOS transistor 5402. Even if each diode introduces a forward voltage drop of about 0.7 V, the gate voltage of PMOS transistor 5402 remains substantially higher than the source voltage thereof, and therefore PMOS transistor 5402 is turned off. The induced high voltage is isolated from S2_in and no low-impedance circuit can be formed. Note that the number of the feed-forward diodes 5410 can be adjusted, so that the circuit can provide a quick enough response to turn off PMOS transistor 5402 during defibrillation, and still allow PMOS transistor 5402 to be turned on for a sufficiently long period to allow a pacing pulse to pass through. Not that although the electrode is isolated from S2_in during defibrillation, the induced high voltage can still pass on to S1_in and reach another satellite. The isolation between the electrode and S1_in when S1_in is at a high voltage is provided by an NMOS transistor 5408.

NMOS transistor 5408, diodes 5414, resistors 5416 and 5418, and capacitor 5432 provide a similar protection. During regular pacing, NMOS transistor 5408 is temporarily turned on to allow the pacing pulse to pass through to S1. The R-C feedback circuit formed by resistors 5416 and 5418 and capacitor 5432 allows sufficient turn-on time for the pass through. During defibrillation, if S1_in is at an induced high voltage, diodes 5414 ensure that the gate of NMOS transistor 5408 is kept at a low voltage and that NMOS transistor 5408 is turned off to isolate the voltage on S1_in from reaching the electrode.

Note that two Zener diodes are placed between S2_out and S1_out. As mentioned above, when the electrode is at an induced high voltage, although the electrode is effectively isolated from S2_in, this high voltage can still reach S2_out and can further reach “downstream” pacing satellites, The Zener diodes ensures that the voltage between S2_out and S1_out does not exceed the corresponding Zener threshold voltage, thereby protecting the “downstream” pacing satellites from overvoltage.

In one embodiment, the resistors 5412, 5413, 5416, and 5418 each have a resistance of about 2,000 kΩ. In further embodiments, resistance greater than about 2,000 kΩ are possible. Capacitor 5430 and 5432 each have a capacitance of about 500 pF. In further embodiments, capacitance larger than about 500 pF is possible.

The layout for PMOS transistors 5402 and 5404, and the layout for NMOS transistors 5406 and 5408, each have a width-to-length ratio of 10,000. In a further embodiment, the lengths of these transistors are substantially 2 lambdas, and the widths of these transistors are substantially 20000 lambdas, based on the lambda based CMOS design rules. Note that one lambda is equal to one half of the “minimum” mask dimension, typically the length of a transistor channel. Other width-to-length dimensions and specific width or length sizes are possible.

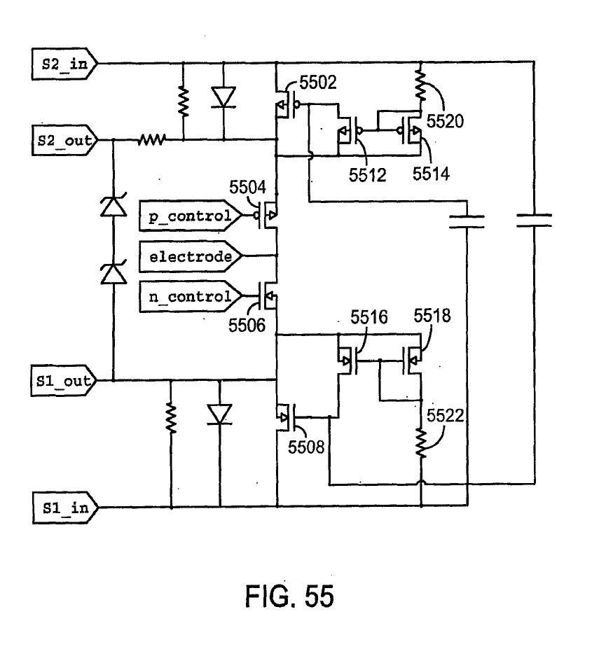

FIG. 55 presents a schematic circuit diagram illustrating a configuration that uses current mirrors to isolate an electrode from a bus wire in accordance with an embodiment of the present invention. The R-C feedback circuits as is shown in FIG. 54 may involve large capacitors which consumes chip space. Additionally, manufacturing resistors with MOSFET transistors (for example, with a squeezed, long gate region) may not produce an accurate resistance.

The circuit illustrated in FIG. 55 uses a current mirror to isolate the electrode from a bus wire during defibrillation. On the S2_in side, PMOS transistors 5512, 5514, and a resistor 5520 form a current mirror. When the electrode is at an induced high voltage, this voltage turns on PMOS transistor 5504 and reaches the current-definition branch of the current mirror, which includes PMOS transistor 5514 and resistor 5520. Resistor 5520 is chosen so that the current flowing through the current-definition branch toward S2_in is sufficiently small, for example, 1 mA. PMOS transistor 5512 is chosen so that the current flowing through itself is substantially larger (for example, 10 mA) than the current flowing in the definition branch. This configuration ensures that, when the electrode is at a high voltage, sufficient current can flow into and pull up the voltage of the gate of PMOS transistor 5502, so that PMOS transistor 5502 can turn off quickly.

During regular pacing, S2_in provides a voltage that turns on diode-connected PMOS transistor 5514 and produces a current through resistor 5520. As a result, PMOS transistor 5512 also produces a current flowing away from the gate of PMOS transistor 5502, which depletes the gate voltage thereof, turns on PMOS transistor 5502, and allows the pacing pulse to pass through.

The current mirror on the S1_in side, which includes NMOS transistors 5516 and 5518, and resistor 5522, functions in a similar way. During defibrillation, S1_in exhibits an induced high voltage resulting in a current flowing through resistor 5522 toward NMOS transistor 5518. NMOS transistor 5516, which is in the current-production side of the current mirror, is chosen so that the current produced therein is substantially larger than the current flowing through NMOS transistor 5518. Consequently, the charges stored in the gate of NMOS transistor 5508 are quickly depleted to turn off NMOS transistor 5508.

During regular pacing, S1_in provides a low-voltage return circuit that turns on diode-connected NMOS transistor 5518 and produces a current flowing through resistor 5522 toward S1_in. As a result, NMOS transistor 5516 also produces a current flowing into the gate of NMOS transistor 5508, which increases the gate voltage thereof, turns on NMOS transistor 5508, and allows the pacing pulse to pass through.

In one embodiment, resistor 5520 has a resistance of about 50,000Ω. Resistor 5522 has a resistance of about 1,000Ω. In further embodiments, other values of resistance are possible. The capacitors coupled to S2_in and S1_in each have a capacitance of 1 pF. In further embodiments, capacitance larger than 1 pF is possible.

In one embodiment, PMOS transistor 5514 has a width of about 100 lambdas and a length of about 2 lambdas. PMOS transistor 5512 has a width of about 1000 lambdas and a length of about 2 lambdas. PMOS transistors 5502 and 5504 each has a width of about 10,000 lambdas and a length of about 2 lambdas. NMOS transistor 5518 has a width of about 60 lambdas and a length of about 2 lambdas. NMOS transistor 5516 has a width of about 600 lambdas and a width of about 2 lambdas. Both NMOS transistors 5506 and 5508 each has a width of about 10,000 lambdas and a length of about 2 lambdas. Other values of width and length for these transistors are possible.

The layout for PMOS transistors 5502 and 5504, and the layout for NMOS transistors 5506 and 5508, each have a width-to-length ratio of 10,000. In a further embodiment, the lengths of these transistors are substantially 2 lambdas, and the widths of these transistors are substantially 20000 lambdas, based on the lambda based CMOS design rules. Note that one lambda is equal to one half of the “minimum” mask dimension, typically the length of a transistor channel. Other width-to-length dimensions and specific width or length sizes are possible.

### Off-Chip Capacitor

There is a need for capacitors that can be used on integrated circuits requiring large energy storage, or integrated circuits that require a bypass capacitor. One of the current employed methods to provide capacitance on an integrated, circuit chip is to integrate the capacitor onto the silicon itself. Embodiments of the present invention is related to pending PCT applications “Implantable Addressable Segmented Electrodes” PCT/US2005/046811 filed Dec. 22, 2005, and “Implantable Hermetically Sealed Structures” PCT/US2005/046815 filed Dec. 22, 2005, both of which are incorporated herein in their entirety by reference.

Manufacturing capacitors directly into the chip provides a straightforward manufacturing approach, and produces effective capacitance. However, these engineering designs have certain limitations. By example, relatively small amounts of capacitance is achieved at the cost of a very large percentage of space being occupied on the chip. In some cases, the loss of the chip space limits the availability of desirable additional circuitry, with its attendant additional features. In some instances, larger chips can be provided to limit this disadvantage. However, a larger chip may be impracticable due to the size constraints of the chip enclosure

An alternative approach to providing capacitance on an integrated circuit chip is to provide a discreet capacitor in the same package as the integrated circuit. Providing a discreet capacitor in the same package as the integrated circuit frees up space on the chip for other circuitry. Because additional circuitry with its attendant additional features can be incorporated into the chip, this approach has, the advantage over capacitors integrated directly into the chip. However, a discreet capacitor increases the complexity not only of the finished product but also of the assembly process, increasing product cost and stress risks. It also necessitates placing more than one component into a single package. Without a single unit, there are additional points of failure introduced into a device and the additional points of failure is not desirable.

A different approach in providing capacitance on an integrated circuit would be to attach the integrated circuit and capacitor onto one circuit board. The entire package would then be placed in a package. Again, this approach involves too many components to be practical in most cases.

It would be an important advancement in the art of micro-circuitry design if capacitance were available which did not occupy chip surface, but did not incur the design disadvantages of a discreet capacitor unit. There would be special applicability of such advancement to medical devices, with special advantages to implantable medical devices. The present invention provides, for this first time, these heretofore unavailable features.

The present invention exploits the surface of structures physically proximate to an IC chip to provide capacitance without a discreet capacitor unit. In one embodiment, the inside surface of a MEMS package used for hermetic sealing of a chip is employed to provide an energy storage capacitor. The advantage of this off-chip integrated capacitor innovation is that a sizeable capacitor is available to the system without the finished package being significantly larger or more complex.

In the present off-chip integrated capacitor innovation, the device effectively transforms the chip package itself into a capacitor. All that is added to the conventional chip package is a few layers of material to the inside of the device. Another associated device member, such as an electrode, can also be exploited to provide the off-chip integrated capacitor.

This off-chip integrated capacitor enjoys all the versatility of the bulkier, prior art capacitor, with additional advantages. For instance, the inventive off-chip integrated capacitor can be used for either energy storage or for a bypass, depending on the needs of the system.

Capacitance of the off-chip integrated capacitor can be adjusted by varying thickness, dielectric material, and other variables well known to one of ordinary skill in the art. For example, the capacitance of a capacitor constructed of two plane electrodes of area A at spacing d is about equal to the following:

$$C = {ε}_{0} {ε}_{r} \frac{A}{d}$$

where

∈₀ is the permittivity of free space

∈ᵣ is the dielectric constant of the insulator used,

C is the resulting capacitance.

Assuming a capacitor material with a surface area of about 1 μm², a dielectric constant of silicon dioxide, 3.9, and various spacing between capacitor planes, different capacitance values can be obtained. A thickness of 25 μm would achieve a capacitance of about 6.9 fF. Similarly, a thickness of 10 μm would achieve a capacitance of about 0.345 fF. Further, a thickness of 1 μm would achieve a capacitance of about 0.07 fF.

Capacitance can be achieved at the level of from about 0.07 to 6.9 fF, more specifically from about 0.2 to 1 fF, and most specifically about 0.4 fF.

The amount of charge is defined by:

Q=C·V

where

Q is the charge

C is the capacitance

V is the voltage stored on the capacitor.

One embodiment of the system provides reasonably robust capacity appropriately to the application of the device it serves at a voltage from about 5 to 10 volts. For example, at 5V stored on the capacitor, the off-chip integrated capacitor can hold charge from about 0.35 to 35 fC, more specifically from about 1 to 5 fC, and most specifically about 2 fC.

The inventive integrated off-chip capacitance design enjoys several advantages over prior art capacitance approaches. First, there is an enormous gain in chip “real estate” without the risks involved in losing the functionality of a capacitor. Indeed, the capacitor gained by the inventive construct is essentially integrated and more reliable than those of previous methods. An additional advantage is that this improved design is lower cost than prior art approaches. Added advantages are that the simpler assembly speeds production time, and limits defects and stresses introduced by undue handling.

While one implementation of the present invention is described below, the present invention can utilize all the internal surfaces of a MEMS packaging unit. For instance, via and contact openings can be used to attach the capacitor structure to an IC. This IC can rest on the top or bottom surface of the package.

In one embodiment, the inside surface of a MEMS package used for hermetic sealing of a chip is employed to provide an energy storage capacitor. The capacitor can be produced by depositing a thin layer of highly porous material to optimize the surface area of the capacitor. Then, a conductor material is deposited to form one plate of the capacitor. Deposited over this is an insulator to create the dielectric for the capacitor. This insulator would preferably have a high degree of porosity. Having this material be a poor dielectric is a desired property in many embodiments of the integrated off-chip capacitance device. As a final step, conductor material is deposited to form the second plate of the capacitor.

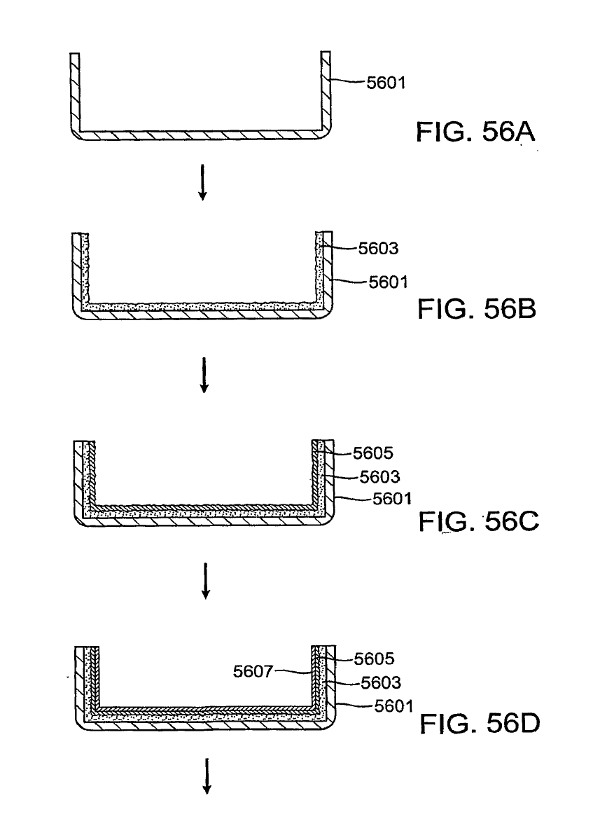

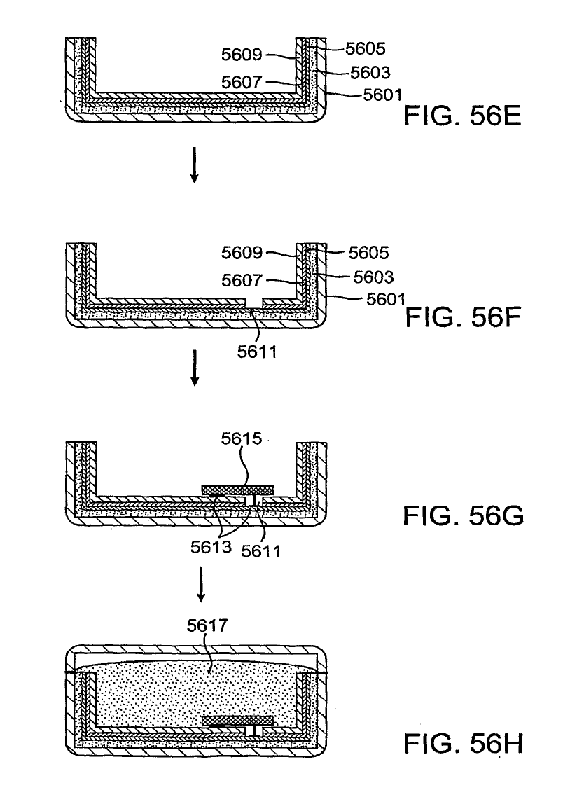

To illustrate, FIG. 56A provides a flow diagram of one example of the early stages of manufacturing an integrated off-chip capacitance device. The first step A shows a cross section of an empty cavity, the chip package 5601, prior to the insertion of the IC chip. Chip package 5601 will typically be constructed of silicon, but other materials are also useful in this regard.

FIG. 56B shows the same cavity of chip package 5601 having been coated with a layer of insulator 5603. Insulator 5603 typically has a very high degree of porosity. This quality keeps the surface area of insulator 5603 high as compared with the physical surface area of the cavity in chip package 5601 in which insulator 5603 resides. The material used to produce insulator 5603 will typically have a poor dielectric property. This feature of the material used to produce insulator 5603 avoids the potential creation of a parasitic capacitor.

FIG. 56C illustrates application of a conductive layer 5605 on top of insulator 5603. This fabrication step forms the bottom plate of the final assembled integrated off-chip capacitor.

FIG. 56D illustrates the addition of a layer of dielectric material 5607. FIG. 56E shows the addition of the second conductive layer 5609. The addition of second conductive layer 5609 provides the second plate of the integrated off-chip capacitor. With this addition, capacitor component of the device is complete.

FIG. 56F shows opening 5611 which is created on the top layer of the new capacitor, that is second conductive layer 5609. Opening 5611 is provided through the dielectric to the first plate of the capacitor.

In FIG. 56G, IC device 5615 can be attached to second conductive layer 5609 through pad 5613. Conductive layer 5605 can be attached to IC device 5615 through pad 5613.

FIG. 56H shows the final processing steps for the integrated off-chip capacitor. An insulator 5619 is poured over the entire construct shown in FIG. 56G. Insulator 5617 serves to pot the integrated off-chip capacitor and IC device 5615 with which it is associated.

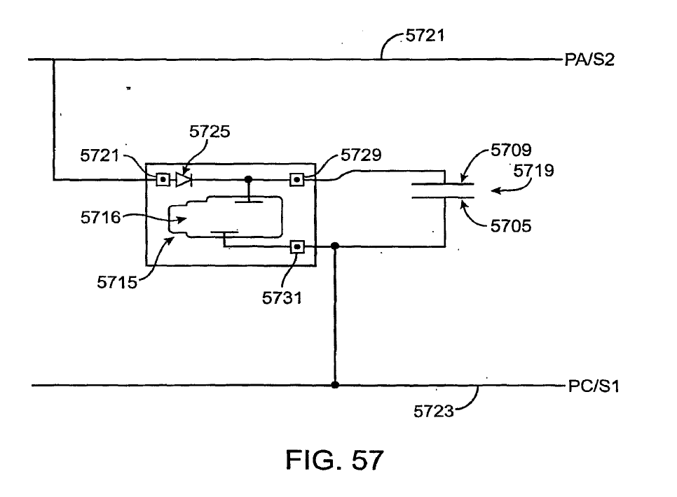

In one embodiment of the present invention, FIG. 57 illustrates one way IC device 5715 can be attached to integrated off-chip capacitor device 5719. FIG. 57 includes anode wire 5721 and cathode wire 5723. Anode wire 5721 can be attached to IC device 5715 through pad 5727. IC device 5715 can be attached to integrated off-chip capacitance device 5719 through pads 5729 and 5731. Electronics 5716 are powered by integrated off-chip capacitance device 5719.

Integrated off-chip capacitance device 5719 is typically charged through a pacing pulse that runs from anode wire 5721 to rectifying diode 5725, through integrated off-chip capacitance device 5719, and back through cathode wire 5723. The pacing pulse is rectified through rectifying diode 5725 before being stored on integrated off-chip capacitance device 5719. Integrated off-chip capacitance device 5719 can add from a few nano farads to a few tens of nano farads capacitance. These levels easily provide adequate power for devices which operate in burst mode.

### Implantable On-Chip Capacitor

There is a need for capacitors that can be used on integrated circuits requiring large energy storage, or integrated circuits that require a bypass capacitor. One of the currently employed methods to provide capacitance on an integrated circuit chip is to integrate the capacitor onto the silicon itself.

Manufacturing capacitors directly into the chip provides a straightforward manufacturing approach, and produces effective capacitance. However, these engineering designs have certain limitations. By example, relatively small amounts of capacitance are achieved at the cost of a very large percentage of space being occupied on the chip. In some cases, the loss of the chip space limits the availability of desirable additional circuitry, with its attendant additional features. In some instances, larger chips can be provided to limit this disadvantage. However, a larger chip may be impracticable due to the size constraints of the chip enclosure.

An alternative approach to providing capacitance on an integrated circuit chip is to provide a discreet capacitor in the same package as the integrated circuit. Providing a discreet capacitor in the same package as the integrated circuit frees up space on the chip for other circuitry. Because additional circuitry with its attendant additional features can be incorporated into the chip, this approach has an advantage over capacitors integrated directly into the chip. However, a discreet capacitor increases the complexity not only of the finished product but also of the assembly process, increasing product cost and stress risks. It also necessitates placing more than one component into a single package. Without a single unit, there are additional points of failure introduced into a device and the additional points of failure are not desirable.

A different approach in providing capacitance on an integrated circuit would be to attach the integrated circuit and capacitor onto one circuit board. The entire package would then be placed in a package. Again, this approach involves too many components to be practical in most cases.

It would be an important advancement in the art of micro-circuitry design if capacitance were available which did not occupy chip surface, but did not incur the design disadvantages of a discreet capacitor unit. There would be special applicability of such advancement to medical devices, with special advantages to implantable medical devices. The present invention provides, for the first time, these heretofore unavailable features.

The present invention exploits the surface of structures physically proximate to an IC chip to provide capacitance without a discreet capacitor unit. In one embodiment, the passivation layer or outer surface used for hermetic sealing of a chip is employed to provide an energy storage capacitor area. The advantage of this implantable on-chip capacitor innovation is that a sizeable capacitor is available to the system without the finished package being significantly larger or more complex.

In the present implantable on-chip capacitor innovation, the device effectively transforms the chip package surface into a capacitor. All that is added to the conventional chip package is a few layers of material to the outside of the device. If hermetic sealing is not required for an application, the implantable on-chip capacitor can be deposited on a dielectric that separates the electrodes from the chip.

The inventive implantable on-chip capacitor uses capacitive materials deposited on the outer surface of a protective layer that surrounds a circuit chip. When the structure comes in contact with ionic fluid from the body it provides a conduction path between the electrodes. The implantable on-chip capacitor provides a very high capacitance while maintaining a small size.

In one embodiment, the implantable on-chip capacitor is used in the system described in PCT application “Pharma-Informatics System” PCT/US2006/016370, filed Apr. 28, 2006, hereby incorporated by reference in its entirety. The implantable on-chip capacitor can be placed on the outer surface of a chip which can be inside or attached to a pill containing a pharmaceutically active agent. When the pill is ingested, the implantable on-chip capacitor can use the stomach fluids to create an electrolytic capacitor. In doing so, the implantable on-chip capacitor not only abandons the traditional hermetic sealing of a capacitor, but also the packaged electrolytic fluid.

This implantable on-chip capacitor enjoys all the versatility of the bulkier, prior art capacitor, with additional advantages. For instance, the inventive off-chip integrated capacitor can be used for either energy storage or for a bypass, depending on the needs of the system.

Capacitance of the implantable on-chip capacitor can be adjusted by varying thickness and other variables well known to one of ordinary skill in the art. For example, the capacitance of a capacitor constructed of two plane electrodes of area A at spacing d is about equal to the following:

$$C = {ε}_{0} {ε}_{r} \frac{A}{d}$$

where

∈₀ is the permittivity of free space

∈ᵣ is the dielectric constant of the insulator used,

C is the resulting capacitance.

The amount of charge is defined by:

Q=C·V

where

Q is the charge

C is the capacitance

V is the voltage stored on the capacitor.

The electrodes of the implantable on-chip capacitor can be designed in a variety of configurations. In one embodiment of the implantable on-chip capacitor, the electrodes which make up the capacitor can be formed into columns. This allows the implantable on-chip capacitor to utilize the added surface area that is added from the taller columns. The implantable on-chip capacitor electrodes can be placed on opposite sides of the substrate. This provides a different form factor which can be useful in certain applications.

The implantable on-chip capacitor can also be put into parallel. The implantable on-chip capacitor can contain more than one capacitor. Multiple implantable on-chip capacitors can be connected in series. Having the capacitors placed into series can allow for higher voltages or it can allow the voltage to be spread out over more electrodes so that they hold less voltage individually. This can be an advantage for applications that require larger voltages, but need to do so without exceeding a maximum voltage on a given capacitor e.g. breaking the water window or harming the host.

In one embodiment of the implantable on-chip capacitor the electrodes are covered by a porous material that will allow the bodily fluids to reach the electrode plates. The highly porous material ensures that fluid remains in contact with the electrodes and provide protection to the surface of the electrodes from any debris that may be in the body. This provides that nothing inside of the body will interfere with the conduction path while still allowing the capacitor to function.

The implantable on-chip capacitor uses capacitive materials deposited on the outer surface of a structure, which come in contact with the surrounding ionic fluid, such as that found in the body. This design provides a very high capacitance while maintaining a small overall device size, making it ideal for implantable medical devices. The inventive implantable on-chip capacitor can be used for either energy storage or for a bypass, depending on the needs of the system.

The implantable on-chip capacitor enjoys the capabilities of an electrolytic capacitor without the size previously required to design such a device. The implantable on-chip capacitor is located on the outside of the chip and requires no enclosure. Since there is no packaging taking up excessive room on the chip, the capacitor can be smaller while providing the same capacitance. Because the implantable on-chip capacitor utilizes the surrounding naturally occurring ionic fluids it does not require an ionic solution to be enclosed in a can or container. This conserves even more room for the capacitor and allows for a smaller device size.

In one embodiment, the implantable on-chip capacitor can be utilized in the system described in PCT application “Pharma-Informatics System” PCT/US2006/016370, filed Apr. 28, 2006, hereby incorporated by reference in its entirety. The inventive implantable on-chip capacitor can be placed on the outer surface of a pill containing a pharmaceutically active agent. When the pill is ingested into the body, it comes in contact with the stomach fluids which act as the ionic fluid required to operate the capacitor. The small profile of the capacitor is ideal for this and many other applications such as heart, spinal, ear, retina, stomach and gastric implants.

The implantable on-chip capacitor can be utilized in the system described in U.S. Provisional application “Void-Free Implantable Hermetically Sealed Structures” 60/791,244, filed Apr. 12, 2006, hereby incorporated by reference in its entirety. For applications requiring hermetic sealing, the implantable on-chip capacitor can be deposited on the outer surface, connected to circuit contacts which protrude through the hermetic sealing.

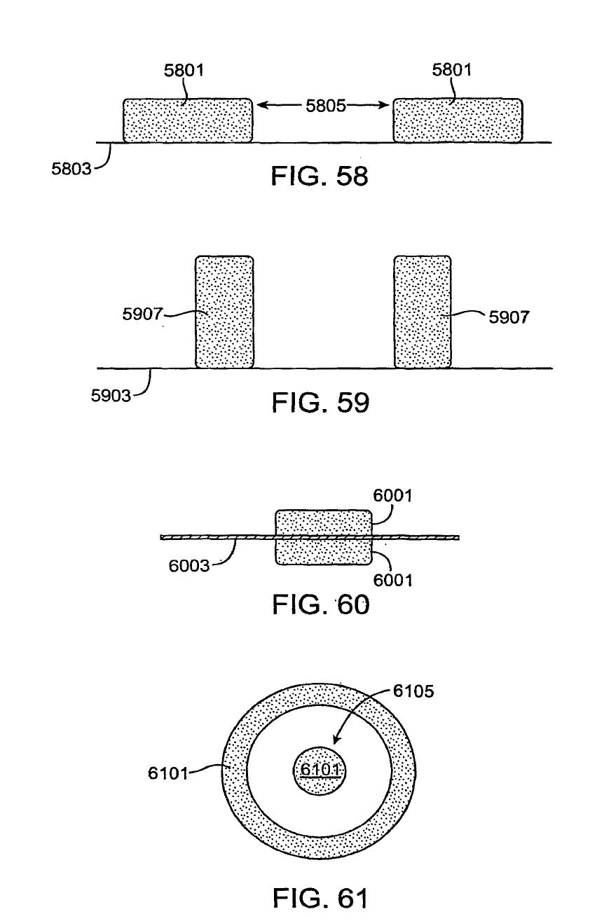

FIG. 58 shows one embodiment of the implantable on-chip capacitor in which the porous electrode material 5801 is deposited in a side by side, coplanar fashion on substrate 5803 with appropriate areas and a separation 5805 between the two. Separation 5805 can be about 0.25 to about 10.0 μm, more specifically about 3.0 to about 8.0 μm, and most specifically about 5.0 μm.

FIG. 59 shows another embodiment where the electrodes 5907 are formed as columns. This arrangement gives the benefit of the added surface area on the sides of the columns. Alternatively, the electrodes can be deposited in many other shapes which can conform to the surface of the circuit chip. The separation can be about 0.25 to about 10.0 μm, more specifically about 3.0 to 8.0 μm, and most specifically about 5.0 μm.

FIG. 60 shows an embodiment of the implantable on-chip capacitor in which the electrodes 6001 can be positioned on opposite sides of the substrate 6003. This provides a different form factor which can be advantageous for applications requiring a narrow, elongated profile.

FIG. 61 shows another embodiment of the implantable on-chip capacitor in which the electrode material 6101 is formed into concentric circles. The separation 6105 can be about 0.25 to about 10.0 μm, more specifically about 3.0 to about 8.0 μm, and most specifically about 5.0 μm. In another embodiment the electrode material 6101 is put into series by forming more concentric circles.

The electrode material 6101 can be made from any capacitive material. In biological applications any material which is safe for use in the body can be used. Platinum iridium is a good choice for use in the implantable on-chip capacitor because of its high capacitance and it is well established as an implantable material.

Alloys and other inert substances can also be potential materials. A material can be selected which can be deposited in a relatively thick and porous layer. Using cathodic arc deposition can provide the surface area and porosity that is needed to produce the large capacitance. Electrode material 6101 can be about 2.0 to about 200 μm thick, more specifically about 10 to about 40 μm thick, and most specifically about 15 to about 30 μm thick. Metals that can be oxidized can also be a good choice for the electrode material 6101. Titanium can be deposited via cathodic arc in its pure form to provide capacitance. A titanium oxide surface would provide a passivation layer to the implantable on-chip capacitor. The oxidized layer provides protection to the electrodes 6101 diminishing the drain of the implantable on-chip capacitor. The passivation layer also provides more protection from accidental discharge. Tantalum is another material that can be deposited via cathodic arc and oxidized. Titanium oxide and tantalum oxide can be deposited via cathodic arc deposition

Other materials that can be used as the electrode material 6101 include, but are not limited to, micro and nano-porous oxides, nitrides, carbides, oxynitrides and carbonitrides of the platinum group materials such as PtOx, IrOx, PdOx, OsOx, PhOx, PtN, IrN, PdN, RhN, AuN, PtC, IrC, PdC, AuC, PtON, PdON, IrON, RhON, PtCN, PdCN, IrCN, RhCN. The capacitor can also include porous, micro-porous and nano-porous compounds of TiO2 and Al2O3, TiON, AlON, TiC, AlC, TiCn, AlCN. TiCN, AlCN.

Using electrode material 6101 in an ingestible device application requires it to be reasonably physically, mechanically and chemically stable and robust since it will be swallowed, but will not need to have an exceedingly high mechanical strength. The implantable on-chip capacitor'needs to survive for the short period of time while it travels to the stomach and is activated by the stomach fluids.

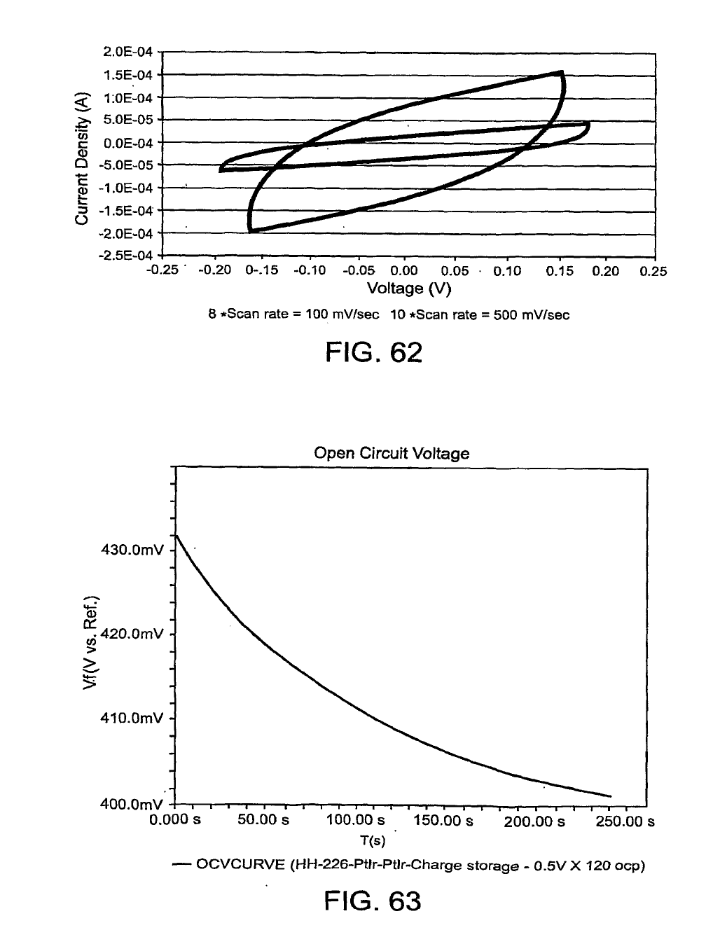

FIG. 62 shows data from experiments performed by some of the present inventors, a capacitor was manufactured where both electrodes were made of platinum iridium. The electrodes had an area of about 7.1 mm² and carried a capacitance of 3.44 mC/cm². The capacitance was determined by scanning the voltage at different rates and measuring how much current flowed through the capacitor. The experiment was performed in a voltage range of about −0.2V to about 0.2V at two different scan rates.

The capacitance was calculated by dividing the current by the change in voltage over time.

A similar experiment was performed with a platinum iridium implantable on-chip capacitor over a much larger voltage range. In that configuration the capacitor yielded a capacitance of about 10 mC/cm².

The implantable on-chip capacitor can yield a capacitance of about 0.5 to about 50 mC/cm², more specifically about 1 to about 25 mC/cm², and most specifically about 3 to about 10 mC/cm².

FIG. 63 is a graph demonstrating the voltage retention rate of a platinum iridium-platinum iridium battery that has an area of 7.1 mm². The implantable on-chip capacitor was charged at 0.5V for 120 seconds and then shut down to see how well it retained the half volt. As can be seen from the graph, the implantable on-chip capacitor has a bit of inefficiency as it starts at about 432 mV. Over about 4 minutes, the voltage only drops to about 402 mV. If the capacitor was made out of a material such as titanium with an oxidized passivation layer the self discharge would be much smaller.

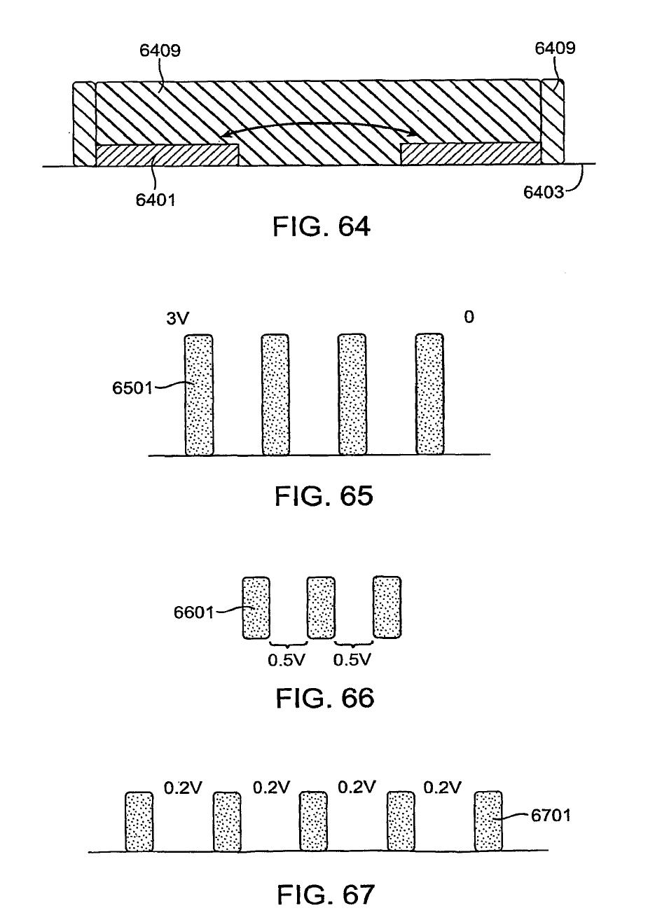

FIG. 64 shows an embodiment of the implantable on-chip capacitor with electrode material 1 on substrate 6403 covered by a porous material 6409. The porous material 9 will retain the fluid and prevent the surfaces of the electrode material 6401 from being obstructed by debris in the body. The conduction path can still pass between the two electrodes 6401 through the porous material 6409.

The porous material 6409 can be made using a number of materials such as titanium dioxide. Titanium dioxide is an ideal substance because it can be applied in a very porous manner using cathodic arc deposition and it will not dissolve or corrode in the body.

Other porous materials such as cellulose acetate or a porous polyethylene that are able to hold liquid can also be used. Porosity is the most important physical aspect of the porous material 6409 since the liquid must be able to penetrate to the electrode material 6401. A minimum thickness needs to be applied so that even when the pores are clogged with debris there is still liquid trapped around the electrodes 1. The porous material would be about 5 to 75 μm thick, more specifically about 15 to 40 μm thick and most specifically about 20 to 30 μm thick. The porous layer can be applied to any of the electrode configurations discussed above.

FIGS. 65, 66, and 67 show the electrode materials 1 being put in series. When a pair of electrodes is put in an aqueous solution, such as those present in the body, they cannot store more than about 1 to 1.2 volts before they begin to break down water. When a higher voltage is desired, the inventive implantable on-chip capacitor can be put in series. Putting the implantable on-chip capacitors in series allows the voltage to be spread out between the electrodes so that no more than 1 or 1.2 volts will be stored between any two electrodes and therefore would not break the water window. Each of the electrodes can be suitably isolated from the others in order to avoid higher voltages.

By example, FIG. 65 shows how a series of the inventive implantable on-chip capacitors can store a total charge of 3V without breaking the water window. FIG. 67 provides a design with the ability to use five electrodes 1 in order to decrease the voltage across each electrode and make it safer for certain biological uses. In the case of FIG. 67, there are four 0.2V charges being stored between the electrodes 1 resulting in a 0.8V total charge.

In another embodiment the inventive implantable on-chip capacitor can be enclosed in a membrane filled with an ionic fluid. This would allow the implantable on-chip capacitor to operate without the need of bodily fluids. The application of such a membrane would be useful in the event that no bodily fluids are present, or are present on an irregular basis.

### Data-Clock Recovery

In some embodiments of the inventive control circuitry; the control circuit is attached to the controller through only two wires, S1 and S2. There may be several control circuits attached in parallel to the same two bus wires. Each control circuit, in turn may control one or more effectors. The control circuit configures the effectors to be coupled to S1, S2, or a neutral high impedance state. A signal can then be sent through S1 or S2 to each connected effector, or a signal can be received from the effector. With only two wires handling all communication, powering, and signals to and from the effectors, there will be a wide range of signals present on bus wires S1 and S2 at any given time. A data encoding scheme is provided that accurately delivers a command to the controller in a manner that will not be confused with other signals that may be present. An efficient encoding scheme and decoding circuit is provided that will accomplish this goal, while at simultaneously obtaining the clock and generating the power from the same signal.

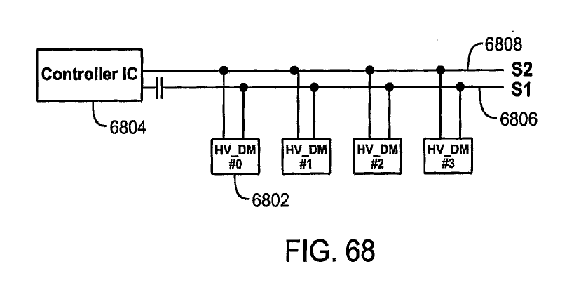

FIG. 68 shows an embodiment of the present invention, in which multiple control circuits 6802 are each connected to controller circuit 6804 through conductors 6806 and 6808, all of which may be implanted or inserted in the body. Each control circuit 6802 may be individually addressable by controller circuit 6804. Each control circuit 6802 can configure the state of one or more effectors which may be locally connected. Conductors 6806 and 6808 can be used to send commands and power to control circuits 6802, send signals through the associated effectors, and receive signals back from the effectors and control circuits 6802. In the case of a pacemaker lead, the control circuits 6802 may be connected to one or more electrodes. The electrodes may be configured to pace or sense, and the pacing pulses and sensed signals may travel on conductors 6806 and 6808.

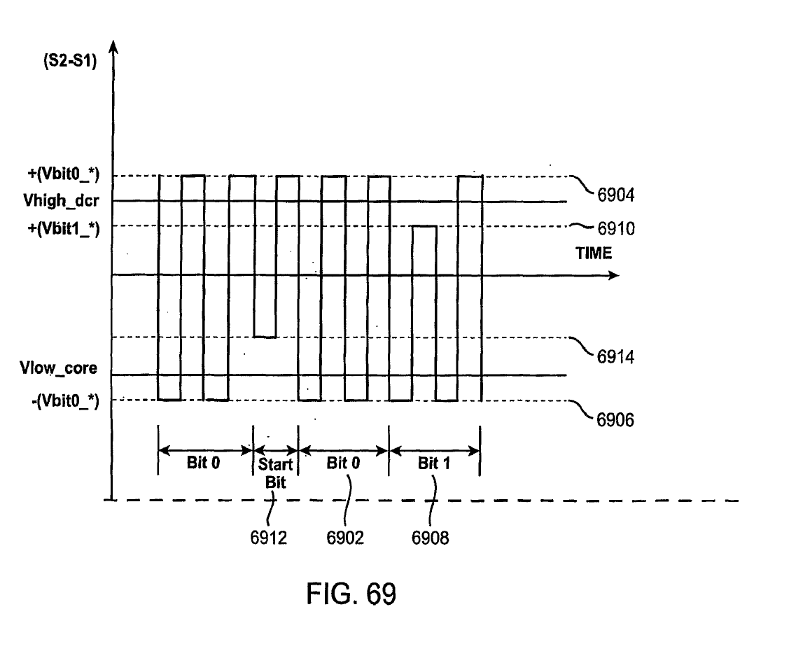

The inventive DCR circuit has the capacity to decode data which is encoded so that it can be distinguished from any other signal that may be present on the conductors. It also provides that the clock and power can be extracted from the data stream. FIG. 69 shows one embodiment of the data encoding scheme. The waveform shown represents the differential voltage signal across S1 and S2, which are conductors 6806 and 6808. In this embodiment, the signal used is S2−S1. A bit 0 6902 is represented with two full cycles of a square wave, going up to high voltage +Vbit0 6904 and down to low voltage −Vbit0 6906. A bit 1 6908 is represented with one cycle which goes up to a lower high voltage +Vbit1 6910 and down to the same low voltage −Vbit0 6906, followed by a second full cycle which goes up to +Vbit0 6904 and down to −Vbit0 6906. A start bit 6912 is represented by only one cycle of a square wave, which goes down to a higher low voltage −Vstart 6914 and up to the full high voltage 6904. In one embodiment, voltage +Vbit0 6912 can be about +4V, the voltage +Vbit1 6910 can be about +1V, voltage −Vbit0 6906 can be about −4V, and voltage −Vstart 6914 can be about −1V. This encoding scheme is merely an example of various approaches available using the present inventive embodiment. For example, any voltage values or assignment of bits can be used.

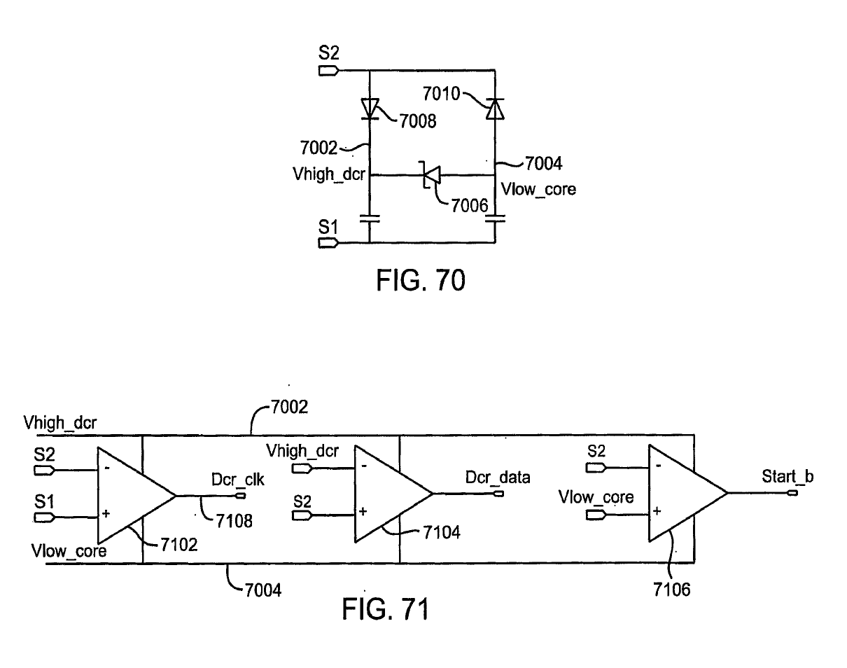

The power supply and reference voltages are both produced by the circuit in FIG. 70. Vhigh_dcr 7002 is one diode drop below S2. Vlow_core 7004 is one diode drop above S2. Zener diode 7006 has a 5V breakdown voltage and maintains a 5V difference between Vhigh_dcr 7002 and Vlow_core 7004. Using the example above, and assuming diodes 7008 and 7010 have a breakdown voltage of 1V, when the differential voltage between S2 and S1 goes high to 4V, Vhigh_dcr will be at 3V and Vlow_core will be at −2V. When the differential voltage goes low to −4V, Vlow_core will be at −3V and Vhigh_dcr will be at 2V. Vhigh_dcr and Vlow_core can then be used as both a power supply and a reference for deciding whether each bit is a 1, 0 or start bit.

FIG. 71 shows an embodiment of how the control circuit extracts the bits and the clock signal from the incoming signal. There are three comparators 7102, 7104, and 7106, each of which is powered by Vhigh_dcr 7002 and Vlow_core 7004. Comparator 7102 compares S2 6808 to S1 6806. This gives the clock signal Dcr_clk 7108, which can be used in the decoding of bits, and used by other circuit blocks.

Comparator 7106 compares Vlow_core to S2. Using the clock for symbol timing, the circuitry can determine when a start bit occurs, by identifying when the low period of the square wave is above Vlow_core. Once the start bit is found, the following bits are decoded and will be the command.

Comparator 7104 compares S2 to Vhigh_dcr. This information can be used by determining from the first high period of each symbol period whether the bit is a 1 or a 0. If the voltage of the first high period is above Vhigh_dcr, it is decoded as a bit 0. Alternatively, if the voltage of the first high period is below Vhigh_dcr, it is decoded as a bit 1.

Two cycles are used for each bit, whether it is a bit 0 or a bit 1, with the second cycle always returning to the high value. This step recharges the power supply Vhigh_dcr 7002. If only one cycle is used and a series of bit 1's are sent, the voltage will not go above Vhigh_dcr and the power supply will droop. Since Vhigh_dcr also serves as a reference, when Vhigh_dcr droops below the high voltage of a bit 1, there is an error and a bit 1 would be decoded as a bit 0. By always returning to the full high voltage, the power supply is restored. There is some fluctuation in the level of Vhigh_dcr, but it always remains in between +Vbit0 and +Vbit1, providing for accurate decoding of the bits.

This scheme allows a bit 0, a bit 1, and a start bit to be sent at the same frequency, while powering the decoding circuitry with the data signal itself.

### Wake-Up Circuit

One challenge when designing implantable devices is to limit their power consumption as much as possible. The standard approach of changing or recharging the battery of an implanted device can be risky and expensive, and often requires a surgical procedure to replace the battery. In one embodiment, the inventive circuitry greatly reduces power consumption by including a sleep mode which turns certain blocks off when they are not needed. In an additional embodiment, a sleep command can be sent during normal communication. This approach which will tell the circuitry to power down certain blocks.

A challenge comes when the circuit needs to wakeup. In some embodiments, the wake-up circuit is attached to two bus-wires, S1 and S2, which are the circuit's only means of communication with other components. In additional embodiments, the same bus wires are used to send command signals to other control circuits, and used to send and receive signals to and from the attached effectors. As a result of these innovations, there can be a wide range of voltage signals on S1 and S2 at any given time.

It is useful to have a wakeup circuit that can respond to a specific wakeup signal, but will not cause false wakeup when any other signals are present. A wakeup signal may be chosen which is unique from any other signal which will be present on the bus wires, but which can be detected by the wakeup circuit.

In one embodiment, the wakeup signal is chosen in a specific frequency range in order to trigger the wakeup circuit. In another embodiment, the voltage is selected at a certain level. In other embodiments, both the frequency and voltage are in a certain range in order to wakeup the circuitry. In yet other embodiments, a certain number of pulses are sent within a specified amount of time to trigger the wakeup circuit.

For the purposes of demonstrating an embodiment of the inventive circuit, consider the example described above of a control circuit which is attached to two bus wires, S1 and S2. There may be several control circuits attached to the same bus wires, each individually addressable by the controller IC. Normal communication signals may be transmitted at a nominal frequency of 1 MHz, and an amplitude of 4V. Since S1 and S2 are also used to send signals too and from the effectors associated with each control circuit, the voltage level on S1 and S2 will vary. In the example of a pacing lead used for sending pacing pulses to the heart, pacing pulses of up to about 10V may be present on S1 and S2, but would be sent as low frequency pulses. For this example a wakeup signal is chosen which is a square wave with an amplitude of +/−9V, and is at a frequency of about 500 kHz for a few cycles followed by a few cycles at 1 MHz. The reasons for this will become apparent upon consideration of the following description.

Because S1 and S2 are not used strictly for communication, the voltage present may fall in between the power supply voltages, Vhigh and Vlow, used by the circuitry. This power supply is typically held by a capacitor, as in FIG. 70. If the signal on S1 and S2 is allowed to pass through to the rest of the circuitry when it is between Vhigh and Vlow, and therefore not a logic 1 or 0, then the power supply will drain very quickly. For example, if an inverter is powered by Vhigh and Vlow, and the input signal is in between Vhigh and Vlow, it will turn on both transistors in the inverter, creating a DC pass from Vhigh to Vlow, quickly depleting the capacitor charge.

At the same time, the internal supply is moving around. Vhigh and Vlow are produced in a similar manner to Vhigh_dcr and Vlow_core in FIG. 70. Vhigh is one diode drop below S2, while Vlow is one diode drop above S1. In between Vhigh and Vlow is a 5V Zener diode. For example, if the differential voltage between S1 and S2 goes up to +9V, Vhigh will be at +8V and Vlow will be 5V below that at +3V. If the differential voltage goes down to −9V, Vlow will be at −8V, and Vhigh will be 5V above that at −3V. Therefore, a signal is achieved which tracks the supply so that the signal that reaches the circuit does not end up in the middle of the supply, draining the charge. Any voltage above 5V can be tolerated because that will recharge the supply.

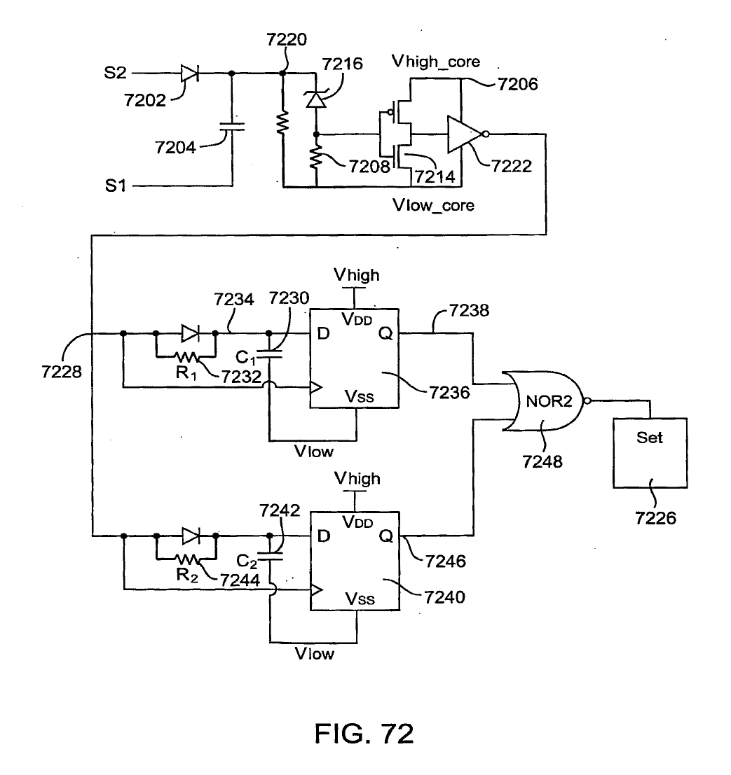

FIG. 72 shows one embodiment of a circuit that ensures that every signal that passes through to the rest of the wakeup circuitry will be in the correct logic form. There is diode 7202 and capacitor 7204 between S1 and S2. Whenever there is a voltage on the line, it is charging capacitor 7204. The capacitor acts as a supply for the rest of the circuit. Supply voltage Vhigh_core is connected to node 7206 and Vlow_core is connected to node 7208. Node 7210 is the input to transistors 7212 and 7214, which are arranged as an inverter.

As long as S1 and S2 are carrying a voltage signal below 5V, Zener diode 7216 will not breakdown, so the voltage at input 7210 will be pulled down toward Vlow at node 7208 through resistor 7218. In that case, input 7210 will be at a digital zero and will not drain the current from Vhigh to Vlow. This is useful in situations such as the above example, where communication is carried out at +/−4V. If commands are sent during sleep mode, they will not activate wakeup.

When a wakeup signal is sent, a higher voltage is used. For a wakeup signal of +/−9V, the first cycle the signal goes to +9V, S2 will be at +9V, S1 will be at 0V, and it charges capacitor 7204. Then S2 will go down to −9V, which will pull Vlow_core 7208 down to −8V. With node 7220 being held at +9V by capacitor 7204, there is enough voltage difference to trigger Zener diode 7216. The input 7210 to the inverter then becomes 9V−5V=4V. Since Vhigh_core is one Zener diode above Vlow_core, when Vlow_core is pulled down to −8V, Vhigh_core will be −3V. Since input 7210 is higher than Vhigh_core in that case, input 7210 becomes a logic 1. With another inverter 7222 at the output of the inverter made up of transistors 7212 and 7214, the logic 1 is essentially passed through to the next circuit.

After the first cycle, the input 7210 tracks the power supply and the differential signal S2−S1 will be passed on as long as it is fluctuating between +/−9V. It is possible that a signal which is not the wakeup signal, such as a pacing pulse, would pass through this portion of the circuit. Because of this, there can also be a portion of the circuit which distinguishes the signal based on frequency.

In order for the wakeup command to be issued, there must be a pulse sent to the set input 7224 of register 7226. The square wave signal comes into this portion of the circuit at input 7228. At the high voltage of the square wave, capacitor 7230 will charge. At the low portion of the voltage signal, the amount that capacitor 7230 discharges depends on the time constant determined by capacitor 7230 and resistor 7232. If the frequency is lower than the cutoff, f1, the voltage 7234 held by capacitor 7230 will already be discharged by the time the signal voltage goes high again, and the input to flip-flop 7236 at node 7234 will be zero. In that case, the output 7238 will always be zero. If the frequency of input signal 7228 is above f1, capacitor 7230 is still holding the high voltage at node 7234 when the next high voltage occurs, causing output 7238 to become a logic 1. Flip-flop 7240 has a similar topology at its inputs, with capacitor 7242 and resistor 7244. A different value capacitor and/or resistor can be chosen, so that the cutoff frequency, f2, for flip-flop 7240 to output a 1, is different from f1. For example, f2 may be higher than f1. Outputs 7238 and 7246 are fed into NOR gate 7248. When the frequency of the incoming signal is below f1, outputs 7238 and 7246 are both logic 0, and the NOR gate output 7224 will be a logic 0. This ensures that any signal with a frequency below f1 will not activate the wakeup signal.

At a frequency of between f1 and f2, output 7238 will be a 1, while output 7246 will be a 0, causing NOR output 7224 to become a logic 1. This goes to the set input of register 7226 and activates the wakeup command. Because a pulse is needed so that a sleep command can be subsequently sent to the reset input of register 7226 without problems, the NOR output must then be driven to a logic 0. In order to do this, the wakeup signal sent to circuit input 7228 goes up to a frequency above f2 after the period between f1 and f2. This causes outputs 7238 and 7246 to both become logic 1, causing NOR output 7224 to become a logic 0. The higher frequency can be the same as normal communication frequency, which is convenient since after wakeup, a command is typically sent.

The frequencies f1 and f2 are chosen so all signals which may be present on S1 and S2 except for the wakeup signal will not fall in the range from f1 to f2.

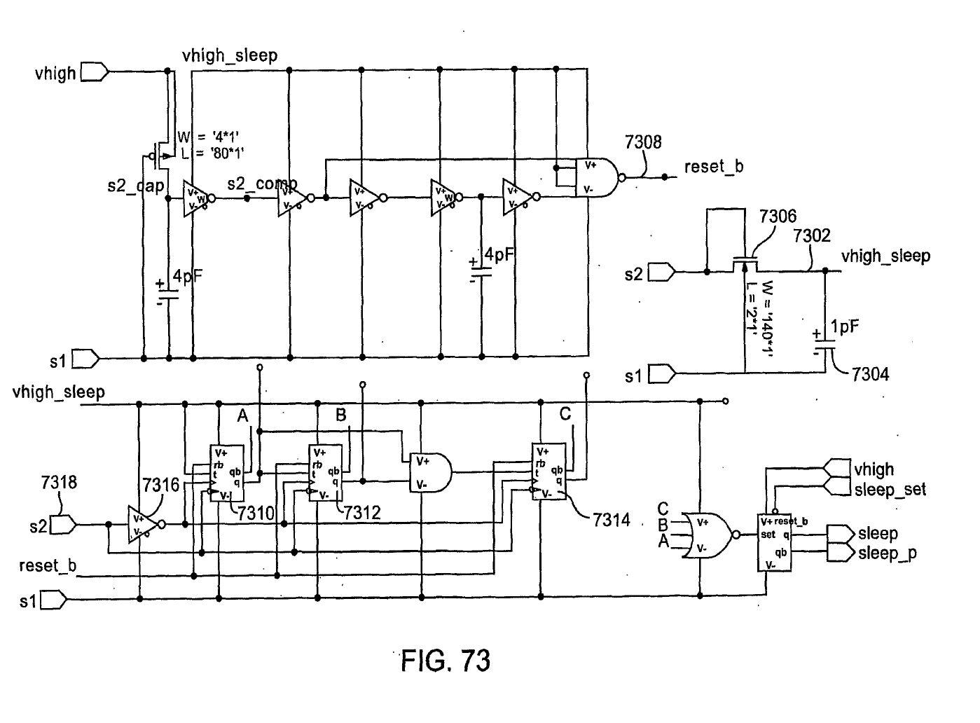

Another embodiment of the wakeup circuitry is shown in FIG. 73. When the device is powered up for the first time or after being in sleep mode, Vhigh_sleep 7302 goes up. There is a relatively small holding capacitor 7304 and a small diode 7306, so the voltage goes up quickly. When it does, there is a one-shot at reset_b 7308. The reset_b pulse clears registers 7310, 7312, and 7314. Registers 7310, 7312, and 7314 are arranged as a counter, counting from 0 to 7. When they count to 7, the wakeup command is issued. If there is only one pulse, or a pulse at a low frequency, such as a pacing pulse, vhigh_sleep 7302 drains very quickly. When that happens, flip-flops 7310, 7312, and 7314 go to zero as well. The next time a pulse goes through, the count starts over. However, if 7 pulses are sent through vhigh_sleep at a high enough frequency, the counter arrangement of flip-flops 7310, 7312, and 7314 will count to 7 and the wakeup command will be issued. Any frequency can be chosen as the cutoff above which the wakeup command will be issued. A frequency can be chosen which will be higher than the frequency of other pulses, such as pacing pulses, that may be on the line.

The input to the logic is inverter 7316. There can be an issue that if S2 7318 is between the rail voltages, it can cause a continual drain through inverter 7316. A signal must be sent along S2 that is temporarily higher than vhigh_sleep. By going from 0 to 5V, S2 will be at 5V, while vhigh_sleep will be at 4V, giving a clear logic 1. By always going 0 to 5V, it ensures that the signal remains a logic signal and will not cause current drain. In using a wakeup signal at about 5V, this circuit can be used at a lower voltage than the circuit in FIG. 72.

### Electrode Satellite Structures

Embodiments of the invention further include electrode assemblies, such as electrode satellite structures, where the structures include an integrated circuit control device, e.g., including a circuit as reviewed above, and at least one electrode element. As such, the satellite structures include control circuitry, e.g., in the form of an IC (e.g., an IC inside of the support), such that the satellite structure is addressable. In certain embodiments, the structure includes two or more electrode elements, such as three or more electrode elements, including four or more electrode elements, e.g., where the structure is a segmented electrode structure.

As reviewed above, the integrated circuit may be hermetically sealed or protected. Embodiments of hermetically sealed IC chips include, but are not limited to, those described in PCT application serial PCT/US2005/046815 titled “Implantable Hermetically Sealed Structures” and filed on Dec. 22, 2005, the description of hermetically sealed structures provided in this application being specifically incorporated herein by reference.

As summarized above, the invention provides implantable medical devices that include the electrode structures as described above. By implantable medical device is meant a device that is configured to be positioned on or in a living body, where in certain embodiments the implantable medical device is configured to be implanted in a living body. Embodiments of the implantable devices are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable devices are configured to maintain functionality when implanted at a physiological site for a period ranging from about 1 to about 80 years or longer, such as from about 5 to about 70 years or longer, and including for a period ranging from about 10 to about 50 years or longer. The dimensions of the implantable medical devices of the invention may vary. However, because the implantable medical devices are implantable, the dimensions of certain embodiments of the devices are not so big such that the device cannot be positioned in an adult human.

### Vascular Leads

Embodiments of the invention also include medical carriers that include one or more electrode satellite structures, e.g., as described above. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, etc.

In certain embodiments of the subject systems, one or more sets of electrode satellites as described above are electrically coupled to at least one elongated conductive member, e.g., an elongated conductive member present in a lead, such as a cardiovascular lead. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

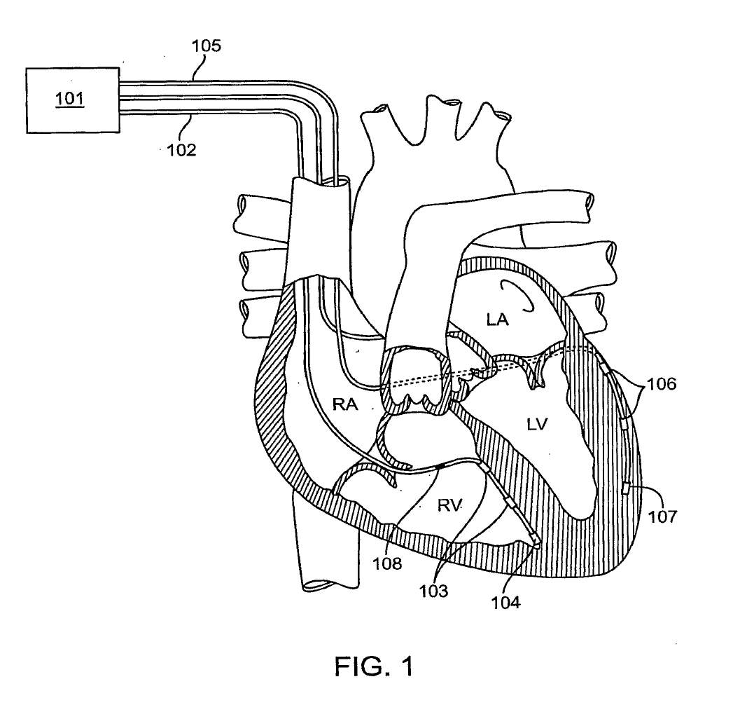

FIG. 2 illustrates an external view of a number of exemplary pacing satellites, in accordance with a multiplex lead embodiment of the present invention. According to one embodiment, a pacing lead 200 (e.g., right ventricular lead 102 or left ventricular lead 105 of FIG. 1) accommodates two bus wires S1 and S2, which are coupled to a number (e.g., eight) of satellites, such as satellite 202. FIG. 2 also shows satellite 202 with an enlarged view. Satellite 202 includes electrodes 212, 214, 216, and 218, located in the four quadrants of the cylindrical outer walls of satellite 202 and supported by a support structure of the invention. Each satellite also contains a control chip inside the structure which communicates with a pacing and signal-detection system to receive configuration signals that determine which of the four electrodes are to be coupled to bus wires S1 or S2.

The configuration signals, the subsequent pacing pulse signals, and the analog signals collected by the electrodes can all be communicated through bus wires S1 and S2, in either direction. Although shown in a symmetrical arrangement, electrodes 212, 214, 216 and 218 may be offset along lead 200 to minimize capacitive coupling among these electrodes. The quadrant arrangement of electrodes allows administering pacing current via electrodes oriented at a preferred direction, for example, away from nerves, or facing an electrode configured to sink the pacing current. Such precise pacing allows low-power pacing and minimal tissue damage caused by the pacing signal.

The leads may further include a variety of different effector elements, which elements may employ the satellites or structures distinct from the satellites. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

### Implantable Pulse Generators

Embodiments of the invention further include implantable pulse generators. Implantable pulse generators may include: a housing which includes a power source and an electrical stimulus control element; one or more vascular leads as described above, e.g., 2 or more vascular leads, where each lead is coupled to the control element in the housing via a suitable connector, e.g., an IS-1 connector. In certain embodiments, the implantable pulse generators are ones that are employed for cardiovascular applications, e.g., pacing applications, cardiac resynchronization therapy applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

An implantable pulse generator according to an embodiment of the invention is depicted in FIG. 1. FIG. 1 illustrates the locations of a number of pacing satellites incorporated in multi-electrode pacing leads, in accordance with an embodiment of the present invention. A pacing and signal detection system 101 provides extra-cardiac communication and control elements for the overall system. In some embodiments, pacing and signal detection system 101 may be, for example, a pacing can of a pacemaker residing in an external or extra-corporeal location.

Right ventricular lead 102 emerges from pacing and signal detection system 101 and travels from a subcutaneous location from pacing and signal detection system 101 into the patient's body (e.g., preferably, a subclavian venous access), and through the superior vena cava into the right atrium. From the right atrium, right ventricle lead 102 is threaded through the tricuspid valve to a location along the walls of the right ventricle. The distal portion of right ventricular lead 102 is preferably located along the intra-ventricular septum, terminating with a fixation in the right ventricular apex. Right ventricular lead 102 includes satellites positioned at locations 103 and 104. The number of satellites in ventricular lead 102 is not limited, and may be more or less than the number of satellites shown in FIG. 1.

Similarly, left ventricular lead 105 emerges from pacing and signal detection system 101, following substantially the same route as right ventricular lead 102 (e.g., through the subclavian venous access and the superior vena cava into the right atrium). In the right atrium, left ventricular lead 105 is threaded through the coronary sinus around the posterior wall of the heart in a cardiac vein draining into the coronary sinus. Left ventricular lead 105 is provided laterally along the walls of the left ventricle, which is likely to be an advantageous position for bi-ventricular pacing. FIG. 1 shows satellites positioned at locations 106 and 107 along left ventricular lead 105. Right ventricular lead 102 may optionally be provided with pressure sensor 108 in the right ventricle. A signal multiplexing arrangement allows a lead to include such active devices (e.g., pressure sensor 108) for pacing and signal collection purposes (e.g., right ventricular lead 102). Pacing and signal detection system 101 communicates with each of the satellites at locations 103, 104, 106 and 107. The electrodes controlled by the satellites may also be used to detect cardiac depolarization signals. Additionally, other types of sensors, such as an accelerometer, strain gauge, angle gauge, temperature sensor, can be included in any of the leads.

In the above system, the device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters; the disclosures of which are herein incorporated by reference), to the proximal end of electrode lead 105. The proximal end of electrode lead 105 connects to a pacemaker 101, e.g., via an IS-1 connector.

During certain embodiments of use, the electrode lead 105 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The electrode lead 105 is slid over the guidewire into the left ventricle cardiac vein and tested until an optimal location for CRT is found. Once implanted a multi-electrode lead 105 still allows for continuous readjustments of the optimal electrode location.

The electrode lead 102 is placed in the right ventricle of the heart. In this view, the electrode lead 102 is provided with one or multiple electrodes 103,104.

Electrode lead 102 is placed in the heart in a procedure similar to the typical placement procedures for cardiac right ventricle leads. Electrode lead 102 is placed in the heart using the standard cardiac lead devices which include introducers, guide catheters, guidewires, and/or stylets. Electrode lead 102 is inserted into the clavicle vein, through the superior vena cava, through the right atrium and down into the right ventricle. Electrode lead 102 is positioned under fluoroscopy into the location the clinician has determined is clinically optimal and logistically practical for fixating the electrode lead 102.

Summarizing aspects of the above description, in using the implantable pulse generators of the invention, such methods include implanting an implantable pulse generator e.g., as described above, into a subject; and the implanted pulse generator, e.g., to pace the heart of the subject, to perform cardiac resynchronization therapy in the subject, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various methods of operation of these applications being herein incorporated by reference and applicable for use of the present devices.

### Systems

Also provided are systems that include one more devices as described above, such as an implantable pulse generator. The systems of the invention may be viewed as systems for communicating information within the body of subject, e.g., human, where the systems include both a first implantable medical device, such as an IPG device described above, that includes a transceiver configured to transmit and/or receive a signal; and a second device comprising a transceiver configured to transmit and/or receive a signal. The second device may be a device that is inside the body, on a surface of the body or separate from the body during use.

Also provided are methods of using the systems of the invention. The methods of the invention generally include: providing a system of the invention, e.g., as described above, that includes first and second medical devices, one of which may be implantable; and transmitting a signal between the first and second devices. In certain embodiments, the transmitting step includes sending a signal from the first to said second device. In certain embodiments, the transmitting step includes sending a signal from the second device to said first device. The signal may transmitted in any convenient frequency, where in certain embodiments the frequency ranges from about 400 to about 405 MHz. The nature of the signal may vary greatly, and may include one or more data obtained from the patient, data obtained from the implanted device on device function, control information for the implanted device, power, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

### Methods of Making

The subject circuits, structures and devices described herein may be fabricated using any convenient protocol.

Aspects of the invention include methods of making a vascular lead electrode satellite, where the method includes providing an electrode support as described above and positioning an electrode element in a recess of the support, and in certain embodiments additionally includes placing an IC (such as the integrated circuits reviewed above) in the support such that the IC is electrically coupled to the electrode element(s) in the recess(es) of the support. In certain embodiments, the positioning step includes fitting a premade electrode element into the recess, e.g., by sliding the electrode into the recess. As such, a premade electrode element, such as a petal electrode as described in PCT/US2005/46811 titled “Implantable Addressable Segmented Electrodes” filed on Dec. 22, 2005, may be slid into the recess to produce the desired electrode structure. In certain embodiments, the methods include producing electrodes in recesses of the support, e.g., via a deposition protocol, such as cathodic arc deposition. Further descriptions of methods of producing electrode assemblies are provided in provisional application Ser. No. 60/865,760 filed on Nov. 14, 2006, the disclosure of which is herein incorporated by reference.

### Kits

Also provided are kits that include the circuits and/or implantable medical devices and systems or components thereof, e.g., that include the subject circuits, e.g., as reviewed above. In certain embodiments, the kits further include at least a control unit, e.g., in the form of a pacemaker can.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An implantable integrated circuit, said integrated circuit comprising:
a power extraction functional block;
an energy storage functional block;
a communication functional block; and
a device configuration functional block;
wherein said functional blocks are all present in a single integrated circuit on an intraluminal-sized support.

2. The integrated circuit according to claim 1, wherein substantially all of the functions of power extraction, energy storage, communication and device configuration employed by said circuit during use are provided by said single integrated circuit.

3. The integrated circuit according to claim 1, further comprising integrated corrosion protection films.

4. (canceled)

5. The integrated circuit according to claim 1, wherein a device configuration provided by said integrated circuit is functional without power being applied to said integrated circuit.

6. The integrated circuit according to claim 1, wherein a default configuration connecting one supply terminal to one or more effector electrodes is set in said integrated circuit upon power up of said circuit.

7. The integrated circuit according to claim 1, wherein said communication functional block employs an alternating current at a frequency above about 15 kHz.

8. The integrated circuit according to claim 1, wherein said device configuration functional block is configured to control one or more effectors.

9. The integrated circuit according to claim 8, wherein said integrated circuit further comprises a functional block that enables stimulation of tissue via said one or more effectors.

10. The integrated circuit according to claim 8, wherein said integrated circuit further comprises a functional block that enables low voltage transmission from tissue to said integrate circuit.

11. The integrated circuit according to claim 9, wherein said integrated circuit provides substantially charge-balanced transmission of a stimulation pulse.

12. The integrated circuit according to claim 8, wherein said device configuration block comprises a switching block between supply terminals and one or more effectors.

13. The integrated circuit according to claim 12, wherein said switching block comprises switching elements each comprised of two transistors between each effector and supply terminal.

14. The integrated circuit according to claim 13, wherein said two transistors share a common bulk that is electrically isolated from all other circuits.

15. The integrated circuit according to claim 14, wherein said two transistors comprise gates that are electrically connected.

16. The integrated circuit according to claim 15, wherein said two transistors comprise sources that are connected.

17. The integrated circuit according to claim 14, wherein said common bulk is electrically connected to a common source terminal.

18. The integrated circuit according to claim 15, wherein a control voltage applied to said gates is referenced to a voltage on said supply terminal.

19. The integrated circuit according to claim 1, further comprising a sleep functional block.

20. The integrated circuit according to claim 19, further comprising a wakeup functional block.

21. (canceled)

22. The integrated circuit according to claim 1, further comprising a current limiting functional block.

23. The integrated circuit according to claim 1, further comprising a voltage-clamping functional block.

24. The integrated circuit according to claim 1, further comprising a fault recovery functional block.

25. The integrated circuit according to claim 24, wherein said fault recovery functional block is configured to electrically isolate failed circuits or wires.

26. The integrated circuit according to claim 1, wherein said intraluminal-sized support has a largest surface area ranging from about 0.05 to about 5 mm².

27. The integrated circuit according to claim 1, wherein said integrated circuit is configured to have an average power consumption of about 100 μW or less.

28. The integrated circuit according to claim 1, wherein said integrated circuit is configured to have an average current draw while maintaining its configuration state of about 1 nA or less.

29. The integrated circuit according to claim 1, wherein said integrated circuit is configured to have an average current draw when the configuration state of the device is being changed that ranges from about 1 μA to about 100 μA.

30. (canceled)

31. The integrated circuit according to claim 1, wherein said integrated circuit is configured to operate a multi-effector satellite.

32-39. (canceled)

40. An implantable effector unit comprising:
(a) an integrated circuit, said integrated circuit comprising:
(i) a power extraction functional block;
(ii) an energy storage functional block;
(iii) a communication functional block; and
(iv) a device configuration functional block;
wherein said functional blocks are all present in a single integrated circuit on an intraluminal-sized support; and
(b) at least one effector coupled to said integrated circuit.

41. The implantable effector unit according to claim 40, wherein said effector unit comprises two or more effectors coupled to said integrated circuit.

42. The implantable effector unit according to claim 41, wherein said two or more effectors are electrodes.

43. The implantable effector unit according to claim 42, wherein said two or more electrodes are segmented electrodes.

44-50. (canceled)

51. An elongated flexible structure comprising a proximal end and a distal end, and at least one electrode assembly comprising:
(a) an integrated circuit, said integrated circuit comprising:
(i) a power extraction functional block;
(ii) an energy storage functional block;
(iii) a communication functional block; and
(iv) a device configuration functional block;
wherein said functional blocks are all present in a single integrated circuit on an intraluminal-sized support; and
(b) at least one two electrodes coupled to said integrated circuit.

52. The elongated flexible structure according to claim 51, wherein said structure is a vascular lead.

53. The elongated flexible structure according to claim 52, wherein said vascular lead comprises 2 or more electrode assemblies.

54. The elongated flexible structure according to claim 53, wherein said vascular lead is a multiplex vascular lead.

55. The elongated flexible structure according to claim 54, wherein said multiplex lead has 3 or less wires.

56. The elongated flexible structure according to claim 55, wherein said vascular lead includes only 2 wires.

57. The elongated flexible structure according to claim 55, wherein said vascular lead includes only 1 wire.

58. The elongated flexible structure according to claim 51, wherein said vascular lead includes an IS-1 connector at said proximal end.

59-69. (canceled)

70. A kit comprising:
(a) a housing comprising a power source and an electrical stimulus control element; and
(b) a vascular lead comprising an elongated flexible structure comprising a proximal end and a distal end, and at least one electrode assembly comprising:
(i) an integrated circuit, said integrated circuit comprising a power extraction functional block, an energy storage functional block, a communication functional block, a device configuration functional block, wherein said functional blocks are all present in a single integrated circuit on an intraluminal-sized support; and
(ii) at least one two electrodes coupled to said integrated circuit.
